(19) United States
(12) Patent Application Publication
(10) Pub. No.: US 2022/0241413 A1
(43) Pub. Date: Aug. 4, 2022

(54) COMBINATION THERAPIES USING CD-38 ANTIBODIES

(71) Applicant: Takeda Pharmaceutical Company Limited, Osaka (JP)

(72) Inventors: Antonio Palumbo, Osaka (JP); Kristina Allikmets, Osaka (JP); Deborah Berg, Osaka (JP); Eric Fedyk, Osaka (JP)

(21) Appl. No.: 17/617,811

(22) PCT Filed: Jun. 9, 2020
(86) PCT No.: PCT/IB20/00443

§ 371 Date: Dec. 9, 2021

Related U.S. Application Data
(60) Provisional application No. 62/859,631, filed on Jun. 10, 2019.

(51) Int. Cl. A61K 39/395; A61K 31/454; A61K 31/573; A61K 31/69; A61K 31/135; A61K 31/47; A61K 31/167; A61P 35/00

(52) U.S. Cl. CPC A61K 39/3955; A61K 31/454; A61K 31/573; A61K 31/69; A61K 2039/585; A61K 31/47; A61K 31/167; A61P 35/00; A61K 31/135

(57) ABSTRACT

Methods of administering isolated anti-CD38 antibodies in combination with lenalidomide or pomobdomide, and dexamethasone and, optionally, bortezomib, for the treatment of multiple myeloma.

### CROSS REFERENCE TO RELATED APPLICATION

This application claims priority to U.S. Provisional Patent Application No. 62/859,631, filed on Jun. 10, 2019, which is expressly incorporated by reference in its entirety.

### SEQUENCE LISTING

The instant application contains a Sequence Listing which has been submitted electronically in ASCII format and is hereby incorporated by reference in its entirety. Said ASCII copy, created on Jun. 8, 2020, is named 101588-5012-WO ST25.txt and is 18 kilobytes in size.

### FIELD OF THE INVENTION

The invention describes methods for treating multiple myeloma with combination therapies comprising administering anti-CD38 antibodies or antigen-binding fragments thereof.

### BACKGROUND OF THE INVENTION

Multiple myeloma (MM) is a rare, mostly incurable malignant disease of plasma cells in the bone marrow with substantial morbidity and mortality due to highly complex and diverse cytogenetic and molecular abnormalities. It accounts for approximately 1% of all cancers and approximately 13% of all hematologic cancers. Myeloma is most frequently diagnosed among people aged 65 to 74 years, with the median age being 69 years. The 5-year survival rate of patients with MM is approximately 50%. The clinical features of MM result from bone marrow infiltration by the malignant plasma cell clone, high levels of circulating immunoglobulin and/or free light chains (FLCs), depressed immunity, and end-organ damage. MM is characterized namely by hypercalcemia (resulting from bone resorption), renal impairment (commonly due to hypercalcemia, tumor infiltration, or hyperuricemia), anemia (due to tumor infiltration into the bone marrow and suppression of hematopoiesis by cytokines), and lytic bone lesions (due to osteoclast activating factors produced by the malignant plasma cells). Symptoms vary, but include bone pain, fractures, weakness, malaise, bleeding, anemia, and infections which result from immunodeficiency.

Prognosis in MM depends upon both patient factors and tumor variables at the time of diagnosis. Patient-related factors include age, performance status, and renal function. Tumor variables include disease stage, cytogenetic abnormalities, and extramedullary disease, as well as light chain and IgA disease. The normal aging process is associated with age-related changes in organ function and metabolic changes that can contribute to poor tolerability of cancer treatment, which can lead to poorer outcomes in the elderly. Further, chronological and biological age may not correspond, thus the presence of frailty, comorbidities, psychosocial functioning, and other disabilities can complicate the management of MM and therapy endurance. Because of age and comorbidities, elderly patients with MM are usually ineligible for autologous transplantation, therefore the treatment plan for this patient population consists of standard chemotherapy agents only. Although stem cell transplant (SCT) is an important part of treatment for patients under the age of 65, it is just one of many available treatment options. Some patients prefer to delay the comorbidity of SCT, thus the need for this treatment as well as its timing must be tailored to the patient's situation.

Treatment of MM is focused on containing proliferation of the myeloma cells and alleviating disease symptoms. Although there has been improvement in the outcome for MM patients in the last decade with a better understanding of the biology, improvement in treatment strategies, and the introduction of agents such as proteasome inhibitors (e.g., bortezomib, ixazomib, and carfilzomib); immunomodulatory drugs (e.g., lenalidomide and pomalidomide); and monoclonal antibodies (e.g., daratumumab and elotuzumab) the course of the disease is highly unpredictable among patients and is characterized by periods of remission without symptoms of variable duration and frequent relapses with symptoms. Eventually, the periods without disease symptoms shorten, and the illness becomes refractory to available therapies.

As MM progresses compounding factors such as lowered resistance to infection, anemia, and significant skeletal destruction add to the uniformly fatal prognosis. Additionally, although there have been improvements in overall survival (OS), there has been less benefit in patients 65 to 74 years and no benefit in patients 75 years and older as compared to patients less than 65 years of age. Therefore, MM remains a mostly incurable disease, underscoring the need and the urgency of developing new therapeutic options for these patients.

In patients with newly diagnosed multiple myeloma (NDMM) for whom SCT is not planned as an initial therapy, the standard treatment options include regimens containing 2 to 3 of the following agents (prescribing frequencies vary by country): bortezomib, lenalidomide, thalidomide, cyclophosphamide, and a corticosteroid (see, e.g., U.S. Pat. Nos. 10,232,041; 9,944,711; 9,289,490; 9,040,050; and 8,877,899; and U.S. Patent Publication Nos. 20180117150; 20190127479; 20180235986; 20180022823; 20170224817; 20170121417; 20170107295; 20170008966; 20160130362; 20160067205; 20150231235; 20140161819; 20130302318; 20130209355; 20100092489; and 20100028346; 20090148449). These regimens have been used to treat MM in the US and are well tolerated with antimyeloma activity. The IMiD-based regimen lenalidomide-dexamethasone (Len-Dex) is approved by the US Food and Drug Administration (FDA). The triplet combination, bortezomib (Velcade), lenalidomide, and dexamethasone, is used in the US based on improvement in progression-free survival (PFS). The choice of a specific therapy in patients with NDMM, including whether to give a doublet or triplet regimen, is often dictated by the aforementioned patient and tumor factors, including but not limited to age, comorbidities, frailty, drug availability, and prognosis based on an assessment of disease aggressiveness. Responses to therapies are transient, prompting a continued search for additional therapeutic options for patients, especially for elderly or newly diagnosed patients and those with comorbidity burdens.

CD38 is highly expressed on MM cells and expressed at a lower level in other hematopoietic cells, such as lymphoid and myeloid cells. This high level of expression on the myeloma cell surface supports CD38 as an appropriate therapeutic target, as validated by the US FDA approval in 2015 of the first anti-CD38 drug, daratumumab, as a monotherapy for patients with advanced relapsed and/or refractory multiple myeloma (RRMM). Subsequent marketing authorizations followed with daratumumab approval in combination with standard antimyeloma regimens for patients with less advanced RRMM as well as for those with NDMM who cannot receive a stem cell transplant. Recently, a safety analysis of full-dose, intravenous (IV) daratumumab added to the regimen bortezomib, lenalidomide, and dexamethasone (VRd), has demonstrated the combination to be tolerable in transplant eligible patients. Full doses of daratumumab as monotherapy or in combination have been shown to be safe with demonstrated activity in patients naïve to previous CD38-directed therapy. The most frequent adverse reactions (≥20%) reported with daratumumab, either as monotherapy or in combination with standard antimyeloma regimens, are infusion-related reactions (IRRs), neutropenia, thrombocytopenia, fatigue, nausea, diarrhea, constipation, vomiting, muscle spasms, arthralgia, back pain, pyrexia, chills, dizziness, insomnia, cough, dyspnea, peripheral edema, peripheral sensory neuropathy, and upper respiratory tract infections. Daratumumab can cause severe and serious IRRs, including anaphylactic reactions that have been reported in approximately half of all patients. In addition, daratumumab binds to CD38 on red blood cells (RBCs), a mechanism of action that yields persistently positive indirect Coombs test results for up to 6 months after the last daratumumab infusion. This binding may mask serum antigens, thus interfering with cross-matching and red blood cell antibody screening.

The monoclonal antibody AB79 binds with high affinity to CD38, exhibiting a different binding profile than daratumumab and unique pharmacodynamic characteristics. Preliminary evidence suggests that AB79 may be more selective and therefore more potent than daratumumab. In a phase 1 study in healthy subjects (Study AB79-101), AB79 reduced the levels of peripheral blood and natural killer (NK) cells >90% from baseline in all subjects receiving a single 0.06 mg/kg IV dose of AB79, with a maximum observed concentration (Cₘₐₓ) of 0.1 μg/mL. (U.S. Pat. No. 8,362,211; U.S. International Patent Application Serial Nos. PCT/US2019/013547 and PCT/US2019/024431). In contrast, comparable depletion of NK cells was not achieved with IV daratumumab administered to patients with RRMM at doses up to 24 mg/kg (mean Cₘₐₓ of >500 μg/mL). In healthy subjects, AB79 delivered SC also reduced levels of circulating plasmablasts in peripheral blood in a dose-dependent manner. Neutrophil, lymphocyte, monocyte, RBC, and platelet counts remained within normal ranges for all dose cohorts. Potent reduction of plasmablasts was observed across cohorts of patients in the RRMM trial (Study AB79-1501). Results indicated that peripheral blood plasmablasts were reduced 60% to 95% from baseline with AB79 SC at doses of 45 mg to 600 mg (approximately equivalent to 0.6 mg to 8 mg/kg). Consequently, AB79 may be more efficient at eliminating cells expressing high levels of CD38, which could manifest as higher activity on tumor cells (e.g., improved and deeper response rates across a population of patients with myeloma).

An additional benefit of higher potency of AB79 SC would be convenience of administration, as the desired clinical response could be obtained with less drug. To date, other anti-CD38 antibodies (e.g., daratumumab and isatuximab) must be administered as IV injections. The approved route of administration for daratumumab is an IV infusion given over several hours, which is not convenient for patients. The initial IV infusions of daratumumab can take 7 to 9 hours (including time for premedications), and subsequent doses require 4 to 6 hours per dose, with more time required if there are infusion reactions. To address this, clinical trials are investigating a formulation of daratumumab with human hyaluronidase to be administered subcutaneously (SC). The SC formulation of daratumumab that is currently in clinical development consists of 1800 mg of daratumumab in 15 mL of recombinant human hyaluronidase enzyme, which is required for creating a subcutaneous cavity to accommodate this relatively large volume; approximately 3 to 5 minutes in the clinic are required to administer this formulation via a syringe. The overall incidence of IRR with daratumumab SC is lower than with IV administration (reported incidence of 16% all grades, with 8% Grade 3 or higher, compared to an incidence upwards of 50% all grades with Grade 3 or higher as high as 9%). Injection site reactions, consisting of induration, erythema, discoloration, and hematomas were also reported in 16.7% of patients. Grade 3 and 4 treatment-emergent adverse events (TEAEs) included lymphopenia (20%) and thrombocytopenia, neutropenia, and hypertension (in 8% each). Notably, not all patients respond to daratumumab-based therapy, and many patients eventually develop progressive disease characterized by aggressive and highly symptomatic clinical features.

By contrast, IRRs have not been observed with AB79 SC dosed up to 600 mg. AB79 can be administered as one SC injection of approximately 2 mL without the need for hyaluronidase, for doses of less than or equal to 300 mg, lasting less than a minute in duration. Therefore, improved selectivity with AB79 may lead to improved efficacy and tolerability with more patient convenience as compared to that reported with daratumumab IV or SC.

Although early studies of AB79 in the treatment of myeloma look promising, given the shortfalls of other therapies and the fatal prognosis of MM, there remains a need for new agents or combinations thereof, including a new generation of CD-38 targeted therapies, with more selectivity, greater potency, less toxicity, and improved patient convenience to continue to improve clinical outcomes for all patients.

### SUMMARY OF THE INVENTION

Multiple-drug combinations are important in the frontline treatment of MM, demonstrating high response rates as well as prolonged PFS and OS. Enhancing available regimens with new drugs that provide synergistic nonoverlapping mechanisms of action (MOAs) may improve clinical benefit by improving response rates, which then may improve PFS and OS. A new generation of CD38-directed agents is required to delay progression of the disease, relieve symptoms, and to enhance the quality of life (QOL) of patients afflicted with this devastating, relentless disease.

Provided herein are methods of treating a subject having a CD38-positive hematological cancer, wherein the method comprises administering anti-CD38 antibodies or antigen-binding fragments thereof with combination therapies.

In one aspect, the present invention provides a method of treating subject having a CD38-positive hematological cancer, the method comprising administering to the subject a therapeutically effective amount of a) an anti-CD38 antibody, b) lenalidomide, and c) a corticosteroid for a time sufficient to treat the CD38-positive hematological cancer, wherein the anti-CD38 antibody comprises a variable heavy (VH) chain region comprising a CDR1 having the amino acid sequence of SEQ ID NO:3, a CDR2 having the amino acid sequence of SEQ ID NO:4, and a CDR3 having the amino acid sequence of SEQ ID NO:5; and a variable light (VL) chain region comprising a CDR1 having the amino acid sequence of SEQ ID NO:6, a CDR2 having the amino acid sequence of SEQ ID NO:7 and a CDR3 having the amino acid sequence of SEQ ID NO:8.

In an additional aspect, the present invention provides a method of treating a subject having a CD38-positive hematological cancer, the method comprising administering to the subject a therapeutically effective amount of a) an anti-CD38 antibody, b) pomalidomide, and c) a corticosteroid for a time sufficient to treat the CD38-positive hematological cancer, wherein the anti-CD38 antibody comprises a variable heavy (VH) chain region comprising a CDR1 having the amino acid sequence of SEQ ID NO:3, a CDR2 having the amino acid sequence of SEQ ID NO:4, and a CDR3 having the amino acid sequence of SEQ ID NO:5; and a variable light (VL) chain region comprising a CDR1 having the amino acid sequence of SEQ ID NO:6, a CDR2 having the amino acid sequence of SEQ ID NO:7 and a CDR3 having the amino acid sequence of SEQ ID NO:8.

In a further aspect, the VH chain region as described herein has the amino acid sequence of SEQ ID NO:9 and the VL chain region as described herein has the amino acid sequence of SEQ ID NO:10.

In additional aspect, the anti-CD38 antibody or antigen binding fragment thereof as described herein comprises a heavy chain amino acid sequence of SEQ ID NO:11 and a light chain amino acid sequence of SEQ ID NO:12.

In a further aspect, the anti-CD38 antibody as described herein is an IgG1, IgG2, IgG3 or IgG4 isotype.

In additional aspect, the anti-CD38 antibody as described herein is the IgG1 isotype.

In a further aspect, the anti-CD38 antibody or antigen binding fragment thereof as described herein is fully human.

In additional aspect, the anti-CD38 antibody is AB79.

In a further aspect, the CD38-positive hematological cancer as described herein is multiple myeloma.

In additional aspect, the CD38-positive hematological cancer as described herein is newly diagnosed multiple myeloma (NDMM) or naïve multiple myeloma.

In a further aspect, the CD38-positive hematological cancer as described herein has not been previously treated with hematological cancer drug.

In additional aspect, the CD38-positive hematological cancer as described herein has not been previously treated with a multiple myeloma drug.

In a further aspect, the subject as described herein has refractory or relapsed multiple myeloma (RRMM).

In additional aspect, the anti-CD38 antibody or antigen binding fragment thereof as described herein is administered at a dose of about 300 mg once weekly for two treatment cycles, at a dose of about 300 mg once every two weeks for subsequent four treatment cycles and at a dose of about 300 mg once every four weeks for any treatment cycles thereafter, wherein a treatment cycle is 28 days.

In a further aspect, the anti-CD38 antibody or antigen binding fragment thereof as described herein is administered subcutaneously.

In additional aspect, the anti-CD38 antibody or antigen binding fragment thereof as described herein is administered in the absence of a hyaluronidase.

In a further aspect, the lenalidomide as described herein is administered at a dose of about 2.5 to about 25 mg daily for 21 days of each treatment cycle for up to 8 treatment cycles, wherein the treatment cycle is 28 days.

In additional aspect, the lenalidomide as described herein is administered orally.

In a further aspect, the pomalidomide as described herein is administered daily in a therapeutically effective amount for 21 days of each treatment cycle for up to 8 treatment cycles, wherein the treatment cycle is 28 days.

In additional aspect, the pomalidomide as described herein is administered orally.

In a further aspect, the corticosteroid as described herein is dexamethasone.

In additional aspect, dexamethasone as described herein is administered at a dose of about 20-40 mg weekly for 1-8 treatment cycles, wherein the treatment cycle is 28 days.

In a further aspect, dexamethasone as described herein is administered at a dose of about 40 mg weekly for 1-8 treatment cycles, wherein the treatment cycle is 28 days.

In additional aspect, the dexamethasone as described herein is administered orally or intravenously. In one embodiment, dexamethasone is administered on days 1, 8, 15 and 22 of each treatment cycle.

In a further aspect, the method of treating subject having a CD38-positive hematological cancer as described herein further comprises administering a therapeutically effective amount of bortezomib.

In additional aspect, bortezomib as described herein is administered at a dose of about 0.7 to 1.3 mg/m² weekly for 3 weeks of 1-8 treatment cycles, wherein the treatment cycle is 28 days.

In a further aspect, the bortezomib as described herein is administered subcutaneously.

In additional aspect, the present invention provides the method of treating subject having a CD38-positive hematological cancer as described herein, wherein a) the anti-CD38 antibody or antigen binding fragment thereof is administered on days 1, 8, 15 and 22 of the first two treatment cycles, on days 1 and 15 of the subsequent four treatment cycles and on day 1 of any additional treatment cycles; b) lenolidomide is administered on days 1 to 21 of each treatment cycle; and c) the corticosteroid is administered on days 1, 8, 15 and 22 of each of 1-8 treatment cycles, wherein the treatment cycle is 28 days.

In a further aspect, the present invention provides the method of treating subject having a CD38-positive hematological cancer as described herein, wherein a) the anti-CD38 antibody or antigen binding fragment thereof is administered on days 1, 8, 15 and 22 of the first two treatment cycles, on days 1 and 15 of the subsequent four treatment cycles and on day 1 of any additional treatment cycles; b) pomolidomide is administered on days 1 to 21 of each treatment cycle; and c) the corticosteroid is administered on days 1, 8, 15 and 22 of each of 1-8 treatment cycles, wherein the treatment cycle is 28 days.

In additional aspect, the present invention provides the method of treating subject having a CD38-positive hematological cancer as described herein, wherein a) the anti-CD38 antibody or antigen binding fragment thereof is administered on days 1, 8, 15 and 22 of the first two treatment cycles, on days 1 and 15 of the subsequent four treatment cycles and on day 1 of any additional treatment cycles; b) lenolidomide is administered on days 1 to 21 of each treatment cycle; and c) the corticosteroid is administered on days 1, 8, 15 and 22 of each of 1-8 treatment cycles, wherein the treatment cycle is 28 days; and wherein the method further comprises administering a therapeutically effective amount of bortezomib.

In a further aspect, bortezomib as described herein is administered at a dose of about 0.7 to 1.3 mg/m² weekly for 3 weeks of 1-8 treatment cycles, wherein the treatment cycle is 28 days.

In additional aspect, bortezomib as described herein is administered on days 1, 8, and 15 of each treatment cycle.

In a further aspect, the CD38-positive hematological cancer as described herein is newly diagnosed multiple myeloma (NDMM), and wherein the subject is a patient for whom stem cell transplantation is not planned as initial therapy.

In additional aspect, the subject as described herein receives premedications 1 to 3 hours prior to the start of AB79 administration on each dosing day, and wherein the premedications comprise antipyretics and antihistamine. In some embodiments, the antipyretics is selected from the group consisting of acetaminophen, aspirin, ibuprofen, and naproxen.

In a further aspect, the antipyretics as described herein is acetaminophen, and is administered at a dose of about 650 to 1000 mg orally. In some embodiments, acetaminophen is administered at a dose of about 650 mg. In some embodiments, acetaminophen is administered at a dose of about 700 mg. In some embodiments, acetaminophen is administered at a dose of about 750 mg. In some embodiments, acetaminophen is administered at a dose of about 800 mg. In some embodiments, acetaminophen is administered at a dose of about 850 mg. In some embodiments, acetaminophen is administered at a dose of about 900 mg. In some embodiments, acetaminophen is administered at a dose of about 950 mg. In some embodiments, acetaminophen is administered at a dose of about 1000 mg.

In additional aspect, the antihistamine as described herein is diphenhydramine or equivalent, and is administered at a dose of about 25 mg to 50 mg orally or intravenously. In some embodiments, antihistamine is selected from the group consisting of brompheniramine, chlorpheniramine (Chlor-Trimeton), and diphenhydramine. In some embodiments, antihistamine is administered at a dose of about 25 mg. In some embodiments, antihistamine is administered at a dose of about 30 mg. In some embodiments, antihistamine is administered at a dose of about 35 mg. In some embodiments, antihistamine is administered at a dose of about 40 mg. In some embodiments, antihistamine is administered at a dose of about 45 mg. In some embodiments, antihistamine is administered at a dose of about 50 mg.

In a further aspect, the premedications as described herein further comprise montelukast or equivalent leukotriene inhibitor.

In an additional aspect, the montelukast or equivalent leukotriene inhibitor as described herein is administered at a dose of about 5 mg-15 mg. In some embodiments, montelukast or equivalent leukotriene inhibitor is administered at a dose of 5 mg. In some embodiments, montelukast or equivalent leukotriene inhibitor is administered at a dose of 10 mg. In some embodiments, montelukast or equivalent leukotriene inhibitor is administered at a dose of 15 mg.

In one aspect, the invention provides a method for treating MM, the method comprising the step of administering to a subject having MM a therapeutically effective amount of AB79 in combination with (a) lenalidomide and dexamethasone or (b) lenalidomide, dexamethasone, and bortezomib.

In one aspect, the invention provides a method for treating MM, the method comprising the step of administering to a subject having MM a therapeutically effective amount of AB79 in combination with pomalidomide and dexamethasone.

In one aspect, the invention provides a method for treating MM, the method comprising the step of administering to a subject having MM a therapeutically effective amount of AB79 subcutaneously, in combination with (a) lenalidomide and dexamethasone or (b) lenalidomide, dexamethasone, and bortezomib.

In one aspect, the invention provides a method for treating MM, the method comprising the step of administering to a subject having MM a therapeutically effective amount of AB79 subcutaneously, in combination with pomalidomide and dexamethasone.

In one aspect, the anti-CD38 antibody is administered in the absence of a hyaluronidase.

In an embodiment, the CD38-positive hematological cancer is multiple myeloma (MM). In an embodiment, the CD38-positive hematological cancer is newly diagnosed multiple myeloma (NDMM) or naïve multiple myeloma. In one embodiment, the CD38-positive hematological cancer is NDMM and stem cell transplantation is not planned as initial therapy for the patient having the CD38-positive hematological cancer. In an embodiment, the CD38-positive hematological cancer is refractory or relapsed multiple myeloma (RRMM). In an embodiment, the CD38-positive hematological cancer has not been previously treated with a hematological cancer drug. In an embodiment, the CD38-positive hematological cancer has not been previously treated with a multiple myeloma drug.

In an embodiment, the anti-CD38 antibody is administered at a dose of about 300 mg once weekly for first two treatment cycles, at a dose of about 300 mg once every two weeks for subsequent four treatment cycles and at a dose of about 300 mg once every four weeks for any treatment cycles thereafter, wherein the treatment cycle is 28 days. In an embodiment, the anti-CD38 antibody is administered subcutaneously. In an embodiment, the anti-CD38 antibody is AB79.

In an embodiment, lenalidomide is administered at a dose of about 2.5 to 25 mg daily for 21 days of each treatment cycle for up to 8 treatment cycles, wherein the treatment cycle is 28 days. In an embodiment, lenalidomide is administered at a dose of about 25 mg daily for 21 days of each treatment cycle for up to 8 treatment cycles, wherein the treatment cycle is 28 days. In an embodiment, the lenalidomide is administered orally.

In an embodiment, the dexamethasone is administered at a dose of about 20-40 mg weekly for 1-8 treatment cycles, wherein the treatment cycle is 28 days. In an embodiment, the dexamethasone is administered at a dose of about 20-40 mg weekly for 1, 2, 3, 4, 5, 6, 7 or 8 treatment cycles, wherein the treatment cycle is 28 days. In an embodiment, the dexamethasone is administered at a dose of about 20-40 mg weekly for 1 treatment cycle, wherein the treatment cycle is 28 days. In an embodiment, the dexamethasone is administered at a dose of about 20-40 mg weekly for 2 treatment cycles, wherein the treatment cycle is 28 days. In an embodiment, the dexamethasone is administered at a dose of about 20-40 mg weekly for 3 treatment cycles, wherein the treatment cycle is 28 days. In an embodiment, the dexamethasone is administered at a dose of about 20-40 mg weekly for 4 treatment cycles, wherein the treatment cycle is 28 days. In an embodiment, the dexamethasone is administered at a dose of about 20-40 mg weekly for 5 treatment cycles, wherein the treatment cycle is 28 days. In an embodiment, the dexamethasone is administered at a dose of about 20-40 mg weekly for 6 treatment cycles, wherein the treatment cycle is 28 days. In an embodiment, the dexamethasone is administered at a dose of about 20-40 mg weekly for 7 treatment cycles, wherein the treatment cycle is 28 days. In an embodiment, the dexamethasone is administered at a dose of about 20-40 mg weekly for 8 treatment cycles, wherein the treatment cycle is 28 days. In an embodiment, the dexamethasone is administered orally or intravenously. In an embodiment, dexamethasone is administered at a dose of about 20 mg weekly for 1, 2, 3, 4, 5, 6, 7 or 8 treatment cycles, wherein the treatment cycle is 28 days. In an embodiment, dexamethasone is administered at a dose of about 25 mg weekly for 1, 2, 3, 4, 5, 6, 7 or 8 treatment cycles, wherein the treatment cycle is 28 days. In an embodiment, dexamethasone is administered at a dose of about 30 mg weekly for 1, 2, 3, 4, 5, 6, 7 or 8 treatment cycles, wherein the treatment cycle is 28 days. In an embodiment, dexamethasone is administered at a dose of about 35 mg weekly for 1, 2, 3, 4, 5, 6, 7 or 8 treatment cycles, wherein the treatment cycle is 28 days. In an embodiment, dexamethasone is administered at a dose of about 40 mg weekly for 1, 2, 3, 4, 5, 6, 7 or 8 treatment cycles, wherein the treatment cycle is 28 days. In an embodiment, dexamethasone is administered at a dose of about 20 mg weekly for 8 treatment cycles, wherein the treatment cycle is 28 days. In an embodiment, dexamethasone is administered at a dose of about 25 mg weekly for 8 treatment cycles, wherein the treatment cycle is 28 days. In an embodiment, dexamethasone is administered at a dose of about 30 mg weekly for 8 treatment cycles, wherein the treatment cycle is 28 days. In an embodiment, dexamethasone is administered at a dose of about 35 mg weekly for 8 treatment cycles, wherein the treatment cycle is 28 days. In an embodiment, dexamethasone is administered at a dose of about 40 mg weekly for 8 treatment cycles, wherein the treatment cycle is 28 days. In an embodiment, the dexamethasone is administered orally or intravenously.

In an embodiment, the methods of the invention further comprise administering a therapeutically effective amount of bortezomib. In an embodiment, the bortezomib is Velcade® (Takeda). In an embodiment, the bortezomib is administered at a dose of about 0.7 to 1.3 mg/m² weekly for 3 weeks of 1-8 treatment cycles, wherein the treatment cycle is 28 days. In an embodiment, the bortezomib is administered at a dose of about 0.7 to 1.3 mg/m² weekly for 3 weeks of 1-8 treatment cycles, wherein the treatment cycle is 28 days. In an embodiment, the bortezomib is administered at a dose of about 0.7 mg/m² weekly for 3 weeks of 1, 2, 3, 4, 5, 6, 7, or 8 treatment cycles, wherein the treatment cycle is 28 days. In an embodiment, the bortezomib is administered at a dose of about 0.8 mg/m² weekly for 3 weeks of 1, 2, 3, 4, 5, 6, 7, or 8 treatment cycles, wherein the treatment cycle is 28 days. In an embodiment, the bortezomib is administered at a dose of about 0.9 mg/m² weekly for 3 weeks of 1, 2, 3, 4, 5, 6, 7, or 8 treatment cycles, wherein the treatment cycle is 28 days. In an embodiment, the bortezomib is administered at a dose of about 1.0 mg/m² weekly for 3 weeks of 1, 2, 3, 4, 5, 6, 7, or 8 treatment cycles, wherein the treatment cycle is 28 days. In an embodiment, the bortezomib is administered at a dose of about 1.1 mg/m² weekly for 3 weeks of 1, 2, 3, 4, 5, 6, 7, or 8 treatment cycles, wherein the treatment cycle is 28 days. In an embodiment, the bortezomib is administered at a dose of about 1.2 mg/m² weekly for 3 weeks of 1, 2, 3, 4, 5, 6, 7, or 8 treatment cycles, wherein the treatment cycle is 28 days. In an embodiment, the bortezomib is administered at a dose of about 1.3 mg/m² weekly for 3 weeks of 1, 2, 3, 4, 5, 6, 7, or 8 treatment cycles, wherein the treatment cycle is 28 days. In an embodiment, the bortezomib is administered subcutaneously.

In an embodiment, the bortezomib is administered at a dose of about 0.7-0.9 mg/m² weekly for 3 weeks of 1, 2, 3, 4, 5, 6, 7, or 8 treatment cycles, wherein the treatment cycle is 28 days. In an embodiment, the bortezomib is administered at a dose of about 0.7-1.0 mg/m² weekly for 3 weeks of 1, 2, 3, 4, 5, 6, 7, or 8 treatment cycles, wherein the treatment cycle is 28 days. In an embodiment, the bortezomib is administered at a dose of about 0.7-1.1 mg/m² weekly for 3 weeks of 1, 2, 3, 4, 5, 6, 7, or 8 treatment cycles, wherein the treatment cycle is 28 days. In an embodiment, the bortezomib is administered at a dose of about 0.7-1.2 mg/m² weekly for 3 weeks of 1, 2, 3, 4, 5, 6, 7, or 8 treatment cycles, wherein the treatment cycle is 28 days. In an embodiment, the bortezomib is administered at a dose of about 0.7-1.3 mg/m² weekly for 3 weeks of 1, 2, 3, 4, 5, 6, 7, or 8 treatment cycles, wherein the treatment cycle is 28 days. In an embodiment, the bortezomib is administered at a dose of about 0.8-1.0 mg/m² weekly for 3 weeks of 1, 2, 3, 4, 5, 6, 7, or 8 treatment cycles, wherein the treatment cycle is 28 days. In an embodiment, the bortezomib is administered at a dose of about 0.8-1.1 mg/m² weekly for 3 weeks of 1, 2, 3, 4, 5, 6, 7, or 8 treatment cycles, wherein the treatment cycle is 28 days. In an embodiment, the bortezomib is administered at a dose of about 0.8-1.2 mg/m² weekly for 3 weeks of 1, 2, 3, 4, 5, 6, 7, or 8 treatment cycles, wherein the treatment cycle is 28 days. In an embodiment, the bortezomib is administered at a dose of about 0.8-1.3 mg/m² weekly for 3 weeks of 1, 2, 3, 4, 5, 6, 7, or 8 treatment cycles, wherein the treatment cycle is 28 days. In an embodiment, the bortezomib is administered at a dose of about 0.9-1.1 mg/m² weekly for 3 weeks of 1, 2, 3, 4, 5, 6, 7, or 8 treatment cycles, wherein the treatment cycle is 28 days. In an embodiment, the bortezomib is administered at a dose of about 0.9-1.2 mg/m² weekly for 3 weeks of 1, 2, 3, 4, 5, 6, 7, or 8 treatment cycles, wherein the treatment cycle is 28 days. In an embodiment, the bortezomib is administered at a dose of about 0.9-1.3 mg/m² weekly for 3 weeks of 1, 2, 3, 4, 5, 6, 7, or 8 treatment cycles, wherein the treatment cycle is 28 days. In an embodiment, the bortezomib is administered at a dose of about 1.0-1.2 mg/m² weekly for 3 weeks of 1, 2, 3, 4, 5, 6, 7, or 8 treatment cycles, wherein the treatment cycle is 28 days. In an embodiment, the bortezomib is administered at a dose of about 1.0-1.3 mg/m² weekly for 3 weeks of 1, 2, 3, 4, 5, 6, 7, or 8 treatment cycles, wherein the treatment cycle is 28 days. In an embodiment, the bortezomib is administered at a dose of about 1.1-1.3 mg/m² weekly for 3 weeks of 1, 2, 3, 4, 5, 6, 7, or 8 treatment cycles, wherein the treatment cycle is 28 days. In some embodiments, the bortezomib is administered subcutaneously.

In an embodiment, a) the anti-CD38 antibody is administered on days 1, 8, 15 and 22 of the first two treatment cycles, on days 1 and 15 of the subsequent four treatment cycles and on day 1 of any additional treatment cycles; b) lenolidomide is administered on days 1 to 21 of each treatment cycle; and c) dexamethasone is administered on days 1, 8, 15 and 22 of each 1-8 treatment cycles, wherein the treatment cycle is 28 days. In one embodiment, dexamethasone is administered on days 1, 8, 15 and 22 of 1, 2, 3, 4, 5, 6, 7, or 8 treatment cycles, wherein the treatment cycle is 28 days. In one embodiment, dexamethasone is administered on days 1, 8, 15 and 22 of 1 treatment cycle, wherein the treatment cycle is 28 days. In one embodiment, dexamethasone is administered on days 1, 8, 15 and 22 of 2 treatment cycles, wherein the treatment cycle is 28 days. In one embodiment, dexamethasone is administered on days 1, 8, 15 and 22 of 3 treatment cycles, wherein the treatment cycle is 28 days. In one embodiment, dexamethasone is administered on days 1, 8, 15 and 22 of 4 treatment cycles, wherein the treatment cycle is 28 days. In one embodiment, dexamethasone is administered on days 1, 8, 15 and 22 of 5 treatment cycles, wherein the treatment cycle is 28 days. In one embodiment, dexamethasone is administered on days 1, 8, 15 and 22 of 6 treatment cycles, wherein the treatment cycle is 28 days. In one embodiment, dexamethasone is administered on days 1, 8, 15 and 22 of 7 treatment cycles, wherein the treatment cycle is 28 days. In one embodiment, dexamethasone is administered on days 1, 8, 15 and 22 of 8 treatment cycles, wherein the treatment cycle is 28 days.

In an embodiment, a) the anti-CD38 antibody is administered on days 1, 8, 15 and 22 of the first two treatment cycles, on days 1 and 15 of the subsequent four treatment cycles and on day 1 of any additional treatment cycles; b) lenolidomide is administered on days 1 to 21 of each treatment cycle; c) dexamethasone is administered on days 1, 8, 15 and 22 of each 1-8 treatment cycles; and d) bortezomib is administered on days 1, 8 and 15 of each 1-8 treatment cycles, wherein the treatment cycle is 28 days. In one embodiment, dexamethasone is administered on days 1, 8, 15 and 22 of 1, 2, 3, 4, 5, 6, 7, or 8 treatment cycles, wherein the treatment cycle is 28 days. In one embodiment, dexamethasone is administered on days 1, 8, 15 and 22 of 1 treatment cycle, wherein the treatment cycle is 28 days. In one embodiment, dexamethasone is administered on days 1, 8, 15 and 22 of each of 2 treatment cycles, wherein the treatment cycle is 28 days. In one embodiment, dexamethasone is administered on days 1, 8, 15 and 22 of each of 3 treatment cycles, wherein the treatment cycle is 28 days. In one embodiment, dexamethasone is administered on days 1, 8, 15 and 22 of each of 4 treatment cycles, wherein the treatment cycle is 28 days. In one embodiment, dexamethasone is administered on days 1, 8, 15 and 22 of each of 5 treatment cycles, wherein the treatment cycle is 28 days. In one embodiment, dexamethasone is administered on days 1, 8, 15 and 22 of each of 6 treatment cycles, wherein the treatment cycle is 28 days. In one embodiment, dexamethasone is administered on days 1, 8, 15 and 22 of each of 7 treatment cycles, wherein the treatment cycle is 28 days. In one embodiment, dexamethasone is administered on days 1, 8, 15 and 22 of each of 8 treatment cycles, wherein the treatment cycle is 28 days. In one embodiment, bortezomib is administered on days 1, 8, and 15 of 1, 2, 3, 4, 5, 6, 7, or 8 treatment cycles, wherein the treatment cycle is 28 days. In one embodiment, bortezomib is administered on days 1, 8, and 15 of 1 treatment cycle, wherein the treatment cycle is 28 days. In one embodiment, bortezomib is administered on days 1, 8, and 15 of each of 2 treatment cycles, wherein the treatment cycle is 28 days. In one embodiment, bortezomib is administered on days 1, 8, and 15 of each of 3 treatment cycles, wherein the treatment cycle is 28 days. In one embodiment, bortezomib is administered on days 1, 8, and 15 of each of 4 treatment cycles, wherein the treatment cycle is 28 days. In one embodiment, bortezomib is administered on days 1, 8, and 15 of each of 5 treatment cycles, wherein the treatment cycle is 28 days. In one embodiment, bortezomib is administered on days 1, 8, and 15 of each of 6 treatment cycles, wherein the treatment cycle is 28 days. In one embodiment, bortezomib is administered on days 1, 8, and 15 of each of 7 treatment cycles, wherein the treatment cycle is 28 days. In one embodiment, bortezomib is administered on days 1, 8, and 15 of each of 8 treatment cycles, wherein the treatment cycle is 28 days. In one embodiment, c) dexamethasone is administered on days 1, 8, 15 and 22 of 1 treatment cycle; and d) bortezomib is administered on days 1, 8 and 15 of 1 treatment cycles, wherein the treatment cycle is 28 days. In one embodiment, c) dexamethasone is administered on days 1, 8, 15 and 22 of 2 treatment cycles; and d) bortezomib is administered on days 1, 8 and 15 of 2 treatment cycles, wherein the treatment cycle is 28 days. In one embodiment, c) dexamethasone is administered on days 1, 8, 15 and 22 of 3 treatment cycles; and d) bortezomib is administered on days 1, 8 and 15 of 3 treatment cycles, wherein the treatment cycle is 28 days. In one embodiment, c) dexamethasone is administered on days 1, 8, 15 and 22 of 4 treatment cycles; and d) bortezomib is administered on days 1, 8 and 15 of 4 treatment cycles, wherein the treatment cycle is 28 days. In one embodiment, c) dexamethasone is administered on days 1, 8, 15 and 22 of 5 treatment cycles; and d) bortezomib is administered on days 1, 8 and 15 of 5 treatment cycles, wherein the treatment cycle is 28 days. In one embodiment, c) dexamethasone is administered on days 1, 8, 15 and 22 of 6 treatment cycles; and d) bortezomib is administered on days 1, 8 and 15 of 6 treatment cycles, wherein the treatment cycle is 28 days. In one embodiment, c) dexamethasone is administered on days 1, 8, 15 and 22 of 7 treatment cycles; and d) bortezomib is administered on days 1, 8 and 15 of 7 treatment cycles, wherein the treatment cycle is 28 days. In one embodiment, c) dexamethasone is administered on days 1, 8, 15 and 22 of 8 treatment cycles; and d) bortezomib is administered on days 1, 8 and 15 of 8 treatment cycles, wherein the treatment cycle is 28 days.

In an embodiment, the pomalidomide is administered daily in a therapeutically effective amount for 21 days of each treatment cycle for up to 8 treatment cycles, wherein the treatment cycle is 28 days. In an embodiment, the pomalidomide is administered orally.

In an embodiment, a) the anti-CD38 antibody is administered on days 1, 8, 15 and 22 of the first two treatment cycles, on days 1 and 15 of the subsequent four treatment cycles and on day 1 of any additional treatment cycles; b) pomolidomide is administered on days 1 to 21 of each treatment cycle; and c) the corticosteroid is administered on days 1, 8, 15 and 22 of each of 1-8 treatment cycles, wherein the treatment cycle is 28 days. In one embodiment, the corticosteroid is administered on days 1, 8, 15 and 22 of 1 treatment cycle, wherein the treatment cycle is 28 days. In one embodiment, the corticosteroid is administered on days 1, 8, 15 and 22 of each of 2 treatment cycles, wherein the treatment cycle is 28 days. In one embodiment, the corticosteroid is administered on days 1, 8, 15 and 22 of each of 3 treatment cycles, wherein the treatment cycle is 28 days. In one embodiment, the corticosteroid is administered on days 1, 8, 15 and 22 of each of 4 treatment cycles, wherein the treatment cycle is 28 days. In one embodiment, the corticosteroid is administered on days 1, 8, 15 and 22 of each of 5 treatment cycles, wherein the treatment cycle is 28 days. In one embodiment, the corticosteroid is administered on days 1, 8, 15 and 22 of each of 6 treatment cycles, wherein the treatment cycle is 28 days. In one embodiment, the corticosteroid is administered on days 1, 8, 15 and 22 of each of 7 treatment cycles, wherein the treatment cycle is 28 days. In one embodiment, the corticosteroid is administered on days 1, 8, 15 and 22 of each of 8 treatment cycles, wherein the treatment cycle is 28 days.

In one aspect, administering the anti-CD38 antibody treatment results in less than 60%, less than 50%, less than 40%, less than 30%, less than 25%, less than 20%, less than 15%, less than 10%, less than 5%, less than 4%, less than 3%, less than 2%, or less than 1% incidence of grade 3 or 4 of one or more treatment-related adverse events (TRAEs) or treatment-emergent adverse events (TEAEs) selected from the group consisting of anemia, hemolytic anemia, neutropenia, thrombocytopenia, fatigue, infusion-related reactions (IRRs), leukopenia, and lymphopenia. A TEAE is an adverse event that is observed or diagnosed up to about 30 days after the last dose of a drug regardless of cause. A TEAE may have any underlying cause related to the disease or treatment that is unrelated to the anti-CD38 antibody or it and can be specifically related to the anti-CD38 antibody. Suitably, administering the anti-CD38 antibody may result in less than 30% incidence of grade 3 or 4 of one or more treatment-emergent adverse events (TEAEs) selected from the group consisting of anemia, hemolytic anemia, thrombocytopenia, fatigue, infusion-related reactions (IRRs), leukopenia, and lymphopenia.

In one aspect, administering the anti-CD38 antibody treatment results in less than 10%, less than 9%, less than 8%, less than 7%, less than 6%, less than 5%, less than 4%, less than 3%, less than 2%, less than 1%, depletion of RBCs.

In one aspect, administering the anti-CD38 antibody treatment results in less than 10%, less than 9%, less than 8%, less than 7%, less than 6%, less than 5%, less than 4%, less than 3%, less than 2%, less than 1%, depletion of platelets.

In one aspect, the VH chain region of the anti-CD38 antibody or antigen binding fragment thereof comprises an amino acid sequence having at least 80% sequence identity to SEQ ID NO: 9 and the VL chain region of the anti-CD38 antibody or antigen binding fragment thereof comprises an amino acid sequence having at least 80% sequence identity to SEQ ID NO: 10. Suitably, the VH chain region of the anti-CD38 antibody or antigen binding fragment thereof may comprise an amino acid sequence having at least 85% sequence identity to SEQ ID NO: 9 and the VL chain region of the anti-CD38 antibody or antigen binding fragment thereof comprises an amino acid sequence having at least 85% sequence identity to SEQ ID NO: 10. Suitably, the VH chain region of the anti-CD38 antibody or antigen binding fragment thereof may comprise an amino acid sequence having at least 90% sequence identity to SEQ ID NO: 9 and the VL chain region of the anti-CD38 antibody or antigen binding fragment thereof comprises an amino acid sequence having at least 90% sequence identity to SEQ ID NO: 10. Suitably, the VH chain region of the anti-CD38 antibody or antigen binding fragment thereof may comprise an amino acid sequence having at least 95% sequence identity to SEQ ID NO: 9 and the VL chain region of the anti-CD38 antibody or antigen binding fragment thereof comprises an amino acid sequence having at least 95% sequence identity to SEQ ID NO: 10. Suitably, the VH chain region of the anti-CD38 antibody or antigen binding fragment thereof may comprise an amino acid sequence having at least 97% sequence identity to SEQ ID NO: 9 and the VL chain region of the anti-CD38 antibody or antigen binding fragment thereof comprises an amino acid sequence having at least 97% sequence identity to SEQ ID NO: 10. Suitably, the VH chain region of the anti-CD38 antibody or antigen binding fragment thereof may comprise an amino acid sequence having at least 99% sequence identity to SEQ ID NO: 9 and the VL chain region of the anti-CD38 antibody or antigen binding fragment thereof comprises an amino acid sequence having at least 99% sequence identity to SEQ ID NO: 10.

Suitably, the VH chain of the anti-CD38 antibody or antigen binding fragment thereof may comprise the CDR sequences as defined by SEQ ID NO: 3, SEQ ID NO: 4 and SEQ ID NO: 5 and the remainder of the sequence may have at least 80% sequence identity to SEQ ID NO: 9 and the VL chain of the anti-CD38 antibody or antigen binding fragment thereof may comprise the CDR sequences as defined by SEQ ID NO: 6, SEQ ID NO: 7 and SEQ ID NO: 8 and the remainder of the VL sequence may have at least 80% sequence identity to SEQ ID NO: 10. Suitably, the VH chain of the anti-CD38 antibody or antigen binding fragment thereof may comprise the CDR sequences as defined by SEQ ID NO: 3, SEQ ID NO: 4 and SEQ ID NO: 5 and the remainder of the sequence may have at least 85% sequence identity to SEQ ID NO: 9 and the VL chain of the anti-CD38 antibody or antigen binding fragment thereof may comprise the CDR sequences as defined by SEQ ID NO: 6, SEQ ID NO: 7 and SEQ ID NO: 8 and the remainder of the VL sequence may have at least 85% sequence identity to SEQ ID NO: 10. Suitably, the VH chain of the anti-CD38 antibody or antigen binding fragment thereof may comprise the CDR sequences as defined by SEQ ID NO: 3, SEQ ID NO: 4 and SEQ ID NO: 5 and the remainder of the sequence may have at least 90% sequence identity to SEQ ID NO: 9 and the VL chain of the anti-CD38 antibody or antigen binding fragment thereof may comprise the CDR sequences as defined by SEQ ID NO: 6, SEQ ID NO: 7 and SEQ ID NO: 8 and the remainder of the VL sequence may have at least 90% sequence identity to SEQ ID NO: 10. Suitably, the VH chain of the anti-CD38 antibody or antigen binding fragment thereof may comprise the CDR sequences as defined by SEQ ID NO: 3, SEQ ID NO: 4 and SEQ ID NO: 5 and the remainder of the sequence may have at least 95% sequence identity to SEQ ID NO: 9 and the VL chain of the anti-CD38 antibody or antigen binding fragment thereof may comprise the CDR sequences as defined by SEQ ID NO: 6, SEQ ID NO: 7 and SEQ ID NO: 8 and the remainder of the VL sequence may have at least 95% sequence identity to SEQ ID NO: 10. Suitably, the VH chain of the anti-CD38 antibody or antigen binding fragment thereof may comprise the CDR sequences as defined by SEQ ID NO: 3, SEQ ID NO: 4 and SEQ ID NO: 5 and the remainder of the sequence may have at least 97% sequence identity to SEQ ID NO: 9 and the VL chain of the anti-CD38 antibody or antigen binding fragment thereof may comprise the CDR sequences as defined by SEQ ID NO: 6, SEQ ID NO: 7 and SEQ ID NO: 8 and the remainder of the VL sequence may have at least 97% sequence identity to SEQ ID NO: 10. Suitably, the VH chain of the anti-CD38 antibody or antigen binding fragment thereof may comprise the CDR sequences as defined by SEQ ID NO: 3, SEQ ID NO: 4 and SEQ ID NO: 5 and the remainder of the sequence may have at least 99% sequence identity to SEQ ID NO: 9 and the VL chain of the anti-CD38 antibody or antigen binding fragment thereof may comprise the CDR sequences as defined by SEQ ID NO: 6, SEQ ID NO: 7 and SEQ ID NO: 8 and the remainder of the VL sequence may have at least 99% sequence identity to SEQ ID NO: 10.

In one aspect, the VH chain region of the anti-CD38 antibody or antigen binding fragment thereof has the amino acid sequence of SEQ ID NO: 9 or a variant thereof with up to three amino acid substitutions and the VL chain region of the anti-CD38 antibody or antigen binding fragment thereof has the amino acid sequence of SEQ ID NO:10 or a variant thereof with up to three amino acid substitutions.

In one aspect, the VH chain region of the anti-CD38 antibody or antigen binding fragment thereof has the amino acid sequence of SEQ ID NO:9 and the VL chain region of the anti-CD38 antibody or antigen binding fragment thereof has the amino acid sequence of SEQ ID NO:10.

In one aspect, the heavy chain of the anti-CD38 antibody or antigen binding fragment thereof comprises an amino acid sequence having at least 80% sequence identity to SEQ ID NO:11 and the light chain of the anti-CD38 antibody or antigen binding fragment thereof comprises an amino acid sequence having at least 80% sequence identity to SEQ ID NO:12. Suitably, the heavy chain of the anti-CD38 antibody or antigen binding fragment thereof comprises an amino acid sequence having at least 85% sequence identity to SEQ ID NO:11 and the light chain of the anti-CD38 antibody or antigen binding fragment thereof comprises an amino acid sequence having at least 85% sequence identity to SEQ ID NO:12. Suitably, the heavy chain of the anti-CD38 antibody or antigen binding fragment thereof comprises an amino acid sequence having at least 90% sequence identity to SEQ ID NO:11 and the light chain of the anti-CD38 antibody or antigen binding fragment thereof comprises an amino acid sequence having at least 90% sequence identity to SEQ ID NO:12. Suitably, the heavy chain of the anti-CD38 antibody or antigen binding fragment thereof comprises an amino acid sequence having at least 95% sequence identity to SEQ ID NO:11 and the light chain of the anti-CD38 antibody or antigen binding fragment thereof comprises an amino acid sequence having at least 95% sequence identity to SEQ ID NO:12. Suitably, the heavy chain of the anti-CD38 antibody or antigen binding fragment thereof comprises an amino acid sequence having at least 97% sequence identity to SEQ ID NO:11 and the light chain of the anti-CD38 antibody or antigen binding fragment thereof comprises an amino acid sequence having at least 97% sequence identity to SEQ ID NO:12. Suitably, the heavy chain of the anti-CD38 antibody or antigen binding fragment thereof comprises an amino acid sequence having at least 99% sequence identity to SEQ ID NO:11 and the light chain of the anti-CD38 antibody or antigen binding fragment thereof comprises an amino acid sequence having at least 99% sequence identity to SEQ ID NO:12.

Suitably, the heavy chain of the anti-CD38 antibody or antigen binding fragment thereof may comprise the CDR sequences as defined by SEQ ID NO: 3, SEQ ID NO: 4 and SEQ ID NO: 5 and the remainder of the heavy chain sequence may have at least 80% sequence identity to SEQ ID NO: 11 and the light chain of the anti-CD38 antibody or antigen binding fragment thereof may comprise the CDR sequences as defined by SEQ ID NO: 6, SEQ ID NO: 7 and SEQ ID NO: 8 and the remainder of the light chain sequence may have at least 80% sequence identity to SEQ ID NO: 12. Suitably, the heavy chain of the anti-CD38 antibody or antigen binding fragment thereof may comprise the CDR sequences as defined by SEQ ID NO: 3, SEQ ID NO: 4 and SEQ ID NO: 5 and the remainder of the heavy chain sequence may have at least 85% sequence identity to SEQ ID NO: 11 and the light chain of the anti-CD38 antibody or antigen binding fragment thereof may comprise the CDR sequences as defined by SEQ ID NO: 6, SEQ ID NO: 7 and SEQ ID NO: 8 and the remainder of the light chain sequence may have at least 85% sequence identity to SEQ ID NO: 12. Suitably, the heavy chain of the anti-CD38 antibody or antigen binding fragment thereof may comprise the CDR sequences as defined by SEQ ID NO: 3, SEQ ID NO: 4 and SEQ ID NO: 5 and the remainder of the heavy chain sequence may have at least 90% sequence identity to SEQ ID NO: 11 and the light chain of the anti-CD38 antibody or antigen binding fragment thereof may comprise the CDR sequences as defined by SEQ ID NO: 6, SEQ ID NO: 7 and SEQ ID NO: 8 and the remainder of the light chain sequence may have at least 90% sequence identity to SEQ ID NO: 12. Suitably, the heavy chain of the anti-CD38 antibody or antigen binding fragment thereof may comprise the CDR sequences as defined by SEQ ID NO: 3, SEQ ID NO: 4 and SEQ ID NO: 5 and the remainder of the heavy chain sequence may have at least 95% sequence identity to SEQ ID NO: 11 and the light chain of the anti-CD38 antibody or antigen binding fragment thereof may comprise the CDR sequences as defined by SEQ ID NO: 6, SEQ ID NO: 7 and SEQ ID NO: 8 and the remainder of the light chain sequence may have at least 95% sequence identity to SEQ ID NO: 12. Suitably, the heavy chain of the anti-CD38 antibody or antigen binding fragment thereof may comprise the CDR sequences as defined by SEQ ID NO: 3, SEQ ID NO: 4 and SEQ ID NO: 5 and the remainder of the heavy chain sequence may have at least 97% sequence identity to SEQ ID NO: 11 and the light chain of the anti-CD38 antibody may comprise the CDR sequences as defined by SEQ ID NO: 6, SEQ ID NO: 7 and SEQ ID NO: 8 and the remainder of the light chain sequence may have at least 97% sequence identity to SEQ ID NO: 12. Suitably, the heavy chain of the anti-CD38 antibody or antigen binding fragment thereof may comprise the CDR sequences as defined by SEQ ID NO: 3, SEQ ID NO: 4 and SEQ ID NO: 5 and the remainder of the heavy chain sequence may have at least 99% sequence identity to SEQ ID NO: 11 and the light chain of the anti-CD38 antibody or antigen binding fragment thereof may comprise the CDR sequences as defined by SEQ ID NO: 6, SEQ ID NO: 7 and SEQ ID NO: 8 and the remainder of the light chain sequence may have at least 99% sequence identity to SEQ ID NO: 12.

In one aspect, the anti-CD38 antibody or antigen binding fragment thereof comprises a heavy chain amino acid sequence of SEQ ID NO:11 or a variant thereof with up to three amino acid substitutions and a light chain amino acid sequence of SEQ ID NO:12 or a variant thereof with up to three amino acid substitutions.

In one aspect, the anti-CD38 antibody or antigen binding fragment thereof comprises a heavy chain amino acid sequence of SEQ ID NO:11 and a light chain amino acid sequence of SEQ ID NO:12.

In one aspect, the anti-CD38 antibody or antigen binding fragment thereof is fully human. In another aspect, the anti-CD38 antibody or antigen binding fragment thereof is humanized. In another aspect, the anti-CD38 antibody or antigen binding fragment thereof is affinity matured.

In one aspect, the anti-CD38 antibody or antigen binding fragment thereof does not cause hemolytic anemia or thrombocytopenia.

In one embodiment, the hematological cancer is multiple myeloma (MM). In one embodiment, the hematological cancer is newly diagnosed multiple myeloma (NDMM) or naïve multiple myeloma. In one embodiment, the hematological cancer is relapsed or refractory multiple myeloma (RRMM). In an embodiment, the method of the invention effectively treats one or more underlying symptoms of the MM, NDMM, or RRMM or other CD38-related disorder from which the patient is suffering. In one embodiment, the hematological cancer is NDMM and stem cell transplantation is not planned as initial therapy for the patient having the NDMM.

In one aspect, the therapeutically effective amount of anti-CD38 antibody or antigen binding fragment thereof is a dosage of about 300 milligrams (mgs). Suitably, the invention provides a unit dosage form of 300 mg.

In one aspect, a unit dosage form is provided in which the VH chain region of the anti-CD38 antibody or antigen binding fragment thereof comprises an amino acid sequence having at least 80% sequence identity to SEQ ID NO: 9 and the VL chain region of the anti-CD38 antibody or antigen binding fragment thereof comprises an amino acid sequence having at least 80% sequence identity to SEQ ID NO: 10. Suitably, a unit dosage form is provided in which the VH chain region of the anti-CD38 antibody or antigen binding fragment thereof may comprise an amino acid sequence having at least 85% sequence identity to SEQ ID NO: 9 and the VL chain region of the anti-CD38 antibody or antigen binding fragment thereof comprises an amino acid sequence having at least 85% sequence identity to SEQ ID NO: 10. Suitably, a unit dosage form is provided in which the VH chain region of the anti-CD38 antibody or antigen binding fragment thereof may comprise an amino acid sequence having at least 90% sequence identity to SEQ ID NO: 9 and the VL chain region of the anti-CD38 antibody or antigen binding fragment thereof comprises an amino acid sequence having at least 90% sequence identity to SEQ ID NO: 10. Suitably, a unit dosage form is provided in which the VH chain region of the anti-CD38 antibody or antigen binding fragment thereof may comprise an amino acid sequence having at least 95% sequence identity to SEQ ID NO: 9 and the VL chain region of the anti-CD38 antibody or antigen binding fragment thereof comprises an amino acid sequence having at least 95% sequence identity to SEQ ID NO: 10. Suitably, a unit dosage form is provided in which the VH chain region of the anti-CD38 antibody or antigen binding fragment thereof may comprise an amino acid sequence having at least 97% sequence identity to SEQ ID NO: 9 and the VL chain region of the anti-CD38 antibody or antigen binding fragment thereof comprises an amino acid sequence having at least 97% sequence identity to SEQ ID NO: 10. Suitably, a unit dosage form is provided in which the VH chain region of the anti-CD38 antibody or antigen binding fragment thereof may comprise an amino acid sequence having at least 99% sequence identity to SEQ ID NO: 9 and the VL chain region of the anti-CD38 antibody or antigen binding fragment thereof comprises an amino acid sequence having at least 99% sequence identity to SEQ ID NO: 10.

Suitably, a unit dosage form is provided in which the VH chain of the anti-CD38 antibody or antigen binding fragment thereof may comprise the CDR sequences as defined by SEQ ID NO: 3, SEQ ID NO: 4 and SEQ ID NO: 5 and the remainder of the sequence may have at least 80% sequence identity to SEQ ID NO: 9 and the VL chain of the anti-CD38 antibody or antigen binding fragment thereof may comprise the CDR sequences as defined by SEQ ID NO: 6, SEQ ID NO: 7 and SEQ ID NO: 8 and the remainder of the VL sequence may have at least 80% sequence identity to SEQ ID NO: 10. Suitably, a unit dosage form is provided in which the VH chain of the anti-CD38 antibody or antigen binding fragment thereof may comprise the CDR sequences as defined by SEQ ID NO: 3, SEQ ID NO: 4 and SEQ ID NO: 5 and the remainder of the sequence may have at least 85% sequence identity to SEQ ID NO: 9 and the VL chain of the anti-CD38 antibody or antigen binding fragment thereof may comprise the CDR sequences as defined by SEQ ID NO: 6, SEQ ID NO: 7 and SEQ ID NO: 8 and the remainder of the VL sequence may have at least 85% sequence identity to SEQ ID NO: 10. Suitably, a unit dosage form is provided in which the VH chain of the anti-CD38 antibody or antigen binding fragment thereof may comprise the CDR sequences as defined by SEQ ID NO: 3, SEQ ID NO: 4 and SEQ ID NO: 5 and the remainder of the sequence may have at least 90% sequence identity to SEQ ID NO: 9 and the VL chain of the anti-CD38 antibody or antigen binding fragment thereof may comprise the CDR sequences as defined by SEQ ID NO: 6, SEQ ID NO: 7 and SEQ ID NO: 8 and the remainder of the VL sequence may have at least 90% sequence identity to SEQ ID NO: 10. Suitably, a unit dosage form is provided in which the VH chain of the anti-CD38 antibody or antigen binding fragment thereof may comprise the CDR sequences as defined by SEQ ID NO: 3, SEQ ID NO: 4 and SEQ ID NO: 5 and the remainder of the sequence may have at least 95% sequence identity to SEQ ID NO: 9 and the VL chain of the anti-CD38 antibody or antigen binding fragment thereof may comprise the CDR sequences as defined by SEQ ID NO: 6, SEQ ID NO: 7 and SEQ ID NO: 8 and the remainder of the VL sequence may have at least 95% sequence identity to SEQ ID NO: 10. Suitably, a unit dosage form is provided in which the VH chain of the anti-CD38 antibody or antigen binding fragment thereof may comprise the CDR sequences as defined by SEQ ID NO: 3, SEQ ID NO: 4 and SEQ ID NO: 5 and the remainder of the sequence may have at least 97% sequence identity to SEQ ID NO: 9 and the VL chain of the anti-CD38 antibody may comprise the CDR sequences as defined by SEQ ID NO: 6, SEQ ID NO: 7 and SEQ ID NO: 8 and the remainder of the VL sequence may have at least 97% sequence identity to SEQ ID NO: 10. Suitably, a unit dosage form is provided in which the VH chain of the anti-CD38 antibody or antigen binding fragment thereof may comprise the CDR sequences as defined by SEQ ID NO: 3, SEQ ID NO: 4 and SEQ ID NO: 5 and the remainder of the sequence may have at least 99% sequence identity to SEQ ID NO: 9 and the VL chain of the anti-CD38 antibody or antigen binding fragment thereof may comprise the CDR sequences as defined by SEQ ID NO: 6, SEQ ID NO: 7 and SEQ ID NO: 8 and the remainder of the VL sequence may have at least 99% sequence identity to SEQ ID NO: 10.

In one aspect, a unit dosage form is provided in which the VH chain region of the anti-CD38 antibody or antigen binding fragment thereof has the amino acid sequence of SEQ ID NO: 9 or a variant thereof with up to three amino acid substitutions and the VL chain region of the anti-CD38 antibody or antigen binding fragment thereof has the amino acid sequence of SEQ ID NO:10 or a variant thereof with up to three amino acid substitutions.

In one aspect, a unit dosage form is provided in which the VH chain region of the anti-CD38 antibody or antigen binding fragment thereof has the amino acid sequence of SEQ ID NO:9 and the VL chain region of the anti-CD38 antibody or antigen binding fragment thereof has the amino acid sequence of SEQ ID NO:10.

In one aspect, a unit dosage form is provided in which the heavy chain of the anti-CD38 antibody or antigen binding fragment thereof comprises an amino acid sequence having at least 80% sequence identity to SEQ ID NO:11 and the light chain of the anti-CD38 antibody comprises an amino acid sequence having at least 80% sequence identity to SEQ ID NO:12. Suitably, a unit dosage form is provided in which the heavy chain of the anti-CD38 antibody or antigen binding fragment thereof comprises an amino acid sequence having at least 85% sequence identity to SEQ ID NO:11 and the light chain of the anti-CD38 antibody or antigen binding fragment thereof comprises an amino acid sequence having at least 85% sequence identity to SEQ ID NO:12. Suitably, a unit dosage form is provided in which the heavy chain of the anti-CD38 antibody or antigen binding fragment thereof comprises an amino acid sequence having at least 90% sequence identity to SEQ ID NO:11 and the light chain of the anti-CD38 antibody or antigen binding fragment thereof comprises an amino acid sequence having at least 90% sequence identity to SEQ ID NO:12. Suitably, a unit dosage form is provided in which the heavy chain of the anti-CD38 antibody or antigen binding fragment thereof comprises an amino acid sequence having at least 95% sequence identity to SEQ ID NO:11 and the light chain of the anti-CD38 antibody or antigen binding fragment thereof comprises an amino acid sequence having at least 95% sequence identity to SEQ ID NO:12. Suitably, a unit dosage form is provided in which the heavy chain of the anti-CD38 antibody or antigen binding fragment thereof comprises an amino acid sequence having at least 97% sequence identity to SEQ ID NO:11 and the light chain of the anti-CD38 antibody or antigen binding fragment thereof comprises an amino acid sequence having at least 97% sequence identity to SEQ ID NO:12. Suitably, a unit dosage form is provided in which the heavy chain of the anti-CD38 antibody or antigen binding fragment thereof comprises an amino acid sequence having at least 99% sequence identity to SEQ ID NO:11 and the light chain of the anti-CD38 antibody or antigen binding fragment thereof comprises an amino acid sequence having at least 99% sequence identity to SEQ ID NO:12.

Suitably, a unit dosage form is provided in which the heavy chain of the anti-CD38 antibody or antigen binding fragment thereof may comprise the CDR sequences as defined by SEQ ID NO: 3, SEQ ID NO: 4 and SEQ ID NO: 5 and the remainder of the heavy chain sequence may have at least 80% sequence identity to SEQ ID NO: 11 and the light chain of the anti-CD38 antibody or antigen binding fragment thereof may comprise the CDR sequences as defined by SEQ ID NO: 6, SEQ ID NO: 7 and SEQ ID NO: 8 and the remainder of the light chain sequence may have at least 80% sequence identity to SEQ ID NO: 12. Suitably, a unit dosage form is provided in which the heavy chain of the anti-CD38 antibody or antigen binding fragment thereof may comprise the CDR sequences as defined by SEQ ID NO: 3, SEQ ID NO: 4 and SEQ ID NO: 5 and the remainder of the heavy chain sequence may have at least 85% sequence identity to SEQ ID NO: 11 and the light chain of the anti-CD38 antibody or antigen binding fragment thereof may comprise the CDR sequences as defined by SEQ ID NO: 6, SEQ ID NO: 7 and SEQ ID NO: 8 and the remainder of the light chain sequence may have at least 85% sequence identity to SEQ ID NO: 12. Suitably, a unit dosage form is provided in which the heavy chain of the anti-CD38 antibody or antigen binding fragment thereof may comprise the CDR sequences as defined by SEQ ID NO: 3, SEQ ID NO: 4 and SEQ ID NO: 5 and the remainder of the heavy chain sequence may have at least 90% sequence identity to SEQ ID NO: 11 and the light chain of the anti-CD38 antibody or antigen binding fragment thereof may comprise the CDR sequences as defined by SEQ ID NO: 6, SEQ ID NO: 7 and SEQ ID NO: 8 and the remainder of the light chain sequence may have at least 90% sequence identity to SEQ ID NO: 12. Suitably, a unit dosage form is provided in which the heavy chain of the anti-CD38 antibody or antigen binding fragment thereof may comprise the CDR sequences as defined by SEQ ID NO: 3, SEQ ID NO: 4 and SEQ ID NO: 5 and the remainder of the heavy chain sequence may have at least 95% sequence identity to SEQ ID NO: 11 and the light chain of the anti-CD38 antibody or antigen binding fragment thereof may comprise the CDR sequences as defined by SEQ ID NO: 6, SEQ ID NO: 7 and SEQ ID NO: 8 and the remainder of the light chain sequence may have at least 95% sequence identity to SEQ ID NO: 12. Suitably, a unit dosage form is provided in which the heavy chain of the anti-CD38 antibody or antigen binding fragment thereof may comprise the CDR sequences as defined by SEQ ID NO: 3, SEQ ID NO: 4 and SEQ ID NO: 5 and the remainder of the heavy chain sequence may have at least 97% sequence identity to SEQ ID NO: 11 and the light chain of the anti-CD38 antibody or antigen binding fragment thereof may comprise the CDR sequences as defined by SEQ ID NO: 6, SEQ ID NO: 7 and SEQ ID NO: 8 and the remainder of the light chain sequence may have at least 97% sequence identity to SEQ ID NO: 12. Suitably, a unit dosage form is provided in which the heavy chain of the anti-CD38 antibody or antigen binding fragment thereof may comprise the CDR sequences as defined by SEQ ID NO: 3, SEQ ID NO: 4 and SEQ ID NO: 5 and the remainder of the heavy chain sequence may have at least 99% sequence identity to SEQ ID NO: 11 and the light chain of the anti-CD38 antibody or antigen binding fragment thereof may comprise the CDR sequences as defined by SEQ ID NO: 6, SEQ ID NO: 7 and SEQ ID NO: 8 and the remainder of the light chain sequence may have at least 99% sequence identity to SEQ ID NO: 12.

In one aspect, a unit dosage form is provided in which the anti-CD38 antibody or antigen binding fragment thereof comprises a heavy chain amino acid sequence of SEQ ID NO:11 or a variant thereof with up to three amino acid substitutions and a light chain amino acid sequence of SEQ ID NO:12 or a variant thereof with up to three amino acid substitutions.

In one aspect, a unit dosage form is provided in which the anti-CD38 antibody or antigen binding fragment thereof comprises a heavy chain amino acid sequence of SEQ ID NO:11 and a light chain amino acid sequence of SEQ ID NO:12.

In one aspect, a unit dosage form is provided in which the human anti-CD38 antibody or antigen binding fragment thereof is administered in the form of a pharmaceutically acceptable composition. Suitably, the pharmaceutically acceptable composition is suitable for subcutaneous administration.

In one aspect, the unit dosage form is formulated for subcutaneous administration of the antibody or antigen binding fragment thereof in the treatment of a hematological cancer selected from the group consisting of multiple myeloma, chronic lymphoblastic leukemia, chronic lymphocytic leukemia, plasma cell leukemia, acute myeloid leukemia, chronic myeloid leukemia, B-cell lymphoma, and Burkitt lymphoma.

In one aspect, the hematological cancer is multiple myeloma (MM). In one embodiment, the hematological cancer is newly diagnosed multiple myeloma (NDMM) or naïve multiple myeloma. In one embodiment, the hematological cancer is relapsed or refractory multiple myeloma (RRMM).

In one aspect, there is provided a human anti-CD38 antibody or antigen binding fragment thereof for use in therapy, wherein the antibody or antigen binding fragment thereof does not cause a significant level of red blood cell depletion and/or platelet depletion after administration and the human anti-CD38 antibody or antigen binding fragment thereof is administered subcutaneously in a dosage of about 300 milligrams. Suitably, the human anti-CD38 antibody or antigen binding fragment thereof which does not cause a significant level of red blood cell depletion and/or platelet depletion after administration may be an anti-CD38 antibody or antigen binding fragment thereof as defined herein.

In one aspect, there is provided a unit dosage form comprising an isolated antibody or antigen binding fragment thereof that does not cause a significant level of red blood cell depletion and/or platelet depletion after administration, and the unit dosage form is formulated for subcutaneous administration of the antibody or antigen binding fragment thereof at a dosage of about 300 milligrams.

In one aspect, there is provided a human anti-CD38 antibody or antigen binding fragment thereof as defined herein for use in therapy, wherein the human anti-CD38 antibody or antigen binding fragment thereof is formulated for subcutaneous administration. Suitably, the human anti-CD38 antibody or antigen binding fragment thereof is administered subcutaneously.

In one aspect, there is provided a human anti-CD38 antibody or antigen binding fragment thereof as defined herein for use in the treatment of a disease in which binding to CD38 is indicated, wherein the human anti-CD38 antibody or antigen binding fragment thereof is formulated for subcutaneous administration. Suitably, the human anti-CD38 antibody or antigen binding fragment thereof is administered subcutaneously.

In one aspect, the dosage of the administered anti-CD38 antibody or antigen binding fragment thereof and dexamethasone in combination with (a) lenolidomide, (b) lenolidomide and bortezomib, or (c) pomolidomide as described herein is a weekly dosage. In one aspect, the dosage of the administered anti-CD38 antibody or antigen binding fragment thereof as described herein is a biweekly dosage. In one aspect, the dosage of the administered anti-CD38 antibody or antigen binding fragment thereof as described herein is a once every four weeks dosage.

Suitably, the human anti-CD38 antibody or antigen binding fragment thereof may be administered in a dosage of about 300 milligrams of antibody. Suitably, the human anti-CD38 antibody or antigen binding fragment thereof may be formulated for subcutaneous administration. Suitably, the human anti-CD38 antibody or antigen binding fragment thereof may be formulated for subcutaneous administration in a dosage of about 300 milligrams of antibody.

In one aspect, there is provided a human anti-CD38 antibody or antigen binding fragment thereof as defined herein for use in the treatment of a hematological cancer wherein the human anti-CD38 antibody or antigen binding fragment thereof is formulated for subcutaneous administration and the human anti-CD38 antibody or antigen binding fragment thereof is administered in a dosage of about 300 milligrams of antibody. Suitably, the human anti-CD38 antibody or antigen binding fragment thereof may be administered subcutaneously.

Suitably, the hematological cancer may be multiple myeloma, chronic lymphoblastic leukemia, chronic lymphocytic leukemia, plasma cell leukemia, acute myeloid leukemia, chronic myeloid leukemia, B-cell lymphoma, or Burkitt lymphoma.

In one embodiment, the hematological cancer is multiple myeloma (MM). In one embodiment, the hematological cancer is newly diagnosed multiple myeloma (NDMM) or naïve multiple myeloma. In one embodiment, the hematological cancer is relapsed or refractory multiple myeloma (RRMM).

These and other embodiments, features and potential advantages will become apparent with reference to the following description.

### DETAILED DESCRIPTION OF THE INVENTION

### Introduction

There are approximately ˜36-fold more CD38 molecules expressed on RBCs than on myeloma cells in the vasculature of patients with active disease. Thus, for example, off-target expression of CD38 may need to be saturated before unbound antibody can pass into the bone marrow and saturate CD38 expressed on myeloma cells. This could explain why other anti-CD38 antibodies in the art, such as daratumumab and isatuximab, which strongly bind to RBCs and platelets, require high dose systemic administration to achieve efficacy.

AB79, daratumumab, isatuximab, and MOR202 are anti-CD38 IgG1 antibodies that primarily kill tumors by antibody-dependent cellular cytotoxicity (ADCC). This mechanism requires effector cells, such as NK cells, to bind antibodies on target cells and form a lytic synapse to secrete cytotoxic agents in a focused manner. The frequency of these effector cells in blood is orders of magnitude lower than that of RBCs and platelets. For example, the ratio of RBCs to NK cells in blood is 20,000:1. Consequently, effector activity for daratumumab, isatuximab and MOR202 is diverted from tumors because the effector cells are primarily bound by those anti-CD-38 antibodies bound to RBCs and platelets, preventing the formation of a lytic synapse with tumors, which results in a low efficiency of ADCC.

Treatment of patients with anti-CD38 antibodies that bind to RBCs and platelets may result in life threatening side effects. For example, in one study, treatment of relapsed or refractory multiple myeloma with MOR202 resulted in several serious treatment-related adverse events or TEAEs (see, e.g., Raab et al. (2015) Blood 126: 3035). The most common TEAEs at any grade were anemia (15 patients, 34%), fatigue (14 patients, 32%), infusion-related reactions (IRRs) and leukopenia (13 patients, 30% each), lymphopenia and nausea (11 patients, 25% each). Grade ≥3 TEAEs were reported for 28 patients (64%); the most common included lymphopenia (8 patients, 18%), leukopenia (5 patients, 11%) and hypertension (4 patients, 9%). IRRs arose mainly during the first infusion; all were grade 1-2 except for one patient (grade 3). Infections were commonly reported (26 patients, 59%) but in the majority of the cases were not considered to be treatment-related. MOR202 has only been used clinically via IV infusion.

Other Morphosys antibodies targeting CD38 are known (see, e.g., WO 2006/125640, which discloses four human antibodies: MOR03077, MOR03079, MOR03080, and MOR03100 and two murine antibodies: OKT10 and IB4). These prior art antibodies are inferior to antibodies for use according to the present invention (e.g., AB79) for a variety of reasons. MOR03080 binds to human CD38 and cynomolgus CD38 but with a low affinity to human CD38 (Biacore K_{D}=27.5 nm). OKT10 binds to human CD38 and cynomolgus CD38 but with a low/moderate affinity to human CD38 (Biacore K_{D}=8.28 nm). MOR03079 binds to human CD38 with a high affinity (Biacore K_{D}=2.4 nm) but does not bind to cynomolgus CD38. MOR03100 and MOR03077 bind to human CD38 with moderate or low affinity (Biacore K_{D}=10 nm and 56 nm, respectively). By comparison, antibodies for use according to the present invention (e.g., AB79) binds to human and cynomolgus CD38 with a high affinity to human CD38 (Biacore K_{D}=5.4 nm). Moreover, the prior art antibodies have poor ADCC as well as CDC activity.

An advantage of more efficient ADCC is the ability to deliver an anti-CD38 therapeutic as a low volume injection. If an antibody for use according to the present invention (e.g., AB79) is formulated at a concentration of 100 mg/mL, an efficacious dose for an 80 kg myeloma patient could be administered as a single s.c. injection of <1.0 mL. In contrast, an effective dose of daratumumab or isatuximab delivered into this patient with a comparable form (i.e., 100 mg/mL) would require administering 12.8 mL or 8-16 mL, respectively.

The anti-CD38 methods and unit dosages provide herein subcutaneous administration of therapeutically effective doses of anti-CD38 antibodies or antigen binding fragments thereof in combination with (a) lenolidomide, (b) lenolidomide and bortezomib, or (c) pomolidomide, thereby providing unexpected benefits and preventing the side effects, inconvenience, and expense of administering high dose, systemic anti-CD38 antibody therapies.

The present invention provides methods and unit dosage forms for subcutaneous administration of a therapeutically effective amount of an isolated anti-CD38 antibody or antigen binding fragment thereof to a patient in need thereof to treat diseases in which binding to CD38 is indicated, including hematological cancers. In some embodiments, the antibody or antigen binding fragment thereof for subcutaneous administration comprises a heavy chain variable region comprising SEQ ID NO:9 (or a sequence with at least 80%, 85%, 90%, 95%, 97% or 99% sequence identity thereto) and a light chain variable region comprising SEQ ID NO:10 (or a sequence with at least 80%, 85%, 90%, 95%, 97% or 99% sequence identity thereto). The anti-CD38 antibody or antigen binding fragment thereof provided herein is capable of being therapeutically effective when administered by subcutaneous administration.

Lenalidomide (LEN) is currently marketed as Revlimid by Celgene for the treatment of multiple myeloma. Lenalidomide is cytotoxic to tumor cells, activates natural killer (NK) cells, and upregulates CD38 expression on tumor cells. Lenalidomide is a thalidomide analog, therefore, it is expected that other thalidomide analogs such as pomalidomide or thalidomide itself may be efficatious when used in combination with an anti-CD38 antibody or antigen binding fragment thereof of the invention.

Pomalidomide (POM) is currently marketed as Pomalyst in the U.S. by Celgene and Imnovid in the EU and Russia by Celgene.

Dexamethasone (DEX) is a corticosteroid that synergizes with lenalidomide and pomalidomide to inhibit MM tumor growth. It is used in the treatment of many conditions including as an anti-inflammatory and immunosuppressant and is used in cancer treatment to counteract certain side effects of the anti-tumor treatment.

Bortezomib (originally PS-341; marketed as Velcade (VEL) by Takeda Oncology; Chemobort by Cytogen and Bortecad by Cadila Healthcare) is a chemotherapeutic agent of the peptide boronate class that acts as a proteasome inhibitor. Several other classes of proteasome inhibitors are known. Peptide boronates are approved in the U.S. for the treatment of relapsed multiple myeloma. Another peptide boronate is CEP-18770. Other classes of proteasome inhibitors include peptide aldehydes (e.g., MG132), peptide vinyl sulfones, peptide epoxyketones (e.g., epoxomicin, carfilzomib), β lactone inhibitors (e.g., lactacystin, MLN 519, NPI-0052, Salinosporamide A), compounds which create dithiocarbamate complexes with metals (e.g., disulfuram) and certain antioxidants (e.g., epigallocatechin-3-gallate) catechin-3-gallate, and salinosporamide A. Another proteasome inhibitor, ixazomib, was approved by the FDA in 2015 for use in combination with lenalidomide and dexamethasone for the treatment of multiple myeloma after at least one prior therapy.

Unless otherwise defined herein, scientific and technical terms used in connection with the present invention shall have the meanings that are commonly understood by those of ordinary skill in the art. The meaning and scope of the terms should be clear. However, in the event of any latent ambiguity, definitions provided herein take precedence over any dictionary or extrinsic definition. Further, unless otherwise required by context, singular terms shall include pluralities and plural terms shall include the singular. The term “or” includes “and/or” unless stated otherwise. Furthermore, the use of the term “including,” “includes,” or “included” is not limiting. Terms such as “element” and “component” encompass both elements and components comprising one unit and elements and components that comprise more than one subunit unless specifically stated otherwise.

Generally, nomenclature used in connection with, and techniques of, cell and tissue culture, molecular biology, immunology, microbiology, genetics and protein and nucleic acid chemistry and hybridization described herein are well-known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are well-known and commonly used in the art. Standard techniques are used for chemical syntheses, chemical analyses, pharmaceutical preparation, formulation, delivery, and treatment of patients.

All headings and section designations are used for clarity and reference purposes only and are not to be considered limiting in any way. For example, those of skill in the art will appreciate the usefulness of combining various aspects of the disclosure from different headings and sections as appropriate according to the spirit and scope of the invention described herein.

### Definitions

Select terms are defined below in order for the present invention to be more readily understood.

The terms “human CD38” and “human CD38 antigen” refer to the amino acid sequence of SEQ ID NO:1, or a functional fraction thereof, such as an epitope, as defined herein (Table 1). In general, CD38 possesses a short intracytoplasmic tail, a transmembrane domain, and an extracellular domain. The terms “cynomolgus CD38” and “cynomolgus CD38 antigen” refer to the amino acid sequence of SEQ ID NO:2, which is 92% identical to the amino acid sequence of human CD38 (Table 1). Synonyms for CD38 include cyclic ADP ribose hydrolase; cyclic ADP ribose-hydrolase 1; ADP ribosyl cyclase; ADP-ribosyl cyclase 1; cADPr hydrolase 1; CD38-rs1; I-19; NIM-R5 antigen; 2′-phospho-cyclic-ADP-ribose transferase; 2′-phospho-ADP-ribosyl cyclase; 2′-phospho-cyclic-ADP-ribose transferase; 2′-phospho-ADP-ribosyl cyclase; T10.

TABLE 1
Amino Acid Sequence of Human and
Cynomolgus Monkey CD38

| | | SEQ |
|---|---|---|
| | | ID |
| Species | Amino Acid Sequence | NO |
| Human | MANCEFSPVSGDKPCCRLSRRAQLCLGVSILVLILVVVL | 1 |
| CD38 | AVVVPRWRQQWSGPGTTKRFPETVLARCVKYTEIHPE | |
| | MRHVDCQSVWDAFKGAFISKHPCNITEEDYQPLMKLG | |
| | TQTVPCNKILLWSRIKDLAHQFTQVQRDMFTLEDTLLG | |
| | YLADDLTWCGEFNTSKINYQSCPDWRKDCSNNPVSVF | |
| | WKTVSRRFAEAACDVVHVMLNGSRSKIFDKNSTFGSVE | |
| | VHNLQPEKVQTLEAWVIHGGREDSRDLCQDPTIKELESI | |
| | ISKRNIQFSCKNIYRPDKFLQCVKNPEDSSCTSEI | |
| Cyno | MANCEFSPVSGDKPCCRLSRRAQVCLGVCLLVLLILVV | 2 |
| CD38 | VVAVVLPRWRQQWSGSGTTSRFPETVLARCVKYTEVH | |
| | PEMRHVDCQSVWDAFKGAFISKYPCNITEEDYQPLVKL | |
| | GTQTVPCNKTLLWSRIKDLAHQFTQVQRDMFTLEDML | |
| | LGYLADDLTWCGEFNTFEINYQSCPDWRKDCSNNPVSV | |
| | FWKTVSRRFAETACGVVHVMLNGSRSKIFDKNSTFGSV | |
| | EVHNLQPEKVQALEAWVIHGGREDSRDLCQDPTIKELE | |
| | SIISKRNIRFFCKNIYRPDKFLQCVKNPEDSSCLSGI | |
| Human | MAAQGCAASRLLQLLLQLLLLLLLLAAGGARARWRGE | 13 |
| CD157 | GTSAHLRDIFLGRCAEYRALLSPEQRNKNCTAIWEAFK | |
| | VALDKDPCSVLPSDYDLFINLSRHSIPRDKSLFWENSHL | |
| | LVNSFADNTRRFMPLSDVLYGRVADFLSWCRQKNDSG | |
| | LDYQSCPTSEDCENNPVDSFWKRASIQYSKDSSGVIHV | |
| | MLNGSEPTGAYPIKGFFADYEIPNLQKEKITRIEIWVMH | |
| | EIGGPNVESCGEGSMKVLEKRLKDMGFQYSCINDYRPV | |
| | KLLQCVDHSTHPDCALKSAAAATQRKAPSLYTEQRAG | |
| | LIIPLFLVLASRTQL | |

The terms “therapeutically effective amount” and “therapeutically effective dosage” refer to an amount of a therapy that is sufficient to reduce or ameliorate the severity and/or duration of a disorder or one or more symptoms thereof; prevent the advancement of a disorder; cause regression of a disorder; prevent the recurrence, development, onset, or progression of one or more symptoms associated with a disorder; or enhance or improve the prophylactic or therapeutic effect(s) of another therapy (e.g., prophylactic or therapeutic agent), at dosages and for periods of time necessary to achieve a desired therapeutic result. A therapeutically effective amount may vary according to factors such as the disease state, age, sex, and weight of the individual, and the ability of the medicaments to elicit a desired response in the individual. A therapeutically effective amount of an antibody is one in which any toxic or detrimental effects of the antibody or antibody portion are outweighed by the therapeutically beneficial effects. A therapeutically effective amount of an antibody for tumor therapy may be measured by its ability to stabilize the progression of disease. The ability of a compound to inhibit cancer may be evaluated in an animal model system predictive of efficacy in human tumors. The terms “unit dose” or “dosage form” are the amount of a medication administered to a patient in a single dose. Dosage forms are pharmaceutical drug products in the form in which they are marketed for use, with a specific mixture of active ingredients and inactive components (excipients), in a particular configuration (such as a capsule shell, for example), and apportioned into a particular dose.

The terms “patient” and “subject” include both humans and other animals, particularly mammals. Thus, the compositions, dosages, and methods disclosed herein are applicable to both human and veterinary therapies. In one embodiment, the patient is a mammal, for example, a human.

The term “disease in which binding to CD38 is indicated” means a disease in which binding of a binding partner (e.g., an anti-CD38 antibody of the invention) to CD38 provides a prophylactic or curative effect, including the amelioration of one or more symptoms of the disease. Such binding could result in the blocking of other factors or binding partners for CD38, neutralization of CD38, ADCC, CDC, complement activation, or some other mechanism by which the disease is prevented or treated. Factors and binding partners for CD38 include autoantibodies to CD38, which are blocked by the anti-CD38 antibodies or antigen binding fragments thereof of the invention. Such binding may be indicated as a consequence of expression of CD38 by cells or a subset of cells, e.g., MM cells, by which providing a binding partner of CD38 to the subject results in the removal, e.g., lysis, of those cells, e.g., via hemolysis or apoptosis. Such expression of CD38 may be, e.g., normal, overexpressed, inappropriately expressed, or a consequence of activation of CD38, relative to normal cells or relative to other cells types either during a non-disease state or a disease state.

The term “hematologic cancer” refers to malignant neoplasms of blood-forming tissues and encompasses leukemias, lymphomas and multiple myelomas. Non-limiting examples of conditions associated with aberrant CD38 expression include, but are not limited to, multiple myeloma; B-cell chronic lymphocytic leukemia (B-CLL); acute lymphoblastic leukemia; chronic myeloid leukemia; acute myeloid leukemia; chronic lymphocytic leukemia (CLL); chronic myelogenous leukemia or chronic myeloid leukemia (CIVIL); acute myelogenous leukemia or acute myeloid leukemia (AML); acute lymphocytic leukemia (ALL); hairy cell leukemia (HCL); myelodysplastic syndromes (MDS); and all subtypes and stages (e.g., CIVIL blastic phase (BP), chronic phase (CP), or accelerated phase (AP)) of these leukemias and other hematologic diseases, which are defined by morphological, histochemical and immunological techniques that are well known to those of skill in the art.

The term “isolated antibody” refers to an antibody or antigen binding fragment thereof that is substantially free of other antibodies having different antigenic specificities. For instance, an isolated antibody that specifically binds to CD38 is substantially free of antibodies that specifically bind antigens other than CD38. An isolated antibody that specifically binds to an epitope, isoform or variant of human CD38 or cynomolgus CD38 may, however, have cross-reactivity to other related antigens, for instance from other species, such as CD38 species homologs. Moreover, an isolated antibody may be substantially free of other cellular material and/or chemicals or to a homogenous population of antibodies which have been substantially separated and/or purified away from other components of the system from which the antibody was produced, such as a recombinant cell.

The term “recombinant antibody” refers to antibodies that are prepared, expressed, created, or isolated by recombinant means such as, e.g., antibodies isolated from an animal (e.g., a mouse) that is transgenic or transchromosomal for human immunoglobulin genes or a hybridoma prepared therefrom; antibodies isolated from a host cell transformed to express the antibody; antibodies isolated from a combinatorial antibody library; and antibodies created by any other means that involve splicing of human immunoglobulin gene sequences to other DNA sequences, or antibodies that are generated in vitro.

The terms “red blood cells,” “RBCs,” and “erythrocytes” refer to bone marrow derived hemoglobin-containing blood cells that carry oxygen to cells and tissues and carry carbon dioxide back to respiratory organs. RBCs are also referred to as red cells, red blood corpuscles, haematids, and erythroid cells.

The term “over a period of time” refers to any period of time, e.g., minutes, hours, days, months, or years. For example, over a period of time can refer to at least 10 minutes, at least 15 minutes, at least 30 minutes, at least 60 minutes, at least 75 minutes, at least 90 minutes, at least 105 minutes, at least 120 minutes, at least 3 hours, at least 4 hours, at least 5 hours, at least 6 hours, at least 7 hours, at least 8 hours, at least 9 hours, at least 10 hours, at least 12 hours, at least 14 hours, at least 16, hours, at least 18 hours, at least 20 hours, at least 22 hours, at least one day, at least two days, at least three days, at least 4 days, at least 5 days, at least 6 days, at least a week, at least on month, at least one year, or any interval of time in between. In other words, the antibody from the composition can be absorbed by the individual to whom it is administered over a period of at least 10 minutes, at least 15 minutes, at least 30 minutes, at least 60 minutes, at least 75 minutes, at least 90 minutes, at least 105 minutes, at least 120 minutes, at least 3 hours, at least 4 hours, at least 5 hours, at least 6 hours, at least 7 hours, at least 8 hours, at least 9 hours, at least 10 hours, at least 12 hours, at least 14 hours, at least 16, hours, at least 18 hours, at least 20 hours, at least 22 hours, at least one day, at least two days, at least three days, at least 4 days, at least 5 days, at least 6 days, at least a week, at least on month, at least one year, or any interval of time in between.

The term “treatment cycle” refers to a period of treatment with a drug or combination of drugs, followed by a rest period (i.e., no treatment) with one or more of the drugs. A typical treatment cycle would be 28 days but may vary. The cycle can be repeated multiple times on a regular schedule to make up a full course of treatment. A course of treatment may be between 4 and 8 cycles and may be cut short or extended depending upon the patient's reaction.

A composition that “substantially” comprises a component means that the composition contains more than about 80% by weight of the component. Suitably, the composition may comprise more than about 90% by weight of the component. Suitably, the composition may comprise more than about 95% by weight of the component. Suitably, the composition may comprise more than about 97% by weight of the component. Suitably, the composition may comprise more than about 98% by weight of the component. Suitably the composition may comprise more than about 99% by weight of the component.

The term “about” refers to an extent near in number, degree, volume, time, etc., with only minor variations in dimension of up to 10%. Thus, the term “about” is used to encompass variations of ±10% or less, variations of ±5% or less, variations of ±1% or less, variations of ±0.5% or less, or variations of ±0.1% or less from the specified value.

The term “pharmaceutically acceptable carrier” refers to a pharmaceutically acceptable material, composition or vehicle, suitable for administering compounds of the present invention to mammals. The carriers include liquid or solid filler, diluent, excipient, solvent or encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. In one embodiment, the pharmaceutically acceptable carrier is suitable for intravenous administration. In another embodiment, the pharmaceutically acceptable carrier is suitable for locoregional injection. In another embodiment, the pharmaceutically acceptable carrier is suitable for subcutaneous administration. In another embodiment, the pharmaceutically acceptable carrier is suitable for subcutaneous injection.

The term “pharmaceutical composition” refers to preparations suitable for administration to a subject and treatment of disease. When the anti-CD38 antibodies or antigen binding fragments thereof of the present invention are administered as pharmaceuticals to mammals, e.g., humans, they can be administered “as is” or as a pharmaceutical composition containing the anti-CD38 antibody or antigen binding fragment thereof in combination with a pharmaceutically acceptable carrier and/or other excipients. The pharmaceutical composition can be in the form of a unit dosage form for administration of a particular dosage of the anti-CD38 antibody or antigen binding fragment thereof at a particular concentration, a particular amount, or a particular volume. Pharmaceutical compositions comprising the anti-CD38 antibodies or antigen binding fragments thereof, either alone or in combination with prophylactic agents, therapeutic agents, and/or pharmaceutically acceptable carriers are provided. Suitably, the pharmaceutical composition may comprise a unit dosage form according to the present invention either alone or in combination with prophylactic agents, therapeutic agents, and/or pharmaceutically acceptable carriers. Suitably, the pharmaceutical composition may comprise a human anti-CD38 antibody or antigen binding fragment thereof as described herein either alone or in combination with prophylactic agents, therapeutic agents, and/or pharmaceutically acceptable carriers.

“In combination with” means that two or more therapeutics can be administered to a subject together in a mixture, concurrently as single agents, or sequentially as single agents in any order. The mode of administration of each therapeutic may vary, e.g., in a triple combination therapy, one therapeutic may be administered subcutaneously, one may be administered orally, and one may be administered intravenously.

Traditional antibody structural units typically comprise a tetramer. Each tetramer is typically composed of two identical pairs of polypeptide chains, each pair having one “light” chain (typically having a molecular weight of about 25 kDa) and one “heavy” chain (typically having a molecular weight of about 50-70 kDa). Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Thus, “isotype” refers to any of the subclasses of immunoglobulins defined by the chemical and antigenic characteristics of their constant regions. The known human immunoglobulin isotypes are IgG1, IgG2, IgG3, IgG4, IgA1, IgA2, IgM1, IgM2, IgD, and IgE. Therapeutic antibodies can also comprise hybrids of isotypes and/or subclasses.

Each variable heavy (VH) and variable light (VL) region (about 100 to 110 amino acids in length) is composed of three hypervariable regions called “complementarity determining regions” (CDRs) and four framework regions (FRs) (about 15-30 amino acids in length), arranged from amino-terminus to carboxy-terminus in the following order: FR1-CDR1-FR2-CDR2-FR3-CDR3-FR4. “Variable” refers to the fact that the CDRs differ extensively in sequence among antibodies and thereby determines a unique antigen binding site.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region (Kabat et al. (1991) Sequences Of Proteins Of Immunological Interest, 5th Ed. Public Health Service, National Institutes of Health, Bethesda, Md.) and/or those residues forming a hypervariable loop (e.g., residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region (Chothia and Lesk (1987) J. Mol. Biol. 196: 901-917.

The Kabat numbering system is generally used when referring to a residue in the variable domain (approximately, residues 1-107 of the light chain variable region and residues 1-113 of the heavy chain variable region) (e.g., Kabat et al. (1991) Sequences Of Proteins Of Immunological Interest, 5^{th} Ed. Public Health Service, National Institutes of Health, Bethesda, Md.), with the EU number system used for the Fc region.

The term “immunoglobulin (Ig) domain” refers to a region of an immunoglobulin having a distinct tertiary structure. In addition to the variable domains, each heavy and light chain has constant domains: constant heavy (CH) domains; constant light (CL) domains and hinge domains. In the context of IgG antibodies, the IgG isotypes each have three CH regions. The carboxy-terminal portion of each HC and LC defines a constant region primarily responsible for effector function. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat.

The term “hinge region” refers to the flexible polypeptide comprising the amino acids between the first and second constant domains of an antibody. Structurally, the IgG CH1 domain ends at EU position 220, and the IgG CH2 domain begins at residue EU position 237. Thus, for IgG the antibody hinge is herein defined to include positions 221 (D221 in IgG1) to 236 (G236 in IgG1), wherein the numbering is according to the EU index as in Kabat. In some embodiments, for example in the context of an Fc region, the lower hinge is included, with the “lower hinge” generally referring to positions 226 to 230.

The term “Fc region” refers to the polypeptide comprising the constant region of an antibody excluding the first constant region Ig domain and in some cases, part of the hinge. Thus, Fc refers to the last two constant region Ig domains of IgA, IgD, and IgG, the last three constant region Ig domains of IgE and IgM, and the flexible hinge N-terminal to these domains. For IgA and IgM, Fc may include the J chain. For IgG, the Fc domain comprises Ig domains Cγ2 and Cγ3 (Cγ2 and Cγ3) and the lower hinge region between Cγ1 (Cγ1) and Cγ2 (Cγ2). Although the boundaries of the Fc region may vary, the human IgG heavy chain Fc region is usually defined to include residues C226 or P230 to its carboxyl-terminus, wherein the numbering is according to the EU index as in Kabat. In some embodiments, as is more fully described below, amino acid modifications are made to the Fc region, for example to alter binding to one or more FcγR receptors or to the FcRn receptor.

The term “humanized antibody” refers to an antibody in which the antigen binding sites are derived from antibody sequences from a non-human species and the framework and constant regions are derived from human antibody sequences. Humanized antibodies may include substitutions in the framework regions so that the framework may not be an exact copy of expressed human antibody or germline gene sequences. The term “derived from” in reference to humanized antibodies, means that the Ig domain in question is at least 80% identical to the sequence of the antibody from the species to which it refers.

The term “human antibody” refers to an antibody in which both the antigen binding sites, framework regions, and constant regions are derived from sequences of human origin, e.g., they are “derived from” sequences of human origin if the variable regions of the antibody are obtained from a system that uses human germline immunoglobulin or rearranged immunoglobulin genes. Such systems include human immunoglobulin gene libraries displayed on phage, and transgenic non-human animals such as mice carrying human immunoglobulin loci as described herein. “Human antibody” may contain amino acid differences when compared to the human germline or rearranged immunoglobulin sequences due to, e.g., naturally occurring somatic mutations or intentional introduction of substitutions in the framework or antigen binding sites. Typically, a “human antibody” is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical in amino acid sequence to an amino acid sequence encoded by a human germline or rearranged immunoglobulin gene.

### CD38 Antibodies

Accordingly, the present invention provides isolated anti-CD38 antibodies and antigen binding fragments thereof that specifically bind human and primate CD38 protein that find use in subcutaneous administration methods and unit dosage forms. Of particular use in the present invention are antibodies or antigen binding fragments thereof that bind to both the human and primate CD38 proteins, particularly primates used in clinical testing, such as cynomolgus monkeys (Macaca fascicularis, Crab eating macaque, also referred to herein as “cyno”).

In some embodiments, the anti-CD38 antibodies or antigen binding fragments thereof of the invention interact with CD38 at a number of amino acid residues including K121, F135, Q139, D141, M142, E239, W241, S274, C275, K276, F284, V288, K289, N290, P291, E292, D293 and S294 based on human sequence numbering. Suitably, the anti-CD38 antibodies or antigen binding fragments thereof of the invention may interact with CD38 at a number of amino acid residues including K121, F135, Q139, D141, M142, E239, W241, S274, C275, K276, F284, V288, K289, N290, P291, E292, D293 and S294 of SEQ ID NO: 1, based on human sequence numbering. Suitably, the anti-CD38 antibodies or antigen binding fragments thereof of the invention interact with CD38 at a number of amino acid residues including K121, F135, Q139, D141, M142, E239, W241, F274, C275, K276, F284, V288, K289, N290, P291, E292, D293 and S294 of SEQ ID NO: 2. It should be noted that these residues are identical in both human and cynomolgus monkeys, with the exception that S274 is actually F274 in cynomolgus monkeys. These residues may represent the immunodominant epitope and/or residues within the footprint of the specific antigen binding peptide.

In some embodiments, the anti-CD38 antibody or antigen binding fragment thereof for use according to the invention comprises a heavy chain comprising the following CDR amino acid sequences: GFTFDDYG (SEQ ID NO:3; HCDR1 AB79), ISWNGGKT (SEQ ID NO:4; HCDR2 AB79), and ARGSLFHDSSGFYFGH (SEQ ID NO:5; HCDR3 AB79) or variants of those sequences having up to three amino acid changes. In some embodiments, the antibody or antigen binding fragment thereof for use according to the invention comprises a light chain comprising the following CDR amino acid sequences: SSNIGDNY (SEQ ID NO:6; LCDR1 AB79), RDS (SEQ ID NO:7; LCDR2 AB79), and QSYDSSLSGS (SEQ ID NO:8; LCDR3 AB79) or variants of those sequences having up to three amino acid changes. In some embodiments, the antibody or antigen binding fragment thereof for use according to the invention comprises a heavy chain comprising the following CDR amino acid sequences: GFTFDDYG (SEQ ID NO:3; HCDR1 AB79), ISWNGGKT (SEQ ID NO:4; HCDR2 AB79), ARGSLFHDSSGFYFGH (SEQ ID NO:5; HCDR3 AB79) or variants of those sequences having up to three amino acid changes and a light chain comprising the following CDR amino acid sequences: SSNIGDNY (SEQ ID NO:6; LCDR1 AB79), RDS (SEQ ID NO:7; LCDR2 AB79), and QSYDSSLSGS (SEQ ID NO:8; LCDR3 AB79) or variants of those sequences having up to three amino acid changes. In some embodiments, the anti-CD38 antibody or antigen binding fragment thereof comprises a heavy chain comprising the following CDR amino acid sequences: GFTFDDYG (SEQ ID NO:3; HCDR1 AB79), ISWNGGKT (SEQ ID NO:4; HCDR2 AB79), and ARGSLFHDSSGFYFGH (SEQ ID NO:5; HCDR3 AB79). In some embodiments, the antibody or antigen binding fragment thereof comprises a light chain comprising the following CDR amino acid sequences: SSNIGDNY (SEQ ID NO:6; LCDR1 AB79), RDS (SEQ ID NO:7; LCDR2 AB79), and QSYDSSLSGS (SEQ ID NO:8; LCDR3 AB79). In some embodiments, the antibody or antigen binding fragment thereof comprises a heavy chain comprising the following CDR amino acid sequences: GFTFDDYG (SEQ ID NO:3; HCDR1 AB79), ISWNGGKT (SEQ ID NO:4; HCDR2 AB79), ARGSLFHDSSGFYFGH (SEQ ID NO:5; HCDR3 AB79) and a light chain comprising the following CDR amino acid sequences: SSNIGDNY (SEQ ID NO:6; LCDR1 AB79), RDS (SEQ ID NO:7; LCDR2 AB79), and QSYDSSLSGS (SEQ ID NO:8; LCDR3 AB79). In some embodiments, the antibody or antigen binding fragment thereof comprises a heavy chain comprising an amino acid sequence having at least 80% sequence identity to SEQ ID NO:9. Suitably, the VH chain may comprise the CDR sequences as defined by SEQ ID NO: 3, SEQ ID NO: 4 and SEQ ID NO: 5 and the remainder of the sequence may have at least 80% sequence identity to SEQ ID NO: 9. Suitably, the VH chain may comprise the CDR sequences as defined by SEQ ID NO: 3, SEQ ID NO: 4 and SEQ ID NO: 5 and the remainder of the sequence may have at least 85% sequence identity to SEQ ID NO: 9. Suitably, the VH chain may comprise the CDR sequences as defined by SEQ ID NO: 3, SEQ ID NO: 4 and SEQ ID NO: 5 and the remainder of the sequence may have at least 90% sequence identity to SEQ ID NO: 9. Suitably, the VH chain may comprise the CDR sequences as defined by SEQ ID NO: 3, SEQ ID NO: 4 and SEQ ID NO: 5 and the remainder of the sequence may have at least 95% sequence identity to SEQ ID NO: 9. Suitably, the VH chain may comprise the CDR sequences as defined by SEQ ID NO: 3, SEQ ID NO: 4 and SEQ ID NO: 5 and the remainder of the sequence may have at least 97% sequence identity to SEQ ID NO: 9. Suitably, the VH chain may comprise the CDR sequences as defined by SEQ ID NO: 3, SEQ ID NO: 4 and SEQ ID NO: 5 and the remainder of the sequence may have at least 99% sequence identity to SEQ ID NO: 9.

In some embodiments, the antibody or antigen binding fragment thereof comprises a heavy chain comprising the variable heavy (VH) chain amino acid sequence of SEQ ID NO:9.

(SEQ ID NO: 9)
EVQLLESGGGLVQPGGSLRLSCAASGFTFDDYGMSWVRQAPGKGLEWVSDI
SWNGGKTHYVDSVKGQFTISRDNSKNTLYLQMNSLRAEDTAVYYCARGSLF
HDSSGFYFGHWGQGTLVTVSSASTKGPSVFPLA.

In some embodiments, the antibody or antigen binding fragment thereof comprises a light chain comprising an amino acid sequence having at least 80% sequence identity to SEQ ID NO:10. Suitably, the VL chain may comprise the CDR sequences as defined by SEQ ID NO: 6, SEQ ID NO: 7 and SEQ ID NO: 8 and the remainder of the VL sequence may have at least 80% sequence identity to SEQ ID NO: 10. Suitably, the VL chain may comprise the CDR sequences as defined by SEQ ID NO: 6, SEQ ID NO: 7 and SEQ ID NO: 8 and the remainder of the VL sequence may have at least 85% sequence identity to SEQ ID NO: 10. Suitably, the VL chain may comprise the CDR sequences as defined by SEQ ID NO: 6, SEQ ID NO: 7 and SEQ ID NO: 8 and the remainder of the VL sequence may have at least 90% sequence identity to SEQ ID NO: 10. Suitably, the VL chain may comprise the CDR sequences as defined by SEQ ID NO: 6, SEQ ID NO: 7 and SEQ ID NO: 8 and the remainder of the VL sequence may have at least 95% sequence identity to SEQ ID NO: 10. Suitably, the VL chain may comprise the CDR sequences as defined by SEQ ID NO: 6, SEQ ID NO: 7 and SEQ ID NO: 8 and the remainder of the VL sequence may have at least 97% sequence identity to SEQ ID NO: 10. Suitably, the VL chain may comprise the CDR sequences as defined by SEQ ID NO: 6, SEQ ID NO: 7 and SEQ ID NO: 8 and the remainder of the VL sequence may have at least 99% sequence identity to SEQ ID NO: 10.

In some embodiments, the antibody or antigen binding fragment thereof comprises a light chain comprising the variable light (VL) chain amino acid sequence of SEQ ID NO:10.

(SEQ ID NO: 10)
QSVLTQPPSASGTPGQRVTISCSGSSSNIGDNYVSWYQQLPGTAPKLLIYR
DSQRPSGVPDRFSGSKSGTSASLAISGLRSEDEADYYCQSYDSSLSGSVFG
GGTKLTVLGQPKANPTVTLFPPSSEEL.

In some embodiments, the antibody or antigen binding fragment thereof comprises a heavy chain comprising the VH chain amino acid sequence of SEQ ID NO:9 or a variant thereof as described herein and a light chain comprising the VL chain amino acid sequence of SEQ ID NO:10 or a variant thereof as described herein.

As will be appreciated by those in the art, the variable heavy and light chains can be joined to human IgG constant domain sequences, generally IgG1, IgG2 or IgG4.

In some embodiments, the antibody or antigen binding fragment thereof comprises a heavy chain (HC) comprising an amino acid sequence having at least 80% sequence identity to SEQ ID NO:11. Suitably, the heavy chain may comprise the CDR sequences as defined by SEQ ID NO: 3, SEQ ID NO: 4 and SEQ ID NO: 5 and the remainder of the heavy chain may have at least 80% sequence identity to SEQ ID NO 11. Suitably, the heavy chain may comprise the CDR sequences as defined by SEQ ID NO: 3, SEQ ID NO: 4 and SEQ ID NO: 5 and the remainder of the heavy chain may have at least 85% sequence identity to SEQ ID NO 11. Suitably, the heavy chain may comprise the CDR sequences as defined by SEQ ID NO: 3, SEQ ID NO: 4 and SEQ ID NO: 5 and the remainder of the heavy chain may have at least 90% sequence identity to SEQ ID NO 11. Suitably, the heavy chain may comprise the CDR sequences as defined by SEQ ID NO: 3, SEQ ID NO: 4 and SEQ ID NO: 5 and the remainder of the heavy chain may have at least 95% sequence identity to SEQ ID NO 11. Suitably, the heavy chain may comprise the CDR sequences as defined by SEQ ID NO: 3, SEQ ID NO: 4 and SEQ ID NO: 5 and the remainder of the heavy chain may have at least 97% sequence identity to SEQ ID NO 11. Suitably, the heavy chain may comprise the CDR sequences as defined by SEQ ID NO: 3, SEQ ID NO: 4 and SEQ ID NO: 5 and the remainder of the heavy chain may have at least 99% sequence identity to SEQ ID NO 11.

In some embodiments, the antibody or antigen binding fragment thereof comprises the heavy chain (HC) amino acid sequence of SEQ ID NO:11.

(SEQ ID NO: 11)
EVQLLESGGGLVQPGGSLRLSCAASGFTFDDYGMSWVRQAPGKGLEWVSDI
SWNGGKTHYVDSVKGQFTISRDNSKNTLYLQMNSLRAEDTAVYYCARGSLF
HDSSGFYFGHWGQGTLVTVSSASTKGPSVFPLAPSSKSTSGGTAALGCLVK
DYFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLEPPKPKD
TLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPGK.

In some embodiments, the antibody or antigen binding fragment thereof comprises a light chain (LC) comprising an amino acid sequence having at least 80% sequence identity to SEQ ID NO:12. Suitably, the light chain may comprise the CDR sequences as defined by SEQ ID NO: 6, SEQ ID NO: 7 and SEQ ID NO: 8 and the remainder of the light chain may have at least 80% sequence identity to SEQ ID NO 12. Suitably, the light chain may comprise the CDR sequences as defined by SEQ ID NO: 6, SEQ ID NO: 7 and SEQ ID NO: 8 and the remainder of the light chain may have at least 85% sequence identity to SEQ ID NO 12. Suitably, the light chain may comprise the CDR sequences as defined by SEQ ID NO: 6, SEQ ID NO: 7 and SEQ ID NO: 8 and the remainder of the light chain may have at least 90% sequence identity to SEQ ID NO 12. Suitably, the light chain may comprise the CDR sequences as defined by SEQ ID NO: 6, SEQ ID NO: 7 and SEQ ID NO: 8 and the remainder of the light chain may have at least 95% sequence identity to SEQ ID NO 12. Suitably, the light chain may comprise the CDR sequences as defined by SEQ ID NO: 6, SEQ ID NO: 7 and SEQ ID NO: 8 and the remainder of the light chain may have at least 97% sequence identity to SEQ ID NO 12. Suitably, the light chain may comprise the CDR sequences as defined by SEQ ID NO: 6, SEQ ID NO: 7 and SEQ ID NO: 8 and the remainder of the light chain may have at least 99% sequence identity to SEQ ID NO 12.

In some embodiments, the antibody or antigen binding fragment thereof comprises the light chain (LC) amino acid sequence of SEQ ID NO:12.

(SEQ ID NO: 12)
QSVLTQPPSASGTPGQRVTISCSGSSSNIGDNYVSWYQQLPGTAPKLLIYR
DSQRPSGVPDRFSGSKSGTSASLAISGLRSEDEADYYCQSYDSSLSGSVFG
GGTKLTVLGQPKANPTVTLFPPSSEELQANKATLVCLISDFYPGAVTVAWK
ADGSPVKAGVETTKPSKQSNNKYAASSYLSLTPEQWKSHRSYSCQVTHEGS
TVEKTVAPTECS.

In some embodiments, the antibody or antigen binding fragment thereof comprises the HC amino acid sequence of SEQ ID NO:11 or a variant thereof as described herein and the LC amino acid sequence of SEQ ID NO:12 or a variant thereof as described herein.

The present invention encompasses antibodies or antigen binding fragments thereof that bind to both human and cyno CD38 and interact with at least 80%, 85%, 90%, 91%, 92%, 93%, 94%, 95%, 96%, 97%, 98% or 99% of the following amino acid residues: K121, F135, Q139, D141, M142, E239, W241, S274, C275, K276, F284, V288, K289, N290, P291, E292, D293 and S294 of SEQ ID NO: 1 and SEQ ID NO: 2, based on human numbering. Suitably, the antibody or antigen binding fragment thereof may interact with at least 90% of these amino acid residues. Suitably, the antibody or antigen binding fragment thereof may interact with at least 95% of these amino acid residues. Suitably, the antibody or antigen binding fragment thereof may interact with at least 97% of these amino acid residues. Suitably, the antibody or antigen binding fragment thereof may interact with at least 98% of these amino acid residues. Suitably, the antibody or antigen binding fragment thereof may interact with at least 99% of these amino acid residues. Suitably, the antibody or antigen binding fragment thereof may interact with at least 14 (e.g., at least 15 or at least 16) of the following amino acids: K121, F135, Q139, D141, M142, E239, W241, S274, C275, K276, F284, V288, K289, N290, P291, E292, D293 and S294 of SEQ ID NO: 1 and SEQ ID NO: 2, based on human numbering.

In some embodiments, the antibodies are full length. By “full length antibody” herein is meant the structure that constitutes the natural biological form of an antibody, including variable and constant regions, including one or more modifications as outlined herein.

Alternatively, the antibodies can be a variety of structures, including, but not limited to, antibody fragments, antigen binding fragments, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), and fragments of each, respectively. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al. (1989) Nature 341: 544-546) which consists of a single variable, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al. (1988) Science 242: 423-426, Huston et al. (1988) Proc. Natl. Acad. Sci. USA 85: 5879-5883), (viii) bispecific single chain Fv (WO 03/11161) and (ix) “diabodies” or “triabodies”, multivalent or multispecific fragments constructed by gene fusion (Tomlinson et al. (2000) Methods Enzymol. 326: 461-479; WO94/13804; Holliger et al. (1993) Proc. Natl. Acad. Sci. USA 90: 6444-6448).

Suitably, the antibody may be a Fab fragment. Suitably, the antibody may be an Fv fragment. Suitably, the antibody may be an Fd fragment. Suitably, the antibody structure may be isolated CDR regions. Suitably, the antibody may be a F(ab′)2 fragment. Suitably, the antibody may be an scFv fragment.

In some embodiments, the antibodies or antibody fragments thereof, or antigen binding fragments thereof do not cause a significant level of red blood cell depletion and/or platelet depletion 1 day, 2 days, 4 days, 8 days, 10 days, 15 days, 20 days, 25 days, and/or 30 days after administration.

The term “significant level of cell depletion” may relate to a level of cell depletion which has adverse consequences for the subject.

In some embodiments, the antibodies or antigen binding fragments thereof do not cause a significant level of red blood cell depletion and/or platelet depletion 1 day after administration.

In some embodiments, the antibodies or antigen binding fragments thereof do not cause a significant level of red blood cell depletion and/or platelet depletion 2 days after administration.

In some embodiments, the antibodies or antigen binding fragments thereof do not cause a significant level of red blood cell depletion and/or platelet depletion 4 days after administration.

In some embodiments, the antibodies or antigen binding fragments thereof do not cause a significant level of red blood cell depletion and/or platelet depletion 8 days after administration.

In some embodiments, the antibodies or antigen binding fragments thereof do not cause a significant level of red blood cell depletion and/or platelet depletion 10 days after administration.

In some embodiments, the antibodies or antigen binding fragments thereof do not cause a significant level of red blood cell depletion and/or platelet depletion 15 days after administration.

In some embodiments, the antibodies or antigen binding fragments thereof do not cause a significant level of red blood cell depletion and/or platelet depletion 20 days after administration.

In some embodiments, the antibodies or antigen binding fragments thereof do not cause a significant level of red blood cell depletion and/or platelet depletion 25 days after administration.

In some embodiments, the antibodies or antigen binding fragments thereof do not cause a significant level of red blood cell depletion and/or platelet depletion 30 days after administration.

Suitably, the antibodies or antigen binding fragments thereof for use according to the present invention may result in less than 10%, less than 9%, less than 8%, less than 7%, less than 6%, less than 5%, less than 4%, less than 3%, less than 2%, less than 1% depletion of RBCs after treatment. Suitably, the antibodies or antigen binding fragments thereof for use according to the present invention may result in less than 10%, less than 9%, less than 8%, less than 7%, less than 6%, less than 5%, less than 4%, less than 3%, less than 2%, less than 1% depletion of platelets after treatment.

### Antibody Modifications

The present invention further provides variant anti-CD38 antibodies or antigen binding fragments thereof. That is, there are a number of modifications that can be made to the antibodies or antigen binding fragments thereof of the invention, including, but not limited to, amino acid modifications in the CDRs (affinity maturation), amino acid modifications in the Fc region, glycosylation variants, covalent modifications of other types, etc.

The term “variant” means a polypeptide that differs from that of a parent polypeptide. Amino acid variants can include substitutions, insertions and deletions of amino acids. In general, variants can include any number of modifications, as long as the function of the protein is still present, as described herein. That is, in the case of amino acid variants generated with the CDRs of AB79, for example, the antibody or antigen binding fragment or antibody variant thereof should still specifically bind to both human and cynomolgus CD38. The term “variant Fc region” means an Fc sequence that differs from that of a wild-type or parental Fc sequence by virtue of at least one amino acid modification. Fc variant may refer to the Fc polypeptide itself, compositions comprising the Fc variant polypeptide, or the amino acid sequence. If amino acid variants are generated with the Fc region, for example, the variant antibodies should maintain the required functions for the particular application or indication of the antibody. For example, 1, 2, 3, 4, 5, 6, 7, 8, 9 or 10 amino acid substitutions can be utilized, for example, 1-10, 1-5, 1-4, 1-3, and 1-2 substitutions. Suitable modifications can be made at one or more positions as is generally outlined, for example in US Patent Publication No. 2004013210; and U.S. Pat. Nos. 6,086,875; 6,737,056; 7,317,091; 7,670,600; 8,084,582; 8,188,231; 8,367,805; and 8,937,158, all of which are expressly incorporated by reference in their entirety, and in particular for specific amino acid substitutions that increase binding to Fc receptors.

Suitably, the antibody variant or antigen binding fragment thereof maintains the function of the parent sequence, i.e., the variant or fragment is a functional variant or fragment. Suitably, an antibody variant comprising a variant sequence maintains the function of the parent antibody, i.e., the antibody or antigen binding fragment thereof comprising a variant sequence is able to bind human CD38 and/or cynomolgus CD38. Suitably, treatment with the variant or fragment may result in less than 10%, less than 9%, less than 8%, less than 7%, less than 6%, less than 5%, less than 4%, less than 3%, less than 2%, less than 1% depletion of RBCs. Suitably, treatment with the variant or fragment may result in less than 10%, less than 9%, less than 8%, less than 7%, less than 6%, less than 5%, less than 4%, less than 3%, less than 2%, less than 1% depletion of platelets.

A variant can be considered in terms of similarity (i.e., amino acid residues having similar chemical properties/functions), preferably a variant is expressed in terms of sequence identity.

Sequence comparisons can be conducted by eye, or more usually, with the aid of readily available sequence comparison programs. These publicly and commercially available computer programs can calculate sequence identity between two or more sequences.

It may be desirable to have from 1-5 modifications in the Fc region of wild-type or engineered proteins, as well as from 1 to 5 modifications in the Fv region, for example. A variant polypeptide sequence will preferably possess at least about 80%, 85%, 90%, 91%, 92%, 93%, 94%, 95%, 96%, 97%, 98% or 99% identity to the parent sequences (e.g., the variable regions, the constant regions, and/or the heavy and light chain sequences for AB79). Suitably, the variant may have at least 80% sequence identity to the parent sequence. Suitably, the variant may have at least 85% sequence identity to the parent sequence. Suitably, the variant may have at least 90% sequence identity to the parent sequence. Suitably, the variant may have at least 92% sequence identity to the parent sequence. Suitably, the variant may have at least 95% sequence identity to the parent sequence. Suitably, the variant may have at least 97% sequence identity to the parent sequence. Suitably, the variant may have at least 98% sequence identity to the parent sequence. Suitably, the variant may have at least 99% sequence identity to the parent sequence.

In one embodiment, the sequence identity is determined across the entirety of the sequence. In one embodiment, the sequence identity is determined across the entirety of the candidate sequence being compared to a sequence recited herein.

### Inhibition of CD38 Activity and Side Effect Reduction

The disclosed anti-CD38 antibodies or antigen binding fragments thereof may inhibit cell growth. The term “inhibits growth” refers to any measurable decrease in cell growth when contacted with an anti-CD38 antibody, as compared to the growth of the same cells not in contact with an anti-CD38 antibody, e.g., an inhibition of growth of a cell culture by at least about 10%, 20%, 30%, 40%, 50%, 60%, 70%, 80%, 90%, 99%, or 100%. Suitably, an inhibition of growth may be at least about 70%. Suitably, an inhibition of growth may be at least about 80%. Suitably, an inhibition of growth may be at least about 90%.

In some embodiments, the disclosed anti-CD38 antibodies or antigen binding fragments thereof are able to deplete activated lymphocytes and plasma cells. The term “depletion” in this context means a measurable decrease in serum levels of activated lymphocytes and/or plasma cells in a subject as compared to untreated subjects. In general, depletions of at least about 10%, 20%, 30%, 40%, 50%, 60%, 70%, 80%, 90%, 99%, or 100% are seen. Suitably, the depletion may be at least 50%. Suitably, the depletion may be at least 60%. Suitably, the depletion may be at least 70%. Suitably, the depletion may be at least 80%. Suitably, the depletion may be at least 90%. Suitably depletion may be 100%. As shown below in the Examples, one particular advantage that the antibodies or antigen binding fragments thereof of the present invention exhibit is the recoverability of these cells after dosing; that is, as is known for some treatments (for example with anti-CD20 antibodies for example), cell depletion can last for long periods of time, causing unwanted side effects. As shown herein, the effects on the activated lymphocytes and/or plasma cells are recoverable.

The anti-CD38 antibodies or antigen binding fragments thereof of the present invention allow for reduced side effects compared to prior art anti-CD38 antibodies. In some embodiments, the antibody or antigen binding fragment thereof for use according to the present invention, e.g., AB79 does not induce TEAEs. In some embodiments, the antibody or antigen binding fragment thereof for use according to the present invention, e.g., AB79 allows for a reduction in the incidence of TEAEs in a patient population as compared to other anti-CD38 antibodies, such as MOR202. TEAEs are typically referred to by grades 1, 2, 3, 4, and 5, grade 1 being the least severe and grade 5 being the most severe TEAE. Based on FDA and other guidelines for Common Terminology Criteria for Adverse Events (CTCAE) standards for oncology drugs (see, e.g., https://evs.nci.nih.gov/ftpl/CTCAE/CTCAE_4.03_2010-06-14_QuickReference_5x7.pdf; as well as https://ctep.cancer.gov/protocoldevelopment/electronic_applications/ctc.htm; and Nilsson and Koke (2001) Drug Inform. J. 35: 1289-1299) the following is how such grades are generally determined. Grade 1 is mild: asymptomatic or mild symptoms; clinical or diagnostic observations only; no intervention indicated. Grade 2 is moderate: minimal, local or noninvasive intervention indicated; limiting age-appropriate instrumental activities of daily living (“ADL”). Grade 3 is severe or medically significant but not immediately life-threatening: hospitalization or prolongation of hospitalization indicated; disabling; limiting self-care ADL. Grade 4 is life-threatening consequence: urgent intervention indicated. Grade 5 is death related to AE.

In some embodiments, the antibody or antigen binding fragment thereof for use according to the present invention, e.g., AB79 allows for a reduction in the grade of the TEAEs in a patient population as compared to other anti-CD38 antibodies, such as MOR202. In some embodiments, the antibody or antigen binding fragment thereof for use according to the present invention, e.g., AB79 allows for a reduction in the grade of the TEAEs as compared to other anti-CD38 antibodies from grade 5 to grade 4. In some embodiments, the antibody or antigen binding fragment thereof for use according to the present invention, e.g., AB79 allows for a reduction in the grade of the TEAEs as compared to other anti-CD38 antibodies from grade 4 to grade 3. In some embodiments, the antibody or antigen binding fragment thereof for use according to the present invention, e.g., AB79 allows for a reduction in the grade of the TEAEs as compared to other anti-CD38 antibodies from grade 3 to grade 2. In some embodiments, the antibody or antigen binding fragment thereof for use according to the present invention, e.g., AB79 allows for a reduction in the grade of the TEAEs as compared to other anti-CD38 antibodies from grade 2 to grade 1.

In some embodiments, the antibody or antigen binding fragment thereof for use according to the present invention, e.g., AB79 allows for a reduction in grade of one or more TEAEs selected from the group consisting of anemia (including hemolytic anemia), thrombocytopenia, fatigue, infusion-related reactions (IRRs), leukopenia, lymphopenia, and nausea. In some embodiments, the antibody or antigen binding fragment thereof for use according to the present invention, e.g., AB79 allows for a reduction in the occurrence of one or more TEAEs selected from the group consisting of anemia (including hemolytic anemia), thrombocytopenia, fatigue, infusion-related reactions (IRRs), leukopenia, lymphopenia, and nausea.

In some embodiments, the anti-CD38 antibody or antigen binding fragment thereof results in less than 50%, less than 40%, less than 30%, less than 20%, less than 10%, less than 5%, less than 4%, less than 3%, less than 2%, or less than 1%, depletion of RBCs. In some embodiments, the AB79 antibody results in less than 50%, less than 40%, less than 30%, less than 20%, less than 10%, less than 5%, less than 4%, less than 3%, less than 2%, or less than 1%, depletion of RBCs. In some embodiments, the AB79 antibody or antigen binding fragment thereof results in less than 10% depletion of RBCs.

In some embodiments, the anti-CD38 antibody or antigen binding fragment thereof results in less than 50%, less than 40%, less than 30%, less than 20%, less than 10%, less than 5%, less than 4%, less than 3%, less than 2%, or less than 1%, depletion of platelets. In some embodiments, the AB79 antibody or antigen binding fragment thereof results in less than 50%, less than 40%, less than 30%, less than 20%, less than 10%, less than 5%, less than 4%, less than 3%, less than 2%, or less than 1%, depletion of platelets. In some embodiments, the AB79 antibody or antigen binding fragment thereof results in less than 10% depletion of platelets.

In some embodiments, a diagnostic test is used for determining the presence and/or grade of anemia, including hemolytic anemia. Diagnostic tests for anemia, including hemolytic anemia including measuring the hemoglobin level. Generally, hemoglobin levels are interpreted as follows: (i) very mild/absent anemia: ≥12.0 g/dL, (ii) mild: 10-12 g/dL, (iii) moderate: 8-10 g/dL, (iv) severe: 6-8 g/dL, and (v) very severe: ≤6 g/dL. Other diagnostic tests for anemia, including hemolytic anemia, include measuring the haptoglobin level. Generally, a haptoglobin level ≤25 mg/dL is indicative of the presence of anemia, including hemolytic anemia. Other diagnostic tests include the direct antiglobulin test (DAT) (also referred to as the direct Coombs Test), which is used to determine whether RBCs have been coated in vivo with immunoglobulin, complement, or both.

In some embodiments, a diagnostic test is used for determining the presence and/or grade of thrombocytopenia. Generally, the diagnostic test of thrombocytopenia includes measuring the number of platelets per microliter (μL) blood. Normally, there are 150×10³-450×10³ platelets per μL blood. Generally, thrombocytopenia is diagnosed when there is <150×10³ platelets per μL blood. Mild thrombocytopenia is generally diagnosed if there is 70-150×10³ per μL blood. Moderate thrombocytopenia is generally diagnosed if there is 20-70×10³ per μL. Severe thrombocytopenia is generally diagnosed if there is <20×10³ per μL blood.

### Disease Indications

The antibodies, methods, and dosage units of the invention find use in a variety of applications, including treatment or amelioration of CD38-related diseases. The therapeutic anti-CD38 antibodies or antigen binding fragments thereof of the present invention bind to CD38 positive cells, resulting in depletion of these cells through multiple mechanisms of action, including both CDC and ADCC pathways.

It is known in the art that certain conditions are associated with cells that express CD38, and that certain conditions are associated with the overexpression, high-density expression, or upregulated expression of CD38 on the surfaces of cells. Whether a cell population expresses CD38 or not can be determined by methods known in the art, for example flow cytometric determination of the percentage of cells in a given population that are labeled by an antibody that specifically binds CD38 or immunohistochemical assays, as are generally described below for diagnostic applications. For example, a population of cells in which CD38 expression is detected in about 10-30% of the cells can be regarded as having weak positivity for CD38; and a population of cells in which CD38 expression is detected in greater than about 30% of the cells can be regarded as definite positivity for CD38 (Jackson et al. (1988) Clin. Exp. Immunol. 72: 351-356), though other criteria can be used to determine whether a population of cells expresses CD38. Density of expression on the surfaces of cells can be determined using methods known in the art, such as, for example, flow cytometric measurement of the mean fluorescence intensity of cells that have been fluorescently labeled using antibodies that specifically bind CD38.

In one aspect, the invention provides methods of treating a condition associated with proliferation of cells expressing CD38, comprising administering to a patient a pharmaceutically effective amount of a disclosed antibody or antigen binding fragment thereof in combination with (a) lenolidomide, (b) lenolidomide and bortezomib, or (c) pomolidomide. In some embodiments, the condition is cancer, and in particular embodiments, the cancer is a hematological cancer. In some embodiments, the condition is multiple myeloma, chronic lymphoblastic leukemia, chronic lymphocytic leukemia, plasma cell leukemia, acute myeloid leukemia, chronic myeloid leukemia, B-cell lymphoma, or Burkitt lymphoma. In a particular embodiment, the condition is multiple myeloma.

In some embodiments of the invention, the hematologic cancer is a selected from the group of chronic lymphocytic leukemia, chronic myelogenous leukemia, acute myelogenous leukemia, and acute lymphocytic leukemia. In some embodiments of the invention, the hematologic cancer is chronic lymphocytic leukemia. In some embodiments of the invention, the hematologic cancer is chronic myelogenous leukemia. In some embodiments of the invention, the hematologic cancer is acute myelogenous leukemia. In some embodiments of the invention, the hematologic cancer is acute lymphocytic leukemia.

In some embodiments, the condition is multiple myeloma.

### Multiple Myeloma (MM)

Multiple myeloma (MM) is a malignant disorder of the B cell lineage characterized by neoplastic proliferation of plasma cells in the bone marrow. Pharmacologic findings in healthy volunteers supported further investigation in MM (Fedyk et al. (2018) Blood 132:3249, incorporated herein by reference in its entirety). Proliferation of myeloma cells causes a variety of effects, including lytic lesions (holes) in the bone, decreased red blood cell number, production of abnormal proteins (with attendant damage to the kidney, nerves, and other organs), reduced immune system function, and elevated blood calcium levels (hypercalcemia). Currently treatment options include chemotherapy, preferably associated when possible with autologous stem cell transplantation (ASCT). These treatment regimens exhibit moderate response rates. However, only marginal changes in overall survival are observed and the median survival is approximately 3 years. Thus, there is a critical unmet medical need for the treatment of multiple myeloma. In some embodiments, methods for treating multiple myeloma using the disclosed antibodies or antigen binding fragments thereof are provided.

### Monoclonal Gammopathy Of Undetermined Significance (MGUS) And Smoldering Multiple Myeloma (SMM)

Monoclonal gammopathy of undetermined significance (MGUS) and smoldering multiple myeloma (SMM) are asymptomatic, pre-malignant disorders characterized by monoclonal plasma cell proliferation in the bone marrow and absence of end-organ damage.

Smoldering multiple myeloma (SMM) is an asymptomatic proliferative disorder of plasma cells with a high risk of progression to symptomatic, or active multiple myeloma (Kyle et al. (2007) N. Engl. J. Med. 356(25): 2582-2590). International consensus criteria defining SMM were adopted in 2003 and require that a patient have a M-protein level of >30 g/L and/or bone marrow clonal plasma cells >10% (Internat. Myeloma Working Group (2003) Br. J. Haematol. 121: 749-757). The patients must have no organ or related tissue impairment, such as bone lesions or symptoms. Recent studies have identified two subsets of SMM: i) patients with evolving disease and ii) patients with non-evolving disease (Internat. Myeloma Working Group (2003) Br. J. Haematol. 121: 749-757).

SMM resembles monoclonal gammopathy of undetermined significance (MGUS) as end-organ damage is absent (Kyle et al. (2007) N. Engl. J. Med. 356(25): 2582-2590). Clinically, however, SMM is far more likely to progress to active multiple myeloma or amyloidosis at 20 years (78% probability for SMM vs. 21% for MGUS) (Kyle et al. (2007) N. Engl. J. Med. 356(25): 2582-2590).

International consensus criteria defining MGUS require that a patient have a M-protein level of <30 g/L, bone marrow plasma cells <10% and the absence of organ or related tissue impairment, including bone lesions or symptoms (Internat. Myeloma Working Group (2003) Br. J. Haematol. 121: 749-757).

### Systemic Light Chain Amyloidosis

Amyloidosis refers to a family of protein misfolding diseases in which different types of proteins aggregate as extracellular insoluble fibrils. These are complex, multisystem diseases. A common type of systemic amyloidosis is systemic light chain (AL) amyloidosis. (Gertz et al. (2004) Am. Soc. Hematol. 2004: 257-82). Like multiple myeloma, AL amyloidosis is a plasma cell neoplasm. AL amyloidosis is a rare, progressive, and lethal disease of older adults caused by a small clonal plasma cell population in the bone marrow that produces excess monoclonal immunoglobulin free light chains. Once in circulation, these pathologic light chains misfold, aggregate, and deposit as fibrillar material in visceral organs. The amyloid fibril deposits are the same free light chain protein secreted by the clonal plasma cell. (Cohen and Comenzo (2010) Am. J. Hematol. 2010: 287-94; Merlini and Bellotti (2003) New England J. Med. 349(6): 583-96; Murray et al. (2010) Blood (ASH Annual Meeting Abstracts) 116 (21): abstr 1909). End organ damage and ultimately death is caused as a result of this amyloid fibril deposition. Therapies that suppress the clonal plasma cells ameliorate AL amyloidosis disease by removing the factory producing the circulating toxic free light chains, which then can improve organ function and survival. No treatment has received regulatory approval for systemic AL amyloidosis. Agents used are those used to treat multiple myeloma. Thus, there is a critical unmet medical need for the treatment of patients with AL amyloidosis and targeting CD38 on plasma cells is a relevant therapeutic strategy.

Of particular use in some embodiments are the use of the present antibodies or antigen binding fragments thereof for the use in the diagnosis and/or treatment of a number of diseases, including, but not limited to autoimmune diseases, including but not limited to systemic lupus erythematosus (SLE), rheumatoid arthritis (RA), inflammatory bowel disease (IBD), ulcerative colitis, systemic light chain amyloidosis, and graft-v-host disease. In one aspect, the disease is systemic lupus erythematosus (SLE). In one aspect, the disease is rheumatoid arthritis (RA). In one aspect, the disease is inflammatory bowel disease (IBD). In one aspect, the disease is ulcerative colitis. In one aspect, the disease is graft-v-host disease. In one aspect, the disease is systemic light chain amyloidosis.

Thus, for example, patients with high plasma cell content can be treated, such as SLE patients who exhibit high plasma cell levels, as well as RA patients shown to be unresponsive to CD20 based therapies.

### Antibody Compositions for In Vivo Administration

Formulations of the antibodies or antigen binding fragments thereof used in accordance with the present invention are prepared for storage by mixing an antibody or antigen binding fragment thereof having the desired degree of purity with optional pharmaceutically acceptable carriers, excipients or stabilizers (Remington's Pharmaceutical Sciences 16th edition (1980) Osol, A. Ed.), in the form of lyophilized formulations or aqueous solutions.

The formulations herein may also contain more than one active compound as necessary for the particular indication being treated, preferably those with complementary activities that do not adversely affect each other. For example, it may be desirable to provide antibodies or antigen binding fragments thereof with other specificities. Alternatively, or in addition, the composition may comprise a cytotoxic agent, cytokine, growth inhibitory agent and/or small molecule antagonist. Such molecules are suitably present in combination in amounts that are effective for the purpose intended.

### Subcutaneous Administration

The anti-CD38 antibodies or antigen binding fragments thereof described herein, such as AB79, can be administered at sufficiently dosages that are therapeutically effective, thereby allowing for subcutaneous administration. Subcutaneous administration is a minimally invasive mode of administration and is considered the most versatile and therefore desirable mode of administration that can be used for short term and long term therapies. In some embodiments, subcutaneous administration can be performed by injection. In some embodiments, the site of the injection or device can be rotated when multiple injections or devices are needed.

Accordingly, subcutaneous formulations are much easier for a patient to self-administer, especially since the formulation may have to be taken regularly during the patient's entire life (e.g., starting as early as a child's first year of life). Furthermore, the ease and speed of subcutaneous delivery allows increased patient compliance and quicker access to medication when needed. Thus, the subcutaneous formulations of the anti-CD38 antibodies or antigen binding fragments thereof provided herein provide a substantial benefit over the prior art and solve certain unmet needs.

In some embodiments, the antibodies or antigen binding fragments thereof of the invention are administered to a subject in accordance with known methods via a subcutaneous route. In some embodiments, antibodies or antigen binding fragments thereof of the present invention can be administered by subcutaneous injection. In specific embodiments, the subcutaneous formulation is subcutaneously injected into the same site of a patient (e.g., administered to the upper arm, anterior surface of the thigh, lower portion of the abdomen, or upper back) for repeat or continuous injections. In other embodiments, the subcutaneous formulation is subcutaneously injected into a different or rotating site of a patient. Single or multiple administrations of the formulations may be employed.

In some embodiments, the subcutaneous unit dosage forms described herein can be used for the treatment of cancer. In some embodiments, the subcutaneous unit dosage forms described herein can be used for the treatment of a hematological cancer. In some embodiments, the subcutaneous unit dosage forms described herein can be used for the treatment of multiple myeloma.

In some embodiments, the antibodies or antigen binding fragments thereof of the invention have increased bioavailability as compared to prior art antibodies. In some embodiments, the bioavailability of the antibodies or antigen binding fragments thereof of the present invention is increased 10%, 20%, 30%, 40%, 50%, 60%, 70%, 80%, 90%, or 100% or more as compared to a prior art antibody that binds to human RBCs. In some embodiments, the bioavailability of the antibodies or antigen binding fragments thereof of the present invention that is 110%, 120%, 130%, 140%, 150%, 160%, 170%, 180%, 190%, 200%, 250%, or 300% or more as compared to a prior art antibody that binds to human RBCs. Suitably, the bioavailability may be increased 50%. Suitably, the bioavailability may be increased 60%. Suitably, the bioavailability may be increased 70%. Suitably, the bioavailability may be increased 80%. Suitably, the bioavailability may be increased 90%.

In some embodiments, the increase in bioavailability allows for subcutaneous administration.

In some embodiments, the antibodies or antigen binding fragments thereof of the invention lead to depletion of NK cells, B cells and/or T cells. In some embodiments, the antibodies or antigen binding fragments thereof of the invention allow for increased depletion of NK cells as compared to the depletion of B cells or T cells. In some embodiments, the antibodies or antigen binding fragments thereof of the invention allow for increased depletion of NK cells as compared to B cells, as well as increased depletion of NK cells as compared to T cells. In some embodiments, the antibodies or antigen binding fragments thereof of the invention allow for increased depletion of NK cells as compared to B cells, as well as increased depletion of B cells as compared to T cells. In some embodiments, the antibodies or antigen binding fragments thereof of the invention allow for increased depletion of NK cells as compared to B cells and increased depletion of B cells as compared to T cells. Suitably, the antibodies or antigen binding fragments thereof of the invention may allow for increased depletion of CD38+ cells as compared to CD38⁻ cells.

In certain embodiments, the bioavailability of the anti-CD38 antibodies or antigen binding fragments thereof described herein after subcutaneous administration is between at least 50% and at least 80% as compared to intravenous administration normalized for the same dose. In certain embodiments, the bioavailability of the anti-CD38 antibodies or antigen binding fragments thereof described herein after subcutaneous administration is between at least 60% and at least 80% as compared to intravenous administration normalized for the same dose. In certain embodiments, the bioavailability of the anti-CD38 antibodies or antigen binding fragments thereof described herein after subcutaneous administration is between at least 50% and 70% as compared to intravenous administration normalized for the same dose. In certain embodiments, the bioavailability of the anti-CD38 antibodies or antigen binding fragments thereof described herein after subcutaneous administration is between at least 55% and 65% as compared to intravenous administration normalized for the same dose. In certain embodiments, the bioavailability of the anti-CD38 antibodies or antigen binding fragments thereof described herein after subcutaneous administration is between at least 55% and 70% as compared to intravenous administration normalized for the same dose.

In certain embodiments, the bioavailability of the anti-CD38 antibodies or antigen binding fragments thereof described herein after subcutaneous administration is at least 40%, at least 45%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, or at least 85% as compared to intravenous administration normalized for the same dose. Suitably the bioavailability may be at least 50% as compared to intravenous administration normalized for the same dose. Suitably the bioavailability may be at least 60% as compared to intravenous administration normalized for the same dose. Suitably the bioavailability may be at least 70% as compared to intravenous administration normalized for the same dose. Suitably the bioavailability may be at least 80% as compared to intravenous administration normalized for the same dose. Suitably the bioavailability may be at least 90% as compared to intravenous administration normalized for the same dose.

In some embodiments, the present disclosure provides a method wherein the bioavailability of the antibodies or antigen binding fragments thereof of the invention after subcutaneous administration is 50%-80% as compared to intravenous administration normalized for the same dose.

In some embodiments, the present disclosure provides a method wherein the bioavailability of the antibodies or antigen binding fragments thereof of the invention after subcutaneous administration is at least 50% as compared to intravenous administration normalized for the same dose.

In some embodiments, the present disclosure provides a method wherein the bioavailability of the antibodies or antigen binding fragments thereof of the invention after subcutaneous administration is at least 55% as compared to intravenous administration normalized for the same dose.

In some embodiments, the present disclosure provides a method wherein the bioavailability of the antibodies or antigen binding fragments thereof of the invention after subcutaneous administration is at least 60% as compared to intravenous administration normalized for the same dose.

In some embodiments, the present disclosure provides a method wherein the bioavailability of the antibodies or antigen binding fragments thereof of the invention after subcutaneous administration is at least 65% as compared to intravenous administration normalized for the same dose.

In some embodiments, the present disclosure provides a method wherein the bioavailability of the antibodies or antigen binding fragments thereof of the invention after subcutaneous administration is at least 70% as compared to intravenous administration normalized for the same dose.

In some embodiments, the present disclosure provides a method wherein the bioavailability of the antibodies or antigen binding fragments thereof of the invention after subcutaneous administration is at least 75% as compared to intravenous administration normalized for the same dose.

In some embodiments, the present disclosure provides a method wherein the bioavailability of the antibodies or antigen binding fragments thereof of the invention after subcutaneous administration is at least 80% as compared to intravenous administration normalized for the same dose.

In some embodiments, the present disclosure provides the unit dosage form comprising the anti-CD38 antibody or antigen binding fragment thereof as described herein, wherein the anti-CD38 antibody results in less than 10% depletion of RBCs.

In some embodiments, the present disclosure provides the unit dosage form comprising the anti-CD38 antibody or antigen binding fragment thereof as described herein, wherein the anti-CD38 antibody results in less than 10% depletion of platelets.

In certain embodiments, the anti-CD38 antibodies or antigen binding fragments thereof described herein are subcutaneously administered in a single bolus injection. In certain embodiments, the anti-CD38 antibodies or antigen binding fragments thereof described herein are subcutaneously administered monthly. In certain embodiments, the anti-CD38 antibodies or antigen binding fragments thereof described herein are subcutaneously administered every two weeks. In certain embodiments, the anti-CD38 antibodies or antigen binding fragments thereof described herein are subcutaneously administered weekly. In certain embodiments, the anti-CD38 antibodies or antigen binding fragments thereof described herein are subcutaneously administered twice a week. In certain embodiments, the anti-CD38 antibodies or antigen binding fragments thereof described herein are subcutaneously administered daily. In certain embodiments, the anti-CD38 antibodies or antigen binding fragments thereof described herein are subcutaneously administered every 12 hours. In certain embodiments, the anti-CD38 antibodies or antigen binding fragments thereof described herein are subcutaneously administered every 8 hours. In certain embodiments, the anti-CD38 antibodies or antigen binding fragments thereof described herein are subcutaneously administered every six hours. In certain embodiments, the anti-CD38 antibodies or antigen binding fragments thereof described herein are subcutaneously administered every four hours. In certain embodiments, the anti-CD38 antibodies or antigen binding fragments thereof described herein are subcutaneously administered every two hours. In certain embodiments, the anti-CD38 antibodies or antigen binding fragments thereof described herein are subcutaneously administered every hour.

In some embodiments, the therapeutic anti-CD38 antibodies or antigen binding fragments thereof are formulated as part of a unit dosage form. In some embodiments, the anti-CD38 antibody or antigen binding fragment thereof comprises a heavy chain comprising the following CDR amino acid sequences: GFTFDDYG (SEQ ID NO:3; HCDR1 AB79), ISWNGGKT (SEQ ID NO:4; HCDR2 AB79), and ARGSLFHDSSGFYFGH (SEQ ID NO:5; HCDR3 AB79) or variants of those sequences having up to three amino acid changes. In some embodiments, the antibody or antigen binding fragment thereof comprises a light chain comprising the following CDR amino acid sequences: SSNIGDNY (SEQ ID NO:6; LCDR1 AB79), RDS (SEQ ID NO:7; LCDR2 AB79), and QSYDSSLSGS (SEQ ID NO:8; LCDR3 AB79) or variants of those sequences having up to three amino acid changes. In some embodiments, the antibody or antigen binding fragment thereof comprises a heavy chain comprising the following CDR amino acid sequences: GFTFDDYG (SEQ ID NO:3; HCDR1 AB79), ISWNGGKT (SEQ ID NO:4; HCDR2 AB79), ARGSLFHDSSGFYFGH (SEQ ID NO:5; HCDR3 AB79) or variants of those sequences having up to three amino acid changes and a light chain comprising the following CDR amino acid sequences: SSNIGDNY (SEQ ID NO:6; LCDR1 AB79), RDS (SEQ ID NO:7; LCDR2 AB79), and QSYDSSLSGS (SEQ ID NO:8; LCDR3 AB79) or variants of those sequences having up to three amino acid changes. In some embodiments, the antibody or antigen binding fragment thereof comprises a heavy chain comprising the following CDR amino acid sequences: GFTFDDYG (SEQ ID NO:3; HCDR1 AB79), ISWNGGKT (SEQ ID NO:4; HCDR2 AB79), and ARGSLFHDSSGFYFGH (SEQ ID NO:5; HCDR3 AB79). In some embodiments, the antibody or antigen binding fragment thereof comprises a light chain comprising the following CDR amino acid sequences: SSNIGDNY (SEQ ID NO:6; LCDR1 AB79), RDS (SEQ ID NO:7; LCDR2 AB79), and QSYDSSLSGS (SEQ ID NO:8; LCDR3 AB79). In some embodiments, the antibody or antigen binding fragment thereof comprises a heavy chain comprising the following CDR amino acid sequences: GFTFDDYG (SEQ ID NO:3; HCDR1 AB79), ISWNGGKT (SEQ ID NO:4; HCDR2 AB79), ARGSLFHDSSGFYFGH (SEQ ID NO:5; HCDR3 AB79) and a light chain comprising the following CDR amino acid sequences: SSNIGDNY (SEQ ID NO:6; LCDR1 AB79), RDS (SEQ ID NO:7; LCDR2 AB79), and QSYDSSLSGS (SEQ ID NO:8; LCDR3 AB79). In some embodiments, the antibody or antigen binding fragment thereof comprises a heavy chain comprising an amino acid sequence having at least 80% sequence identity to SEQ ID NO:9. Suitably, the heavy chain may comprise the following CDR amino acid sequences: GFTFDDYG (SEQ ID NO:3; HCDR1 AB79), ISWNGGKT (SEQ ID NO:4; HCDR2 AB79), and ARGSLFHDSSGFYFGH (SEQ ID NO:5; HCDR3 AB79) and the remainder of the heavy chain may have at least 80% sequence identity to SEQ ID NO 9. In some embodiments, the antibody or antigen binding fragment thereof comprises a heavy chain comprising the variable heavy (VH) chain amino acid sequence of SEQ ID NO:9.

(SEQ ID NO: 9)
EVQLLESGGGLVQPGGSLRLSCAASGFTFDDYGMSWVRQAPGKGLEWVSDI
SWNGGKTHYVDSVKGQFTISRDNSKNTLYLQMNSLRAEDTAVYYCARGSLF
HDSSGFYFGHWGQGTLVTVSSASTKGPSVFPLA.

In some embodiments, the antibody or antigen binding fragment thereof comprises a light chain comprising an amino acid sequence having at least 80% sequence identity to SEQ ID NO:10. Suitably, the light chain may comprise the following CDR sequences: SSNIGDNY (SEQ ID NO:6; LCDR1 AB79), RDS (SEQ ID NO:7; LCDR2 AB79), and QSYDSSLSGS (SEQ ID NO:8; LCDR3 AB79) and the remainder of the light chain may have at least 80% sequence identity to SEQ ID NO: 10. In some embodiments, the antibody or antigen binding fragment thereof comprises a light chain comprising the variable light (VL) chain amino acid sequence of SEQ ID NO:10.

(SEQ ID NO: 10)
QSVLTQPPSASGTPGQRVTISCSGSSSNIGDNYVSWYQQLPGTAPKLLIYR
DSQRPSGVPDRFSGSKSGTSASLAISGLRSEDEADYYCQSYDSSLSGSVFG
GGTKLTVLGQPKANPTVTLFPPSSEEL.

In some embodiments, the antibody or antigen binding fragment thereof comprises a heavy chain comprising the VH region amino acid sequence of SEQ ID NO:9 or a variant thereof as described herein and a light chain comprising the VL region amino acid sequence of SEQ ID NO:10 or a variant thereof as described herein.

As will be appreciated by those in the art, the variable heavy and light chains can be joined to human IgG constant domain sequences, generally IgG1, IgG2 or IgG4.

In some embodiments, the antibody or antigen binding fragment thereof comprises a heavy chain (HC) having amino acid sequence with at least 80% sequence identity to SEQ ID NO:11. Suitably, the heavy chain may comprise the CDR sequences as defined by SEQ ID NO: 3, SEQ ID NO: 4 and SEQ ID NO: 5 and the remainder of the heavy chain may have at least 80% sequence identity to SEQ ID NO 11. In some embodiments, the antibody or antigen binding fragment thereof comprises the heavy chain (HC) amino acid sequence of SEQ ID NO:11.

(SEQ ID NO: 11)
EVQLLESGGGLVQPGGSLRLSCAASGFTFDDYGMSWVRQAPGKGLEWVSDI
SWNGGKTHYVDSVKGQFTISRDNSKNTLYLQMNSLRAEDTAVYYCARGSLF
HDSSGFYFGHWGQGTLVTVSSASTKGPSVFPLAPSSKSTSGGTAALGCLVK
DYFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLEPPKPKD
TLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPGK.

In some embodiments, the antibody or antigen binding fragment thereof comprises a light chain (LC) having amino acid sequence with at least 80% sequence identity to SEQ ID NO:12. Suitably, the light chain may comprise the CDR sequences as defined by SEQ ID NO: 6, SEQ ID NO: 7 and SEQ ID NO: 8 and the remainder of the light chain may have at least 80% sequence identity to SEQ ID NO 12. In some embodiments, the antibody or antigen binding fragment thereof comprises the light chain (LC) amino acid sequence of SEQ ID NO:12.

(SEQ ID NO: 12)
QSVLTQPPSASGTPGQRVTISCSGSSSNIGDNYVSWYQQLPGTAPKLLIYR
DSQRPSGVPDRFSGSKSGTSASLAISGLRSEDEADYYCQSYDSSLSGSVFG
GGTKLTVLGQPKANPTVTLFPPSSEELQANKATLVCLISDFYPGAVTVAWK
ADGSPVKAGVETTKPSKQSNNKYAASSYLSLTPEQWKSHRSYSCQVTHEGS
TVEKTVAPTECS.

In some embodiments, the antibody or antigen binding fragment thereof comprises the HC amino acid sequence of SEQ ID NO:11 or a variant thereof as described herein and the LC amino acid sequence of SEQ ID NO:12 or a variant thereof as described herein.

In some embodiments, the formulation comprising the anti-CD38 antibody or antigen binding fragment thereof is a unit dosage form. In some embodiments, the unit dosage form comprises an amount sufficient to administer a dosage of about 45 mgs to about 1,800 mgs. In some embodiments, the unit dosage form comprises an amount sufficient to administer a dosage of about 45 mgs to about 300 mgs. In some embodiments, the unit dosage form comprises an amount sufficient to administer a dosage of about 135 mgs to about 1,800 mgs. In some embodiments, the unit dosage form comprises an amount sufficient to administer a dosage of about 135 mgs to about 300 mgs. In some embodiments, the unit dosage form comprises an amount sufficient to administer a dosage of about 600 mgs to about 1,800 mgs. In some embodiments, the unit dosage form comprises an amount sufficient to administer a dosage of about 1,200 mgs to about 1,800 mgs. In some embodiments, the unit dosage form comprises an amount sufficient to administer a dosage of about 45 mgs to about 1,200 mgs. In some embodiments, the unit dosage form comprises an amount sufficient to administer a dosage of about 135 mgs to about 1,200 mgs. In some embodiments, the unit dosage form comprises an amount sufficient to administer a dosage of about 300 mgs to about 600 mgs. In some embodiments, the unit dosage form comprises an amount sufficient to administer a dosage of about 600 mgs to about 1,200 mgs. In some embodiments, the unit dosage form comprises an amount sufficient to administer a dosage of about 600 mgs to about 1,200 mgs. In some embodiments, the unit dosage form comprises an amount sufficient to administer a dosage of about 45 mgs to about 135 mgs. In some embodiments, the unit dosage form comprises an amount sufficient to administer a dosage of about 45 mgs to about 600 mgs. In some embodiments, the unit dosage form comprises an amount sufficient to administer a dosage of about 135 mgs to about 600 mgs. In some embodiments, the dosage is in mgs per kilogram bodyweight. In some embodiments, the dosage is a daily dosage. In some embodiments, the unit dosage form comprises an amount sufficient to administer a dosage of about 300 mgs. In some embodiments, the unit dosage form comprises an amount sufficient to administer a dosage of about 600 mgs.

In some embodiments, the anti-CD38 antibody or antigen binding fragment thereof unit dosage forms provided herein may further comprise one or more pharmaceutically acceptable excipients, carriers, and/or diluents. In some embodiments, the anti-CD38 antibody or antigen binding fragment thereof is provided as a pharmaceutical composition which comprises a unit dosage form according to the present invention. Suitably, the pharmaceutical composition may further comprise one or more pharmaceutically acceptable excipients, carriers, and/or diluents.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Compositions may be formulated in dosage unit form for ease of administration and uniformity of dosage. Dosage unit forms as used herein can, in some embodiments, refer to physically discrete units suited as unitary dosages for the subjects to be treated, each unit containing a predetermined quantity of active compound calculated to produce the desired therapeutic effect in association with the required pharmaceutical carrier.

The specification for the dosage unit forms of the present invention are dictated by and are directly dependent on (a) the unique characteristics of the active compound and the particular therapeutic effect to be achieved, and (b) the limitations inherent in the art of compounding such an active compound for the treatment of an individual.

The efficient dosages and the dosage regimens for the anti-CD38 antibodies or antigen binding fragments thereof used in the present invention depend on the type and severity of the disease or condition to be treated and may be determined by persons skilled in the art.

In one embodiment, the therapeutic antibody or antigen binding fragment thereof is formulated at 100 mg/ml concentration. In some embodiments, 1.75 mL, 2.0 mL, 2.25 mL or 2.5 mL volume is injected in the thigh, abdomen, or arm. In some embodiments, 1.75 mL, 2.0 mL, 2.25 mL or 2.5 mL volume is injected in the thigh or abdomen. In some embodiments, 2.25 mL volume is injected in the thigh or abdomen. In some embodiments, the dose is administered over a 4-, 6-, 8-, or 10-hour period of time. In some embodiments, the dose is administered over an 8-hour period of time. In some embodiments, 2, 4, 6, or 8 doses are administered. In some embodiments, 2 doses are administered. In some embodiments, 4 doses are administered. In some embodiments, 6 doses are administered. In some embodiments, 8 doses are administered. In some embodiments, the doses are administered every 2 hours.

In a further embodiment, the anti-CD38 antibody or antigen binding fragment thereof is administered once weekly for 2 to 12 weeks. Suitably, the antibody or antigen binding fragment thereof may be administered once weekly, such as for 3 to 10 weeks. Suitably, the antibody or antigen binding fragment thereof may be administered once weekly, such as for 4 to 8 weeks. Suitably, the antibody or antigen binding fragment thereof may be administered once weekly, such as for 5 to 7 weeks.

In an embodiment, the anti-CD38 antibody or antigen binding fragment thereof is administered subcutaneously at a frequency that changes over time. Suitably, the antibody or antigen binding fragment thereof may be administered, once weekly for 8 weeks, then once every 2 weeks for 16 weeks, and then once every 4 weeks thereafter in a 28-day treatment cycle until unacceptable toxicities are observed or withdrawal of the subject due to other reasons.

In one embodiment, the anti-CD38 antibody or antigen binding fragment thereof is administered by maintenance therapy, such as, e.g., once a week for a period of 6 months or more.

In one embodiment, the present disclosure provides the unit dosage form comprising the anti-CD38 antibody or antigen binding fragment thereof as described herein, wherein the anti-CD38 antibody results in less than 10% depletion of RBCs.

In one embodiment, the present disclosure provides the unit dosage form comprising the anti-CD38 antibody or antigen binding fragment thereof, (a) lenolidomide, (b) lenolidomide and bortezomib, or (c) pomolidomide as described herein, wherein the anti-CD38 antibody or antigen binding fragment thereof in combination with (a) lenolidomide, (b) lenolidomide and bortezomib, or (c) pomolidomide results in less than 10% depletion of platelets.

In an embodiment, the anti-CD38 antibody or antigen binding fragment thereof of the invention is administered over the course of 1-8 treatment cycles of 28 days in combination with lenolidomide and dexamethasone. In an embodiment, the anti-CD38 antibody or antigen binding fragment thereof of the invention is administered over the course of 1 treatment cycle of 28 days in combination with lenolidomide and dexamethasone. In an embodiment, the anti-CD38 antibody or antigen binding fragment thereof of the invention is administered over the course of 2 treatment cycles of 28 days in combination with lenolidomide and dexamethasone. In an embodiment, the anti-CD38 antibody or antigen binding fragment thereof of the invention is administered over the course of 3 treatment cycles of 28 days in combination with lenolidomide and dexamethasone. In an embodiment, the anti-CD38 antibody or antigen binding fragment thereof of the invention is administered over the course of 4 treatment cycles of 28 days in combination with lenolidomide and dexamethasone. In an embodiment, the anti-CD38 antibody or antigen binding fragment thereof of the invention is administered over the course of 5 treatment cycles of 28 days in combination with lenolidomide and dexamethasone. In an embodiment, the anti-CD38 antibody or antigen binding fragment thereof of the invention is administered over the course of 6 treatment cycles of 28 days in combination with lenolidomide and dexamethasone. In an embodiment, the anti-CD38 antibody or antigen binding fragment thereof of the invention is administered over the course of 7 treatment cycles of 28 days in combination with lenolidomide and dexamethasone. In an embodiment, the anti-CD38 antibody or antigen binding fragment thereof of the invention is administered over the course of 8 treatment cycles of 28 days in combination with lenolidomide and dexamethasone.

In an embodiment, the anti-CD38 antibody or antigen binding fragment thereof is administered on days 1, 8, 15 and 22 of the first two treatment cycles, on days 1 and 15 of the subsequent four treatment cycles and on day 1 of any additional treatment cycles; b) lenolidomide is administered on days 1 to 21 of each treatment cycle; and c) dexamethasone is administered on days 1, 8, 15 and 22 of each 1-8 treatment cycles. In one embodiment, dexamethasone is administered on days 1, 8, 15 and 22 of 1 treatment cycle. In one embodiment, dexamethasone is administered on days 1, 8, 15 and 22 of each of 2 treatment cycles. In one embodiment, dexamethasone is administered on days 1, 8, 15 and 22 of each of 3 treatment cycles. In one embodiment, dexamethasone is administered on days 1, 8, 15 and 22 of each of 4 treatment cycles. In one embodiment, dexamethasone is administered on days 1, 8, 15 and 22 of each of 5 treatment cycles. In one embodiment, dexamethasone is administered on days 1, 8, 15 and 22 of each of 6 treatment cycles. In one embodiment, dexamethasone is administered on days 1, 8, 15 and 22 of each of 7 treatment cycles. In one embodiment, dexamethasone is administered on days 1, 8, 15 and 22 of each of 8 treatment cycles.

In an embodiment, the anti-CD38 antibody or antigen binding fragment thereof of the invention is administered over the course of 1-8 treatment cycles of 28 days in combination with lenolidomide, dexamethasone, and bortezomib. In an embodiment, the anti-CD38 antibody or antigen binding fragment thereof of the invention is administered over the course of 1 treatment cycle of 28 days in combination with lenolidomide, dexamethasone, and bortezomib. In an embodiment, the anti-CD38 antibody or antigen binding fragment thereof of the invention is administered over the course of 2 treatment cycles of 28 days in combination with lenolidomide, dexamethasone, and bortezomib. In an embodiment, the anti-CD38 antibody or antigen binding fragment thereof of the invention is administered over the course of 3 treatment cycles of 28 days in combination with lenolidomide, dexamethasone, and bortezomib. In an embodiment, the anti-CD38 antibody or antigen binding fragment thereof of the invention is administered over the course of 4 treatment cycles of 28 days in combination with lenolidomide, dexamethasone, and bortezomib. In an embodiment, the anti-CD38 antibody or antigen binding fragment thereof of the invention is administered over the course of 5 treatment cycles of 28 days in combination with lenolidomide, dexamethasone, and bortezomib. In an embodiment, the anti-CD38 antibody or antigen binding fragment thereof of the invention is administered over the course of 6 treatment cycles of 28 days in combination with lenolidomide, dexamethasone, and bortezomib. In an embodiment, the anti-CD38 antibody or antigen binding fragment thereof of the invention is administered over the course of 7 treatment cycles of 28 days in combination with lenolidomide, dexamethasone, and bortezomib. In an embodiment, the anti-CD38 antibody or antigen binding fragment thereof of the invention is administered over the course of 8 treatment cycles of 28 days in combination with lenolidomide, dexamethasone, and bortezomib.

In an embodiment, the anti-CD38 antibody or antigen binding fragment thereof is administered on days 1, 8, 15 and 22 of the first two treatment cycles, on days 1 and 15 of the subsequent four treatment cycles and on day 1 of any additional treatment cycles; b) lenolidomide is administered on days 1 to 21 of each treatment cycle; c) dexamethasone is administered on days 1, 8, 15 and 22 of each 1-8 treatment cycles; and d) bortezomib is administered on days 1, 8 and 15 of each 1-8 treatment cycles. In one embodiment, dexamethasone is administered on days 1, 8, 15 and 22 of 1, 2, 3, 4, 5, 6, 7, or 8 treatment cycles, wherein the treatment cycle is 28 days. In one embodiment, dexamethasone is administered on days 1, 8, 15 and 22 of 1 treatment cycle, wherein the treatment cycle is 28 days. In one embodiment, dexamethasone is administered on days 1, 8, 15 and 22 of each of 2 treatment cycles, wherein the treatment cycle is 28 days. In one embodiment, dexamethasone is administered on days 1, 8, 15 and 22 of each of 3 treatment cycles, wherein the treatment cycle is 28 days. In one embodiment, dexamethasone is administered on days 1, 8, 15 and 22 of each of 4 treatment cycles, wherein the treatment cycle is 28 days. In one embodiment, dexamethasone is administered on days 1, 8, 15 and 22 of each of 5 treatment cycles, wherein the treatment cycle is 28 days. In one embodiment, dexamethasone is administered on days 1, 8, 15 and 22 of each of 6 treatment cycles, wherein the treatment cycle is 28 days. In one embodiment, dexamethasone is administered on days 1, 8, 15 and 22 of each of 7 treatment cycles, wherein the treatment cycle is 28 days. In one embodiment, dexamethasone is administered on days 1, 8, 15 and 22 of each of 8 treatment cycles, wherein the treatment cycle is 28 days. In one embodiment, bortezomib is administered on days 1, 8, and 15 of 1, 2, 3, 4, 5, 6, 7, or 8 treatment cycles, wherein the treatment cycle is 28 days. In one embodiment, bortezomib is administered on days 1, 8, and 15 of 1 treatment cycle, wherein the treatment cycle is 28 days. In one embodiment, bortezomib is administered on days 1, 8, and 15 of each of 2 treatment cycles, wherein the treatment cycle is 28 days. In one embodiment, bortezomib is administered on days 1, 8, and 15 of each of 3 treatment cycles, wherein the treatment cycle is 28 days. In one embodiment, bortezomib is administered on days 1, 8, and 15 of each of 4 treatment cycles, wherein the treatment cycle is 28 days. In one embodiment, bortezomib is administered on days 1, 8, and 15 of each of 5 treatment cycles, wherein the treatment cycle is 28 days. In one embodiment, bortezomib is administered on days 1, 8, and 15 of each of 6 treatment cycles, wherein the treatment cycle is 28 days. In one embodiment, bortezomib is administered on days 1, 8, and 15 of each of 7 treatment cycles, wherein the treatment cycle is 28 days. In one embodiment, bortezomib is administered on days 1, 8, and 15 of each of 8 treatment cycles, wherein the treatment cycle is 28 days. In one embodiment, c) dexamethasone is administered on days 1, 8, 15 and 22 of 1 treatment cycle; and d) bortezomib is administered on days 1, 8 and 15 of 1 treatment cycles, wherein the treatment cycle is 28 days. In one embodiment, c) dexamethasone is administered on days 1, 8, 15 and 22 of 2 treatment cycles; and d) bortezomib is administered on days 1, 8 and 15 of 2 treatment cycles, wherein the treatment cycle is 28 days. In one embodiment, c) dexamethasone is administered on days 1, 8, 15 and 22 of 3 treatment cycles; and d) bortezomib is administered on days 1, 8 and 15 of 3 treatment cycles, wherein the treatment cycle is 28 days. In one embodiment, c) dexamethasone is administered on days 1, 8, 15 and 22 of 4 treatment cycles; and d) bortezomib is administered on days 1, 8 and 15 of 4 treatment cycles, wherein the treatment cycle is 28 days. In one embodiment, c) dexamethasone is administered on days 1, 8, 15 and 22 of 5 treatment cycles; and d) bortezomib is administered on days 1, 8 and 15 of 5 treatment cycles, wherein the treatment cycle is 28 days. In one embodiment, c) dexamethasone is administered on days 1, 8, 15 and 22 of 6 treatment cycles; and d) bortezomib is administered on days 1, 8 and 15 of 6 treatment cycles, wherein the treatment cycle is 28 days. In one embodiment, c) dexamethasone is administered on days 1, 8, 15 and 22 of 7 treatment cycles; and d) bortezomib is administered on days 1, 8 and 15 of 7 treatment cycles, wherein the treatment cycle is 28 days. In one embodiment, c) dexamethasone is administered on days 1, 8, 15 and 22 of 8 treatment cycles; and d) bortezomib is administered on days 1, 8 and 15 of 8 treatment cycles, wherein the treatment cycle is 28 days.

In an embodiment, the anti-CD38 antibody or antigen binding fragment thereof of the invention is administered over the course of 1-8 treatment cycles of 28 days in combination with pomolidomide and dexamethasone. In an embodiment, the anti-CD38 antibody or antigen binding fragment thereof of the invention is administered over the course of 1 treatment cycle of 28 days in combination with pomolidomide and dexamethasone. In an embodiment, the anti-CD38 antibody or antigen binding fragment thereof of the invention is administered over the course of 2 treatment cycles of 28 days in combination with pomolidomide and dexamethasone. In an embodiment, the anti-CD38 antibody or antigen binding fragment thereof of the invention is administered over the course of 3 treatment cycles of 28 days in combination with pomolidomide and dexamethasone. In an embodiment, the anti-CD38 antibody or antigen binding fragment thereof of the invention is administered over the course of 4 treatment cycles of 28 days in combination with pomolidomide and dexamethasone. In an embodiment, the anti-CD38 antibody or antigen binding fragment thereof of the invention is administered over the course of 5 treatment cycles of 28 days in combination with pomolidomide and dexamethasone. In an embodiment, the anti-CD38 antibody or antigen binding fragment thereof of the invention is administered over the course of 6 treatment cycles of 28 days in combination with pomolidomide and dexamethasone. In an embodiment, the anti-CD38 antibody or antigen binding fragment thereof of the invention is administered over the course of 7 treatment cycles of 28 days in combination with pomolidomide and dexamethasone. In an embodiment, the anti-CD38 antibody or antigen binding fragment thereof of the invention is administered over the course of 8 treatment cycles of 28 days in combination with pomolidomide and dexamethasone.

In an embodiment, the anti-CD38 antibody or antigen binding fragment thereof is administered on days 1, 8, 15 and 22 of the first two treatment cycles, on days 1 and 15 of the subsequent four treatment cycles and on day 1 of any additional treatment cycles; b) pomolidomide is administered on days 1 to 21 of each treatment cycle; and c) dexamethasone is administered on days 1, 8, 15 and 22 of each 1-8 treatment cycles. In one embodiment, dexamethasone is administered on days 1, 8, 15 and 22 of one treatment cycles. In one embodiment, dexamethasone is administered on days 1, 8, 15 and 22 of each of 2 treatment cycles. In one embodiment, dexamethasone is administered on days 1, 8, 15 and 22 of each of 3 treatment cycles. In one embodiment, dexamethasone is administered on days 1, 8, 15 and 22 of each of 4 treatment cycles. In one embodiment, dexamethasone is administered on days 1, 8, 15 and 22 of each of 5 treatment cycles. In one embodiment, dexamethasone is administered on days 1, 8, 15 and 22 of each of 6 treatment cycles. In one embodiment, dexamethasone is administered on days 1, 8, 15 and 22 of each of 7 treatment cycles. In one embodiment, dexamethasone is administered on days 1, 8, 15 and 22 of each of 8 treatment cycles.

### Treatment Modalities

In the methods of the invention, therapy is used to provide a positive therapeutic response with respect to a disease or condition. The term “positive therapeutic response” refers to an improvement in a disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease. For B cell tumors, the subject may experience a decrease in the so-called B symptoms, e.g., night sweats, fever, weight loss, and/or urticaria. For pre-malignant conditions, therapy with an anti-CD38 therapeutic antibody may block and/or prolong the time before development of a related malignant condition, for example, development of multiple myeloma in subjects suffering from monoclonal gammopathy of undetermined significance (MGUS).

An improvement in the disease may be characterized as a complete response. The term “complete response” refers to the absence of clinically detectable disease with normalization of any previously abnormal radiographic studies, bone marrow, and cerebrospinal fluid (CSF) or abnormal monoclonal protein in the case of myeloma.

Such a response may persist for at least 4 to 8 weeks, or at least 6 to 8 weeks, following treatment according to the methods of the invention. Alternatively, an improvement in the disease may be categorized as being a partial response. The term “partial response” may refer to at least about a 50% decrease in all measurable tumor burden (i.e., the number of malignant cells present in the subject, or the measured bulk of tumor masses or the quantity of abnormal monoclonal protein) in the absence of new lesions, which may persist for 4 to 8 weeks, or 6 to 8 weeks.

Treatment according to the present invention includes a “therapeutically effective amount” of the medicaments used.

The terms “therapeutically effective amount” and “therapeutically effective dosage” refer to an amount of a therapy that is sufficient to reduce or ameliorate the severity and/or duration of a disorder or one or more symptoms thereof; prevent the advancement of a disorder; cause regression of a disorder; prevent the recurrence, development, onset, or progression of one or more symptoms associated with a disorder; or enhance or improve the prophylactic or therapeutic effect(s) of another therapy (e.g., prophylactic or therapeutic agent), at dosages and for periods of time necessary to achieve a desired therapeutic result. A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects. A “therapeutically effective amount” of an antibody for tumor therapy may be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

### Anti-CD38 Antibody Kits

In another aspect of the invention, kits are provided for the treatment of a disease or condition associated with hematological cancers. In one embodiment, the kit comprises a dose of an anti-CD38 antibody or antigen binding fragment thereof described herein, such as AB79 in combination with (a) lenolidomide, (b) lenolidomide and bortezomib, or (c) pomolidomide. In some embodiments, the kits provided herein may contain one or more dose of a liquid or lyophilized formulation as provided herein. When the kits comprise a lyophilized formulation of an anti-CD38 antibody or antigen binding fragment thereof described herein such as AB79, generally the kits will also contain a suitable liquid for reconstitution of the liquid formulation, for example, sterile water or a pharmaceutically acceptable buffer. In some embodiments, the kits may comprise an anti-CD38 antibody or antigen binding fragment thereof formulation described herein prepackaged in a syringe for subcutaneous administration by a health care professional or for home use. In some embodiments, the kits may comprise lenolidomide and dexamethasone for oral, intravenous, or subcutaneous administration in a suitable dosage form. In some embodiments, the kits may comprise lenolidomide, dexamethasone, and bortezomib for oral, intravenous, or subcutaneous administration in a suitable dosage form. In an embodiment, lenolidomide is in an oral dosage form. In certain embodiments, dexamethasone is in oral or iv dosage forms. In an embodiment, bortezomib is in a subcutaneous dosage form.

In certain embodiments, the kit will be for a single administration or dose of an anti-CD38 antibody or antigen binding fragment thereof described herein such as AB79 in combination with (a) lenolidomide, (b) lenolidomide and bortezomib, or (c) pomolidomide. In other embodiments, the kit may contain multiple doses of an anti-CD38 antibody or antigen binding fragment thereof described herein such as AB79 for subcutaneous administration. In one embodiment, the kit may comprise an anti-CD38 antibody or antigen binding fragment thereof formulation described herein prepackaged in a syringe for subcutaneous administration by a health care professional or for home use.

In certain embodiments, the kit will be for a single administration or dose of an anti-CD38 antibody or antigen binding fragment thereof described herein such as AB79 together with lenolidomide and dexamethasone. In other embodiments, the kit may contain multiple doses of an anti-CD38 antibody or antigen binding fragment thereof described herein such as AB79 for subcutaneous administration as well lenolidomide for oral administration and dexamethasone for oral or iv administration. In one embodiment, the kit may comprise an anti-CD38 antibody or antigen binding fragment thereof formulation described herein prepackaged in a syringe for subcutaneous administration by a health care professional or for home use.

In certain embodiments, the kit will be for a single administration or dose of an anti-CD38 antibody or antigen binding fragment thereof described herein such as AB79 together with lenolidomide, dexamethasone, and bortezomib. In other embodiments, the kit may contain multiple doses of an anti-CD38 antibody or antigen binding fragment thereof described herein such as AB79 for subcutaneous administration as well lenolidomide for oral administration, dexamethasone for oral or iv administration, and bortezomib for subcutaneous administration. In one embodiment, the kit may comprise an anti-CD38 antibody or antigen binding fragment thereof formulation described herein prepackaged in a syringe for subcutaneous administration by a health care professional or for home use. In one embodiment, the kit may comprise bortezomib described herein prepackaged in a syringe for subcutaneous administration by a health care professional or for home use.

In certain embodiments, the kit will be for a single administration or dose of an anti-CD38 antibody or antigen binding fragment thereof described herein such as AB79 together with pomolidomide and dexamethasone. In other embodiments, the kit may contain multiple doses of an anti-CD38 antibody or antigen binding fragment thereof described herein such as AB79 for subcutaneous administration as well pomolidomide for oral administration and dexamethasone for oral or iv administration. In one embodiment, the kit may comprise an anti-CD38 antibody or antigen binding fragment thereof formulation described herein prepackaged in a syringe for subcutaneous administration by a health care professional or for home use.

### Articles of Manufacture

In other embodiments, an article of manufacture containing materials useful for the treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in the composition is the antibody. The label on, or associated with, the container indicates that the composition is used for treating the condition of choice. The article of manufacture may further comprise a second container comprising a pharmaceutically-acceptable buffer, such as phosphate-buffered saline, Ringer's solution or dextrose solution. It may further include other materials desirable from a commercial and user standpoint, including other buffers, diluents, filters, needles, syringes, and package inserts with instructions for use.

### EXAMPLES

### Example 1: Summary of Prior Anti-CD38 Antibody Ab79 Clinical Studies

Table 2 provides a summary of clinical studies to date on anti-CD38 antibody AB79.

TABLE 2
AB79 Clinical Studies

| Phase of | Patient | |
|---|---|---|
| Study | Population | Study Title and ID |
| Phase 1 | Healthy | A Phase 1, Randomized, Double-Blind, Placebo- |
| | human | Controlled, Safety, Tolerability and Pharmacokinetic |
| | subjects | Study of Escalating Single Intravenous Infusion (IV) and |
| | | Subcutaneous (SC) Administration of AB79 in Healthy |
| | | Subjects (AB79-101) |
| Phase 1/2a | RRMM | A Phase 1/2a Open-label, Dose-Escalation Study to |
| | | Investigate the Safety and Tolerability, Efficacy, |
| | | Pharmacokinetics, and Immunogenicity of AB79 or |
| | | AB79 + Pomalidomide And Dexamethasone |
| | | Administered Subcutaneously as a Single Agent in |
| | | Patients With Relapsed/Refractory Multiple Myeloma |
| | | (AB79-1501) |
| Phase 1b | NDMM | An Open-Label, Multicenter Phase 1b Study |
| | | Investigating the Safety of TAK-079 in |
| | | Combination With Backbone Regimens for the |
| | | Treatment of Patients With Newly Diagnosed |
| | | Multiple Myeloma and for Whom Stem Cell |
| | | Transplantation Is Not Planned as Initial Therapy |
| | | (AB79-1002) |
| Phase 1b | SLE | A Phase 1b Study to Evaluate the Safety, |
| | | Pharmacokinetics, and Pharmacodynamics of AB79 in |
| | | Combination With Standard Background Therapy in |
| | | Patients With Moderate to Severe Systemic Lupus |
| | | Erythematosus (AB79-2001) |

In the first-in-human (FIH) study, a phase 1, double-blind, placebo-controlled, dose-escalating study (AB79-101) conducted in 74 healthy subjects, AB79 was shown to be safe with no serious adverse events (SAEs) and with expected pharmacodynamic effects. AB79 IV reduced the levels of peripheral blood NK cells >90% from baseline levels in all subjects receiving a single 0.06 mg/kg IV dose, with a Cₘₐₓ of 0.1 μg/mL. AB79 administered SC also reduced the levels of plasmablasts in peripheral blood in a dose-dependent manner. As a potent and convenient second-generation anti-CD38 mAb, AB79 SC warranted therapeutic development for the treatment of multiple myeloma (MM).

The AB79-1501 study is a Phase 1b/2a multicenter, open-label, dose-escalation, single-arm study in patients with relapsed and refractory multiple myeloma (RRMM), who were previously treated with at least a proteasome inhibitor (PI), an immunomodulatory drug (IMiD), an alkylating agent, and a steroid. Patients eligible for study enrollment are refractory or intolerant to at least one PI and at least one IMiD, and have received either ≥3 prior therapies or ≥2 prior therapies, if one of those therapies included a combination of a PI and an IMiD. In the phase 1b dose-escalation part of the study, patients who were previously exposed to an anti-CD38 agent are eligible; however, this criterion is not required. In the phase 2a expansion part of the study, patients were also refractory to at least one anti-CD38 monoclonal therapy at any time during prior treatment. The study was designed to evaluate the safety and tolerability of subcutaneously administered AB79 monotherapy in patients with RRMM, to determine the recommended phase 2 dose (RP2D), and to provide a preliminary assessment of its single-agent activity against RRMM, including in patients who are refractory to daratumumab. Parameters such as safety, tolerability, pharmacokinetics (PK), pharmacodynamics, and disease response were assessed.

Clinical safety data includes that from patients who received a single dose and from patients who received multiple doses in multiple cycles followed by treatment-free periods. Based on the mechanism of action (MOA) of AB79 and the subcutaneous (SC) route of administration, potential adverse events (AEs) included systemic reaction (e.g., cytokine release syndrome (CRS) and hypersensitivity reaction), hematologic effects (e.g., reduced platelet, lymphocyte, neutrophil, and RBC counts), infections (e.g., bacterial and/or viral infections secondary to immune suppression), and injection site reaction (i.e., erythema or tenderness).

### Results

### AB79-1501 Study—AB79 Alone for RRMM

As of the data cutoff, nineteen (19) patients were treated in the dose-escalation portion of the ongoing study in patients with RRMM and have completed at least 1 cycle: 4 patients in the first cohort (45 mg); 3 patients in the second cohort (135 mg); 6 patients in the third cohort (300 mg); and 6 patients in the fourth cohort (600 mg). The most common TEAEs (in ≥10% of patients) regardless of causality in the total population as of this date are fatigue and upper respiratory infection (27% each), insomnia (22%), diarrhea and nausea (17% each), headache and anemia (15% each), neutropenia, abdominal distension, back pain, and hypertension (12% each), cough and pneumonia (10% each). There have been no systemic reactions. Injection site reactions have been rare (<0.25%). The majority of AEs overall (55%) were Grade 1 or 2. In the monotherapy cohorts, no DLTs, have been reported and an MTD has not been identified. The RP2D was determined to be 600 mg with AB79 monotherapy. One drug-related SAE (MedDRA PT: Diverticulitis) was reported in a patient with a past medical history of diverticulitis. Two patients had AEs that led to study discontinuation; both reported as not related to AB79. As of the data cut-off, the preliminary objective response rate (ORR) at the RP2D in anti-CD38 naïve patients who received at least cycle 1 of AB79 was 36% with a clinical benefit rate (defined as minor response or better) of 73% and a disease control rate (defined as stable disease or better) of 91%. Duration of response is not estimable.

### AB79-2001 Study—AB79 Alone for the Treatment of SLE

AB79-2001 is a double-blind, placebo-controlled phase 1b study in patients with moderate to severe SLE. As of the clinical data cutoff, a total of 15 patients had received at least 1 dose of AB79 or placebo. Data is still blinded for this study. No new safety concerns have been identified. No patient had a Grade 3 or higher TEAE or AEs leading to study drug discontinuation regardless if randomized to placebo or AB79.

### Example 2: An Open-Label, Multicenter Phase 1B Study Investigating the Safety of Ab79 in Combination with Backbone Regimens for the Treatment of Patients with Newly Diagnosed Multiple Myeloma (NDMM) and for Whom Stem Cell Transplantation is not Planned as Initial Therapy (AB79-1002)

The primary objective of the study is to determine the recommended phase 2 dose (RP2D) of AB79 when administered to patients with newly diagnosed multiple myeloma (NDMM) in combination with a backbone treatment regimen. The secondary objectives are to determine overall response rate (ORR) and to evaluate safety by assessing incidence of adverse events (AEs).

This is a Phase 1b, open-label, multicenter study to evaluate the safety, efficacy, tolerability, and pharmacokinetics (PK) of AB79 when added to 1 of 2 standard backbone regimens (lenalidomide plus dexamethasone [LenDex] or bortezomib [Velcade] plus lenalidomide and dexamethasone [VRd]) in adult patients with newly diagnosed multiple myeloma (NDMM) for whom stem cell transplantation (SCT) is not planned as initial therapy. The dose and schedule for the backbone regimens (LenDex and VRd) are given in accordance with product labeling or standard medical practice. Treatment cycles are 28 days until disease progression (PD) or unacceptable toxicity occurs. Treatment may be discontinued for other reasons listed below. AB79 is supplied by the sponsor. Bortezomib, dexamethasone, and lenalidomide are standard-of-care agents that are supplied from commercial sources. Approximately 18 adult patients with NDMM for whom SCT is not planned as initial therapy are enrolled in each arm of the study (approximately 36 patients overall).

Patient participation includes a screening phase, a treatment phase, and a follow-up phase. The screening phase is up to approximately 28 days before Cycle 1, Day 1. The treatment phase extends from Cycle 1, Day 1 until patients experience disease progression or unacceptable toxicity or until any other discontinuation criterion is met. The follow-up phase of the study begins once a patient discontinues study treatment and completes the end-of-treatment (EOT) visit; study follow-up continues until the study ends or the patient completes overall survival (OS) follow up.

Once enrolled into the study, patients are assigned to a treatment regimen in a nonrandomized manner. Initially, 6 patients are treated with AB79 in combination with a backbone treatment regimen. Dose-limiting toxicity (DLT) assessment occurs after 6 patients have been given a treatment regimen for 1 cycle. After the first cycle, patients may receive additional cycles of treatment if (1) they have not experienced a DLT, (2) have not shown signs of disease progression, and (3) in the opinion of the investigator, would continue to benefit from additional AB79 added to the backbone regimen. Additional safety reviews are conducted after 6 patients in a given treatment regimen have been treated for 2 and 3 treatment cycles, respectively. When safety data from Cycle 1 is available for all 6 patients in the initial cohort, key safety data is reviewed and evaluated by the sponsor team. Twelve additional patients are then enrolled.

If DLTs are reported in 2 of the 6 patients and it is determined that either a more conservative dose or schedule needs to be evaluated, the sponsor may enroll additional patients to meet study objectives. For example, the sponsor could enroll 6 patients at a more conservative dose or schedule to monitor for safety, and subsequently enroll up to an additional 12 patients to confirm the safety and antimyeloma activity.

Patients are followed for up to 30 days after their last dose of AB79, or until the start of a subsequent alternative anticancer therapy to permit the detection of any delayed treatment related AEs (EOT visit). For patients who discontinue study drug before PD, disease evaluations continue to be performed. After PD is documented, subsequent anticancer treatment and response to treatment is recorded, and survival status is obtained. If the patient dies, the date and cause of death is collected and documented. Follow-up continues until the study ends.

The analyses for the clinical study report are conducted after all patients enrolled in the study have had the opportunity to complete 2 years of therapy. The study is designed to last 36 months (including enrollment, treatment and follow-up periods).

### Study Design

300 mg of AB79 is administered subcutaneously once weekly for 8 weeks (8 doses), once every 2 weeks for 16 weeks (8 doses), and once every 4 weeks thereafter until PD (in combination with backbone therapy). Backbone therapies (LenDex or VRd) are dosed in accordance with product labeling/local institutional practices. VRd is given with bortezomib weekly×3 weeks and LenDex in a standard 28 day cycle. The treatment schedules are shown in Table 3. Patients are evaluated from first dose of AB79 until 30 days after PD or until protocol-defined treatment discontinuation criteria are met.

TABLE 3
Treatment Schedules

| | | | | | | | | | | | | EOT | |
|---|---|---|---|---|---|---|---|---|---|---|---|---|---|
| Treatment Period | | | | | | | | | | | | Up to | PFS |
| Cycle | | | | | | | | | | | | 30 | Every |
| Cycles | | | | Cycles | | | | Cycles 7 and | | | | Days | 4-12 |
| 1 and 2 | | | | 3 to 6 | | | | Beyond | | | | After | Weeks |
| Days | | | | | | | | | | | | Last | Until |
| 1 | 8 | 15 | 22 | 1 | 8 | 15 | 22 | 1 | 8 | 15 | 22 | Dose | PD |

| | Window | | |
|---|---|---|---|
| Study | | +1 | ±1 |
| Procedure | ±2 Days | Week′ | Week |

Study Dosing with AB79 Only

| | | | | | | | | | |
|---|---|---|---|---|---|---|---|---|---|
| Preinjection | X | X | X | X | X | | X | | X |
| medication^{a} | | | | | | | | | |
| AB79, SC^{b} | X | X | X | X | X | | X | | X |
| Dexamethasone, | X | X | X | X | X | X | X | X | X |
| PO^{c} | | | | | | | | | |

Study Dosing with AB79 and LenDex Regimen

| Preinjection | X | X | X | X | X | | X | | X | | | |
|---|---|---|---|---|---|---|---|---|---|---|---|---|
| medication^{a} | | | | | | | | | | | | |
| AB79, SC^{b} | X | X | X | X | X | | X | | X | | | |
| Lenalidomide, | Days 1 to 21 in each cycle | | | | | | | | | | | |
| PO | | | | | | | | | | | | |
| Dexamethasone, | X | X | X | X | X | X | X | X | X | X | X | X |
| PO^{c} | | | | | | | | | | | | |

Study Dosing with AB79 and VRd Regimen

| Preinjection | X | X | X | X | X | | X | | X | | | |
|---|---|---|---|---|---|---|---|---|---|---|---|---|
| medication^{a} | | | | | | | | | | | | |
| AB79, SC^{b} | X | X | X | X | X | | X | | X | | | |
| Lenalidomide, | Days 1 to 21 in each cycle | | | | | | | | | | | |
| PO | | | | | | | | | | | | |
| Bortezomib, | X | X | X | | X | X | X | | X | X | X | |
| SC^{d} | | | | | | | | | | | | |
| Dexamethasone, | X | X | X | X | X | X | X | X | X | X | X | X |
| PO^{c} | | | | | | | | | | | | |

Study Dosing with AB79 and PomDex Regimen

| Preinjection | X | X | X | X | X | | X | | X | | | |
|---|---|---|---|---|---|---|---|---|---|---|---|---|
| medication^{a} | | | | | | | | | | | | |
| AB79, SC^{b} | X | X | X | X | X | | X | | X | | | |
| Pomalidomide, | Days 1 to 21 in each cycle | | | | | | | | | | | |
| PO | | | | | | | | | | | | |
| Dexamethasone, | X | X | X | X | X | X | X | X | X | X | X | X |
| PO^{c} | | | | | | | | | | | | |

The strength of the AB79 drug product for SC is 100 mg AB79 in 1 mL (100 mg/mL). After patients have received premedication treatment, AB79 doses are administered with syringes as SC injections up to a maximum volume of approximately 2 mL per injection (i.e., 200 mg/2 mL). The injection sites are rotated, using the abdomen, thighs, arms, and upper buttock area.

### Lenalidomide-Dexamethasone Regimen (LenDex)

Lenalidomide is administered orally at 25 mg daily for 21 days in accordance with product labeling. Dexamethasone is administered intravenously (IV) or orally at 40 mg weekly, or 20 mg weekly if the patient is >75 years old, in accordance with product labeling. Dexamethasone is taken before AB79 dosing as a premedication for at least Cycle 1; if there are no systemic IRRs, then the timing of dexamethasone dosing may be adjusted per standard medical judgment. Dexamethasone after Cycle 8 is dosed per dexamethasone associated tolerability and physician medical judgement. The treatment cycle is 28 days as per product labeling until disease progression or unacceptable toxicity. Lenalidomide and dexamethasone are obtained from commercial sources.

### Bortezomib-Lenalidomide-Dexamethasone Regimen (VRd)

Bortezomib is administered SC at 1.3 mg/m² weekly (Days 1, 8, and 15) for a maximum of 8 cycles in accordance with product labeling. Lenalidomide is administered orally at 25 mg daily for 21 days in accordance with product labeling. Dexamethasone is administered intravenously or orally at 40 mg weekly, or 20 mg weekly if the patient is >75 years old, in accordance with product labeling. Dexamethasone is taken before AB79 dosing as a premedication for at least Cycle 1; if there are no systemic IRRs, then the timing of dexamethasone dosing may be adjusted per standard medical judgment. Dexamethasone after Cycle 8 is dosed per dexamethasone associated tolerability and physician medical judgement. The treatment cycle is 28 days as per product labeling until disease progression or unacceptable toxicity (note 8 cycle maximum for bortezomib). Bortezomib, lenalidomide, and dexamethasone are obtained from commercial sources.

### Predose Medication

Before each injection, patients receive the following premedication approximately 1 to 3 hours before the AB79 injection on each dosing day: oral acetaminophen (650 to 1000 mg) and oral or IV diphenhydramine (25 to 50 mg, or equivalent). Any patient with a history of COPD may receive premedication with montelukast 10 mg (or an equivalent leukotriene inhibitor). Postinfusion medications, such as short- and long-acting bronchodilators and inhaled corticosteroids, may be administered. After the first 4 injections, if the patient experiences no major infusion reaction, these additional inhaled postinfusion medications may be discontinued. The investigator may reduce predose and postdose medications if, after the first 4 injections, the patient experiences no major infusion reaction.

### Postdose Medications

Corticosteroid cream is applied topically to injection site(s) and ice is applied locally for approximately 10 to 15 minutes. Patients may receive low-dose methylprednisolone (<20 mg) for the prevention of delayed injection-related reactions as clinically indicated after an injection.

### Main Criteria for Inclusion

Each patient must meet all the following inclusion criteria to be enrolled in the study: (1) Previously untreated MM as defined by the International Myeloma Working Group (IMWG) criteria requiring treatment according to the investigator; (2) Patients are appropriate candidates for either the VRd or Len Dex backbone antimyeloma therapy according to the investigator; (3) Patients have measurable disease defined by at least 1 of the following: (a) Serum M-protein ≥1 g/dL (≥10 g/L); (b) Urine M-protein ≥200 mg/24 hr; and (c) Serum free light chain (FLC) assay: involved FLC level ≥10 mg/dL (≥100 mg/L) provided the serum FLC ratio is abnormal; (4) Adult male or female patients 18 years of older not expected to undergo SCT as initial therapy. Stem cell harvest and mobilization regimen is acceptable if clinically indicated but must first be confirmed by the clinician/designee. Stem cell mobilization and harvest may occur at any time after the fourth treatment cycle according to institutional clinical practice; (5) Patients meet the following laboratory criteria: (a) Hemoglobin >7.5 g/dL; (b) Absolute neutrophil count (ANC) ≥1000/mm³ (Granulocyte-colony stimulating factor (G-CSF) or other growth factors to help patients meet eligibility criteria are not allowed); (c) Platelet count ≥75,000/mm³ (Platelet transfusions to help patients meet eligibility criteria are not allowed); (d) Total bilirubin ≤1.5 times the upper limit of the normal (ULN) (except for Gilbert syndrome: direct bilirubin ≤2 times the ULN); (e) Alanine aminotransferase (ALT) and aspartate aminotransferase (AST)≤3 times the ULN; (f) Creatinine clearance, by calculated creatinine clearance, ≥50 mL/minute; (6) Patients practice contraception or abstinence; (7) For patients receiving lenalidomide: must be able to take concurrent prophylactic anticoagulation per standard clinical practice as directed by the investigator; (8) Life expectancy >3 months; and (9) Eastern Cooperative Oncology Group (ECOG) performance status score ≤2; and (10) Voluntary written informed written consent must be given before performance of any study related procedure not part of standard medical care with the understanding that consent may be withdrawn at any time without prejudice to future medical care; and (11) Patient willing and able to adhere to the standard medical procedures for multiple myeloma, the study visit schedule and other protocol requirements.

### Main Criteria for Evaluation and Analyses:

The primary endpoint is the recommended dose of the combination of AB79 and the backbone regimen, based on the number of patients with DLTs by Medical Dictionary for Regulatory Activities (MedDRA) in Cycle 1. The secondary endpoints are (a) ORR for each regimen based on investigator's assessment (response of partial response (PR) or better) based on IMWG criteria; (b) Incidence of AEs by MedDRA System Organ Class and Preferred Term, to include Grade 3 or higher events, serious adverse events (SAEs), AEs, leading to AB79 discontinuation, and AEs resulting in on-study deaths.

### Statistical Considerations:

Adverse events are summarized by treatment group and overall. Categorical variables such as ORR are tabulated by treatment group and overall. Time to event variables such as DOR, PFS and OS are analyzed using Kaplan-Meier survival curves, and Kaplan-Meier medians (if estimable) are provided. PK parameters are summarized as appropriate.

### Sample Size Justification:

The sample size is not determined based on formal hypothesis testing but instead is based on outcomes of DLT and safety evaluations. As such, each regimen of AB79 and protocol-defined backbone therapy is evaluated individually for DLT determination and safety. Initially, 6 DLT evaluable patients is evaluated for safety and DLTs before additional patients are enrolled. A cohort may be expanded by enrolling additional patients to obtain more comprehensive assessment of safety, PK, pharmacodynamics, or disease response, and to further inform the selection of the RP2D. Once the RP2D has been determined, up to an additional 12 patients (total of approximately 18 patients for each regimen of AB79 and backbone therapy) is enrolled. No prospective calculations of statistical power have been made; however, Table 4 shows the width of the 80% confidence interval, based on the observed ORR in a cohort size of 18 patients, for a range of observed response rates.

TABLE 4
A Summary of 80% CI Based on the Observed ORR

| Observed Rate | | | | | |
|---|---|---|---|---|---|
| of Response | 11% (2/18) | 22% (4/18) | 33% (6/18) | 44% (8/18) | 56% (10/18) |
| 80% CI (n = 18) | (3.0-26.9) | (10.1-39.6) | (18.6-51.2) | (27.9-62.0) | (38.0-72.1) |

ORR: observed response rate. The observed rate of response is given as a percentage (n with response/N).

### Definitions of DLT

Toxicity is evaluated according to the NCI CTCAE (version 4.03, effective 14 Jun. 2010; US Department of Health and Human Services, 2010). DLTs are evaluated at the end of Cycle 1. Only toxicities that occur during the DLT evaluation period are used for the purposes of defining DLT and for subsequent cohort expansion or dose-modification decisions. DLTs are based on AB79-related toxicities. If noncompliance with protocol-defined requirements (e.g., antiviral prophylaxis) results in toxicities of ≥Grade 3, these toxicities do not qualify as DLTs. TEAEs that are clearly due to extraneous causes will not be defined as DLTs. A DLT is defined as any of the following events that are considered by the investigator to be at least possibly related to AB79: (1) Hematologic toxicity, clearly unrelated to the underlying disease, are defined as follows: (a) Grade 4 thrombocytopenia (platelet count <25,000/mm³) lasting more than 7 consecutive days, or ≥Grade 3 low platelet count with significant bleeding where clinically significant bleeding is defined as a blood loss of 100 mL or the requirement for a red blood cell transfusion; (b) A platelet count <10,000/mm3; (c) Grade 4 neutropenia (ANC<500 cells/mm³) lasting more than 7 consecutive days; (d) Grade 3 neutropenia (ANC<1000 cells/mm³) with infection and/or fever where fever is defined as a single temperature >38.5° C. or a sustained temperature >38° C. for >1 hour); and (e) Grade ≥3 hemolysis, except those events that are clearly due to extraneous causes (e.g., negative direct Coombs test), are included in the DLT definition; (2) Nonhematologic toxicity of Grade 3 or higher clearly unrelated to the underlying disease, with the exception of: (a) Grade 3 injection-associated (systemic) reaction (IAR) that responds to symptomatic treatment (e.g., antihistamines, nonsteroidal anti-inflammatory drugs, narcotics, IV fluids), without recurrence of Grade 3 symptoms; (b) Grade 3 fatigue or asthenia lasting for <7 days after the last administration of AB79; (c) Grade 3 nausea or Grade 3 vomiting that responds to antiemetic treatment. Optimal antiemetic prophylaxis is defined as an antiemetic regimen that employs a 5-hydroxytryptamine type 3 antagonist (5-HT3) given in standard doses and according to standard schedules; (d) Grade 3 diarrhea that responds to antidiarrheal treatment; and (e) Isolated Grade ≥3 elevation of ALT or AST that resolves to Grade ≤1 or baseline, within 7 days.

An incomplete recovery from treatment-related toxicity causing a >2-week delay in the next scheduled AB79 injection before the initiation of Cycle 2 is considered a DLT. Inability to give at least 80% of the planned doses of the individual agents in the backbone regimen due to drug-related AE are evaluated with all available safety data as a possible DLT before cohort expansion decisions.

### Dose Escalation Rules

Initially, 6 patients are treated at the initial dose of AB79 in combination with a backbone treatment regimen: LenDex or VRd. DLT determination and safety assessment will occur after 6 DLT evaluable patients receiving a given AB79 regimen with the backbone treatment have completed 1 full cycle. When safety data are available for all 6 patients in the cohort, key safety data are reviewed and evaluated before enrolling additional patients. If DLT is observed in 1 or less patient in a treatment regimen, at least 12 additional patients are enrolled in that treatment regimen to validate the safety of the AB79 dose. If DLT is observed in 2 or more of 6 patients, the dose of AB79 is de-escalated at a dose and/or schedule determined by the sponsor, and 6 additional patients are treated before an expansion to 12 additional patients; a more conservative dose schedule may also be implemented as a means to provide an overall lower dose. Each regimen of AB79 plus backbone regimen is evaluated individually for DLT determination and safety.

Patients not receiving all doses of AB79 in Cycle 1 for reasons other than DLTs are replaced within the cohort. Patients receiving all doses of AB79, yet for unforeseen circumstances recovery of toxicity is not available and safety in Cycle 1 cannot be fully evaluated should be replaced within the cohort. Patients experiencing a DLT should not be replaced.

For all patients, additional ongoing safety reviews are conducted after 6 patients in a given backbone regimen have been treated for 2 and 3 treatment cycles, respectively. Evaluation of intermediate doses or doses up to that evaluated and found safe in the RRMM study, alternative dosing schedules (dosing interval), and expansion of an existing dose level are all permissible following discussions between the sponsor and the investigators, if such measures are needed for patient safety or for a better understanding of the dose-related toxicity, exposure, or pharmacodynamics of AB79.

### Safety and Disease Assessments

Safety evaluations include monitoring for TEAEs in accordance to the National Cancer Institute Common Terminology Criteria for Adverse Events (NCI CTCAE), version 4.03. Changes in clinical laboratory parameters (standard hematology and chemistry), vital sign, electrocardiogram (ECG) monitoring, and Eastern Cooperative Oncology Group (ECOG) performance status that are judged by the investigator as clinically significant are recorded both on the source documentation and in the electronic case report (eCRF) as an AE. Toxicities that occur during the DLT evaluation period are used for the purposes of defining DLT and for subsequent cohort expansion or dose modification decisions. DLTs are based on AB79-related toxicities.

Efficacy assessment of tumor response and disease progression are conducted in accordance with IMWG criteria. Efficacy evaluations include measurements of myeloma proteins in serum and urine. Bone marrow examinations, skeletal surveys, computed tomography (CT) or magnetic resonance imaging (MM) to evaluate lytic and/or extramedullary plasmacytomas, and serum calcium corrected for albumin are evaluated as necessary based on patient's baseline disease status.

### PK, Pharmacodynamic, and Immunogenicity Assessments

Blood samples for PK, pharmacodynamic, and immunogenicity (including antidrug antibody (ADA)) testing are collected at certain time points.

### Primary Endpoints

The primary endpoint is a recommended dose of the combination of AB79 and the backbone regimen based on the number of patients with dose-limiting toxicity (DLT) by Medical Dictionary for Regulatory Activities (MedDRA) in Cycle 1.

### Secondary Endpoints

The secondary endpoints are: (a) ORR for each regimen based on investigator's assessment (response of at least partial response [PR]) based on IMWG criteria; and (b) Incidence of AEs by MedDRA System Organ Class and Preferred Term, including Grade 3 or higher events, serious adverse events (SAEs), AEs leading to AB79 discontinuation, and AEs resulting in on-study deaths.

### Exploratory Endpoints

The exploratory endpoints are: (1) One-year estimate of PFS, defined as Kaplan-Meier estimate of patients free of progression or death at 1 year from date of first dose; (2) Duration of response, defined as the time from the date of the first documentation of response to the date of the first documented PD; (3) Time to response, defined as the time from the date of the first dose to the date of the first documentation of response (PR or better); (4) One-year estimate of survival, defined as the patient survival probability at 1 year after the date of the first dose of treatment; (5) OS, defined as the time from the date of first dose of treatment to date of death; (6) Determination of MRD within bone marrow aspirate (BMA) samples obtained at assessment of suspected VGPR or better (using next-generation flow cytometry); (7) PK of AB79 in combination with the backbone treatment regimen. PK parameters including but not limited to Cₘₐₓ, time after administration at which maximum plasma concentration is reached (Tₘₐₓ), and area under the curve (AUC); (8) CD38 expression changes on MM cells and other immune cells in BMA and peripheral blood before, during, and at the end of therapy; (9) Pharmacodynamic analysis of the presence and changes of immune cells in BMA and peripheral blood before, during, and at the end of therapy; (10) Exploratory evaluation of potential biomarkers predictive of response and/or resistance including but not limited to changes in cytokines/chemokines; (11) Comparison of change in global health status between baseline and each postbaseline assessment, as measured by the global health scale, functioning, and symptoms of EORTC QLQ-C30 and EORTC QLQ-MY20; and (12) Anti-AB79 antibody incidence and characteristics.

### Criteria for Dose Modification of Standard Backbone Agents

Patients receiving bortezomib and lenalidomide may have the respective drug modified according to the prescribing information. Table 5 suggests dose reduction steps that align with the VRd and LenDex backbone regimens. Patients experiencing AEs attributed to one of these agents should be reduced by 1 dose level as noted in the prescribing information. When a dose reduction of one of these agents is required because of toxicity, no dose re-escalation is permitted.

TABLE 5
Suggested Dose Modifications for VRd and
Len Dex Backbone Therapy Regimens

| Dose | Bortezomib | Lenalidomide |
|---|---|---|
| Starting dose | 1.3 mg/m² | 25 mg |
| Dose level-1 | 1.0 mg/m² | 15 mg |
| Dose level-2 | 0.7 mg/m² | 10 mg |
| Dose level-3 | Discontinue | 5 mg |
| Dose level-4 | | 2.5 mg; discontinue if 2.5 mg |
| | | dose is not tolerated |

Dose modification of these agents as aligned with prescribing information medical judgment.

### Dexamethasone Treatment Modifications (Both Arms)

Patients experiencing AEs attributed to dexamethasone may have doses of dexamethasone reduced according to standard medical judgment. When a dose reduction is required because of toxicity, no dose re-escalation is permitted.

Tables 6-9 provide a listing of the standard of care laboratory tests and research tests.

TABLE 6
Clinical Hematology and Chemistry: Standard of Care Laboratory Tests

| Hematology | Chemistry | |
|---|---|---|
| Leukocytes with complete | Albumin | CO₂ (bicarbonate) |
| differential (total neutrophils | | |
| [ANC], lymphocytes, monocytes, | | |
| eosinophils, and monocytes) | | |
| Platelet count | Alkaline | Creatinine |
| | phosphatase | |
| Hemoglobin | ALT | Calculated |
| | | Creatinine Clearance |
| Serum pregnancy test | AST | Glucose |
| | β2- | Lactate |
| | microglobulin | dehydrogenase |
| | Bilirubin (direct | Potassium |
| | and indirect) | |
| | Calcium | Sodium |
| | Chloride | Urate |

ALT: alanine aminotransferase; ANC: absolute neutrophil count; AST: aspartate aminotransferase.

TABLE 7
Clinical Hematology and Chemistry: Tests for Research Purposes

| Clinical Hematology or Chemistry | Serology Antibody Titers |
|---|---|
| Coagulation Panel (PT, PTT, INR) | HBV |
| Indirect and direct Coombs | HCV |
| C-reactive protein | HIV |

HBV: hepatitis B virus; HCV: hepatitis C virus; INR: international normalized ratio; PT: prothrombin time; PTT: partial thromboplastin time.

TABLE 8
Clinical Urinalysis: Tests for Research Purposes
Urinalysis

| | |
|---|---|
| Bilirubin | pH |
| Glucose | Protein |
| Ketones | Specific gravity |
| Leukocytes | Turbidity, appearance, and color |
| Nitrite | Urobilinogen |
| Occult blood | Microscopic assessment ^{a} |

RBC: red blood cell; WBC: white blood cell.
^{a} Microscopic analyses are performed only as clinically indicated: bacteria, RBCs, WBCs, casts, and crystals.

For estimation of creatinine clearance, the Cockcroft-Gault formula is employed as follows: Estimated creatinine clearance=[(140−Age)×Mass (kg)]/72×serum creatinine (mg/dL)]. For female patients, the result of the formula above is multiplied by 0.85.

### Disease Assessment

Patients are assessed for disease response according to the IMWG criteria.

TABLE 9
Myeloma Disease Assessments: Standard of Care Tests

| Serum/Urine | Bone Marrow/Imaging |
|---|---|
| SPEP | Bone marrow biopsy and/or aspirate ^{a} |
| UPEP | Cytogenetics [presence of del(17), t(4:14), |
| | and t(14:16) at a minimum] |
| Immunofixation (serum | Imaging (skeletal survey, CT, |
| and urine) | PET/CT, MRI) |
| Quantification | |
| immunoglobulin levels | |
| Serum FLC | |

^{a} A clinically indicated BMA drawn prior to consent is acceptable for the baseline assessment provided that it is collected within 8 weeks of study entry if acceptable results are available for morphology, clinical staging, and cytogenetics. BMA samples obtained during Cycle 2 Day 1, Cycle 4 Day 1, Cycle 7 Day 1, and Cycle 13 Day 1 are for research purposes, unless the timing of the sample aligns with a suspected CR. In such instances, the sampling procedure and analysis would be standard of care. BMA: bone marrow aspirate; CR: complete remission; CT: computed tomography; FLC: free light chains; MRI: magnetic resonance imaging; PET: positron emission tomography; SPEP: serum protein electrophoresis; UPEP: urine protein electrophoresis.

### Clinical Laboratory Evaluations for Disease Assessments

A blood sample is collected during screening for measurement of serum β2-microglobulin and albumin for determination of disease stage according to the International Staging System. Clinical laboratory evaluation for disease assessments, serum protein electrophoresis (SPEP), 24-hour urine collection for urine protein electrophoresis (UPEP), serum FLC, serum and urine immunofixation testing, and total immunoglobulin levels are obtained. If the patient has measurable M protein restricted to the urine, M-protein component quantification can be determined by UPEP only. Patients measurable by SPEP only have 24-hour urine collected at screening and EOT and to document PR, VGPR, CR, or PD. Immunofixation is done to confirm CR.

### Interference Testing

As AB79, similar to daratumumab, is a monoclonal IgG kappa antibody, the SPEP and serum immunofixation can be positive due to anti-CD38 monoclonal antibody. Therefore, whenever the SPEP values reach ≤0.2 g/dL for 2 consecutive disease evaluations, a CR should be suspected triggering the need for interference testing. Currently, if the interference test results are positive, then the assay is considered positive for endogenous protein, and thus there is still disease present. If the interference test results are negative, then the assay is considered negative for endogenous protein, and thus the remaining protein is likely CD38 monoclonal antibody. A confirmatory bone marrow aspirate (BMA) evaluation for possible CR may be performed is not done already.

Blood samples for IgM, IgG, and IgA are obtained at screening and throughout the study at certain time points. Quantitative IgD and IgE are done at screening only. For the rare patient with documented IgD or IgE MM, the quantitative test for that antibody is followed at the same time points as IgG and IgA.

Bone Marrow Biopsy and/or Aspirate

BMA and/or biopsy results (from bone marrow done within 8 weeks of study entry) for evaluation of morphology, clinical staging, and cytogenetics must be available at screening, if not, a BMA and/or biopsy is obtained at screening. If not previously assessed, a BMA is performed for at least the following cytogenetic abnormalities: deletion of chromosome 17 [del(17)], translocation of chromosome 4:14 [t(4:14)], and translocation of chromosome 14:16 [t(14:16)]. If a BMA is done during screening, samples are tested for baseline CD38 expression, baseline for receptor occupancy, pharmacodynamic measurements, and immunoprofiling.

Patients suspected of achieving a CR have a BMA collected at any time to document CR as per IMWG criteria. Suspected CR (sCR) is defined independently of the immunofixation result; BMA is performed when the M-protein measurement in SPEP (for heavy-chain patients) or UPEP (for light-chain patients) becomes below detection limits or nonquantifiable. The BMA sample is evaluated for CR and MRD analyses. Determination of the kappa/lambda ratio by immunohistochemistry or immunofluorescence must be performed to assess for sCR.

### Cytogenetics/Fluorescence In Situ Hybridization

Patients who do not have historically documented cytogenetic results for the high-risk abnormalities of del(17), t(4:14) and t(14:16) have cytogenetic evaluation performed on the BMA sample at screening. If historically documented cytogenetics are available, a BMA sample at screening is not required as long as results on the minimal cytogenetic markers noted here are available. Cytogenetic evaluation using fluorescence in situ hybridization or conventional cytogenetics (karyotype) is acceptable. However, at a minimum, cytogenetic markers must include the 3 high-risk abnormalities of del(17), t(4:14) and t(14:16). Additional abnormalities [ampl 1q, del (13), or del (1p)] may also be tested.

### Radiographic Assessment of Disease

Imaging to evaluate lytic and extramedullary disease is performed at a minimum at screening and at the EOT visit. The choice of imaging modality (e.g., skeletal survey, CT, MM, positron emission tomography-computed tomography [PET-CT]), is at the discretion of the investigator; however, all treatment phase and follow-up scans should use the same imaging modality used at screening to facilitate consistent disease assessment. Imaging tests are done at screening (within 8 weeks of the first dose of study drug). If soft tissue extramedullary disease is documented, repeat imaging should be performed as required to document response or progression as per IMWG criteria.

### Biomarker, Pharmacodynamic, and PK Samples

Table 10 provides a list of patient samples that are collected for study.

TABLE 10
Primary Specimen Collection

| Specimen Name in Schedule | Primary | Description of |
|---|---|---|
| of Procedures | Specimen | Intended Use |
| BMA sample for cytogenetics ^{a} | BMA | Cytogenetics |
| BMA Sample for Minimal | BMA | Minimal residual |
| Residual Disease and | | disease, |
| Immunoprofiling | | pharmacodynamics and |
| | | immunoprofiling |
| Blood Sample for | Blood | (Pharmacodynamic and |
| Pharmacodynamic and | | immune cell changes) |
| Immunoprofiling | | |
| Measurements | | |
| Serum Sample for Circulating | Serum | Biomarker |
| Biomarkers | | Measurements |
| Serum Sample for | Serum | Immunogenicity |
| Immunogenicity | | assessments |
| Serum Sample for AB79 PK | Serum | PK Measurements |

Serum samples for the measurement of concentrations of AB79 are collected at multiple time points. The timing, but not the total number, of samples may be modified during the study on the basis of emerging PK data if a change in the sampling scheme is considered necessary to better characterize the PK profile of AB79.

Several biomarkers are assessed to test for correlation with safety, PK, and, if possible, with efficacy. These biomarkers are used to identify patients who have a higher probability of response or adverse reactions to AB79. The biomarker sample analysis is performed if or when required. Because new techniques continue to be developed, the methods recommended for the biomarker analysis cannot be anticipated.

BMA samples are collected for assessing MRD and to profile tumor and immune cells present in the bone marrow. BMA samples are also collected to analyze CD38 expression and monitor changes in immune cells by flow cytometry during and at the end of treatment.

Serum samples for cytokine/chemokine levels, for example, are collected before, during, and at the end of treatment to help identify patients who have a higher probability of response or of experiencing adverse reactions to AB79.

Blood samples are collected to analyze CD38 expression and monitor changes in immune cells by flow cytometry before, during and at the end of treatment. Blood samples are also collected for profiling of immune cells before, during, and at the end of treatment and analyzed for the presence and changes of immune cells by flow or mass cytometry. Blood samples for the assessment of ADA are collected at various time points. Samples must be collected before study drug is administered on a dosing day, and optionally at unscheduled visits for a subject who experiences an AE considered by the investigator to be consistent with hypersensitivity or other IRR. A sample may be further characterized if a positive ADA is detected.

Discontinuation of Treatment with Study Drug and Patient Replacement

Study therapy is permanently discontinued for patients meeting any of the following criteria: withdrawal by subject; pregnancy; AE/SAE; PD; unsatisfactory therapeutic response; initiation of hematopoietic SCT; protocol deviation; study terminated by sponsor; lost to follow-up; and physician decision. Once study therapy has been discontinued, all study procedures outlined for the EOT visit are completed.

Note that some patients may discontinue study therapy for reasons other than PD before completing the full treatment course; these patients will remain in the study for PFS follow-up assessments until PD occurs. Unless the patient withdraws consent to follow-up, PFS and/or OS follow-up assessments will continue to be conducted.

Patients remaining on study therapy at the time of study closure (whether completion of the study or any other reason) are provided continued access to AB79, either through commercial drug supply (where available and reimbursable) or through continued treatment in another extension or rollover study.

Withdrawal of Patients from Study

A patient is withdrawn from the study for any of the following reasons: death; study, terminated by sponsor; withdrawal by subject; and lost to follow-up.

### Adverse Events

### Pretreatment Event Definition

A pretreatment event is any untoward medical occurrence in a patient or subject who has signed informed consent to participate in a study but before administration of any study medication; it does not necessarily have to have a causal relationship with study participation.

### Adverse Event (AE) Definition

AE means any untoward medical occurrence in a patient or subject administered a pharmaceutical product; the untoward medical occurrence does not necessarily have a causal relationship with this treatment. An AE can therefore be any unfavorable and unintended sign (including an abnormal laboratory finding), symptom, or disease temporally associated with the use of a medicinal (investigational) product whether or not it is related to the medicinal product. This includes any newly occurring event or a previous condition that has increased in severity or frequency since the administration of study drug. An abnormal laboratory value will not be assessed as an AE unless that value leads to discontinuation or delay in treatment, dose modification, therapeutic intervention, or is considered by the investigator to be a clinically significant change from baseline.

### Serious Adverse Event (SAE) Definition

SAE means any untoward medical occurrence that at any dose: (1) Results in death; (2) Is life-threatening (refers to an AE in which the patient was at risk of death at the time of the event. It does not refer to an event which hypothetically might have caused death if it were more severe); (3) Requires inpatient hospitalization or prolongation of an existing hospitalization; (4) Results in persistent or significant disability (defined as a substantial disruption of a person's ability to conduct normal life functions) or incapacity; (5) Is a congenital anomaly/birth defect; (6) Is a medically important event. An AE that not result in death, be immediately life-threatening, or require hospitalization, but may be considered serious when it may jeopardize the patient, require medical or surgical intervention to prevent one of the outcomes listed above, or involves suspected transmission via a medicinal product of an infectious agent. Examples of such medical events include allergic bronchospasm requiring intensive treatment in an emergency room or at home, blood dyscrasias or convulsions that do not result in inpatient hospitalization, or the development of drug dependency or drug abuse; any organism, virus, or infectious particle (e.g., prion protein transmitting transmissible spongiform encephalopathy), pathogenic or nonpathogenic, is considered an infectious agent.

Intensity for each AE, including any lab abnormality, is determined using the NCI CTCAE, version 4.03, effective 14 Jun. 2010. Clarification should be made between an SAE and an AE that is considered severe in intensity (Grade 3 or 4) because the terms serious and severe are not synonymous. The general term severe is often used to describe the intensity (severity) of a specific event; the event itself, however, may be of relatively minor medical significance (such as a Grade 3 headache). This is not the same as serious, which is based on patient/event outcome or action criteria described above and is usually associated with events that pose a threat to a patient's life or ability to function. A severe AE (Grade 3 or 4) does not necessarily need to be considered serious. For example, a white blood cell count of 1000/mm³ to less than 2000/mm³ is considered Grade 3 (severe) but may not be considered serious. Seriousness (not intensity) serves as a guide for defining regulatory reporting obligations.

### Monitoring of AEs and Period of Observation

AEs, both nonserious and serious, are monitored throughout the study as follows: (1) AEs are reported from the signing of informed consent through 30 days after administration of the last dose of study drug. AEs ongoing at EOT are monitored until they are resolved, return to baseline, are clearly determined to be due to a patient's stable or chronic condition or intercurrent illness(es), the start of second-line alternative therapy, or 6 months after PD has occurred whichever comes first; (2) SAEs are reported from the signing of informed consent through 30 days after administration of the last dose of study drug. After this period, only SAEs related to Takeda agents (AB79 and Velcade) must be reported to the Takeda Global Pharmacovigilance department or designee. SAEs are monitored until they are resolved or are clearly determined to be due to a patient's stable or chronic condition or intercurrent illness(es). In addition, because of the increased risk of new primary malignancies with lenalidomide, all cases of new primary malignancy are reported irrespective of causality to the study treatment regimen, from the time of first dose of the study treatment regimen through death (including the follow-up periods), until termination of the study by the sponsor, or for a minimum of 3 years after the last dose of any of the drugs within the study treatment regimen, whichever comes first.

### PK Analysis

PK parameters are estimated using noncompartmental analysis methods. Parameters are calculated for individual patients included in the PK analysis set using the AB79 concentration-time data. The calculated PK parameters will include, but not be limited to, Cₘₐₓ, tₘₐₓ, and AUCₗₐₛₜ.

PK parameters are summarized using descriptive statistics. Individual AB79 concentration-time data and individual PK parameters are presented in listings and tabulated using summary statistics by dose cohort. Individual and mean concentration-time profiles are plotted by dose cohort. The PK data collected in this study may also contribute to future population PK analyses of AB79. These population PK analyses may include data collected in other AB79 clinical studies. The analysis plan for the population PK analysis are separately defined, and the results of these analyses are reported separately. Similarly, the time-matched PK and triplicate ECG data collected in this study may contribute to future concentration-QT interval corrected for heart rate (QTc) analyses. These analyses may include data collected in other AB79 clinical studies. The analysis plan for the concentration-QTc analysis is separately defined, and the results are reported separately.

### Pharmacodynamic Analysis

During the clinical development of AB79, several biomarkers are assessed to test for their correlation with safety and, if possible, with efficacy. Markers that are studied are markers linked either to the drug itself or to the treated disease. Markers indicating changes in tumor burden (i.e., changes in immune cells, or changes in soluble biomarkers) are summarized using descriptive statistics. Individual data are listed. Summaries are provided separately for each study phase and by dose, as applicable.

### PK/Pharmacodynamic Analysis

Attempts are made to evaluate the potential relationships between AB79 dose and AB79 serum exposure versus several biomarkers, such as CD38 expression level, and changes in immune cells. These analyses are exploratory in nature, and all results are descriptive in nature.

### Immunogenicity Analyses

AB79 immunogenicity status (ADA negative, transient, and persistent positive and ADA titer) are analyzed and summarized using descriptive statistics as applicable (based on the SAP). The effect of immunogenicity on PK, safety, and efficacy are explored. Immunogenicity analyses are based on available data from patients with a baseline assessment and at least 1 postbaseline immunogenicity assessment.

### QOL Analysis

QOL is assessed by the EORTC 30-item QLQ-C30 test and by EORTC QLQ-MY20 (20-item assessment) specifically designed to address the QOL for those with MM (http://groups.eortc.be/qol/questionnaires Accessed 19 Mar. 2019) The primary constructed scales from the EORTC QLC-C30 are: Global Health Status/QOL, Physical Functioning, Role Functioning, Emotional Function, Cognitive Functioning, and Social Functioning. Nine additional scales can be derived: Fatigue Nausea and Vomiting, Pain, Dyspnea, Insomnia, Appetite Loss, Constipation, Diarrhea, and Financial Difficulties. Both surveys are administered before at Cycle 1 Day 1, then every 3 months in Year 1, and every 6 months thereafter until PD.

Cycle 1, Day 1 QOL measures are used as baseline. The analysis is performed on summary scores as well as on subscales and individual symptoms. The change between baseline and each postbaseline assessment is described overall. The QOL endpoints are the global health status and the remaining EORTC QLQ-C30 and EORTC QLQ-MY20 subscale and individual item scores. The changes in scores are presented using cumulative frequency distribution figures.

### Safety Analysis

Safety is evaluated by the frequency of AEs, severity and types of AEs, and by changes from baseline in patients' vital signs, weight, and clinical laboratory results using the safety analysis set. Exposure to study drug and reasons for discontinuation are tabulated. TEAEs that occur after administration of the first dose of study and through 30 days after the last dose of study drug are tabulated.

AEs are tabulated according to the MedDRA and will include the following categories: (1) TEAEs; (2) Drug-related TEAEs; (3) Grade 3 or higher TEAEs; (4) Grade 3 or higher drug-related TEAEs; (5) The most commonly reported TEAEs (i.e., those reported by ≥10% of all patients); (6) SAEs (related and regardless of relationship); and (7) TEAEs leading to study drug modification and discontinuation.

### Results

### AB79-1002 Study—AB79 in Combination With (a) Lenalidomide And Dexamethasone (LenDex), And (b) Lenalidomide, Dexamethasone, And Bortezomib (VRd)

AB79-1002 is an open-label, multicenter Phase 1b Study investigating the safety and tolerability of AB79 in combination with backbone regimens ((a) lenalidomide and dexamethasone or (b) lenalidomide, dexamethasone, and bortezomib) for the treatment of patients with newly diagnosed multiple myeloma (NDMM) and for whom stem cell transplantation is not planned as initial therapy. Patients eligible for study enrollment have NDMM that has not been previously treated and for whom stem cell transplantation is not planned as first line therapy. The study was designed to determine the recommended phase 2 dose (RP2D) and to provide a preliminary assessment of the AB79 combinations against NDMM. Parameters such as safety, tolerability, pharmacokinetics (PK), pharmacodynamics (PD), and disease response were assessed. Ten (10) patients have been enrolled. In this ongoing study, clinical safety data includes that from patients who received at least 1 dose or where exposed to multiple doses in at least 1 if not multiple cycles at doses of 300 or 600 mg combined with standard doses of the backbone regimen. The most common TEAES (in ≥2 patients) regardless of causality in the total population, regardless of AB79 dose of combination partner, as of this date are decreased lymphocyte count (5 patients), diarrhea (3 patients), and abdominal pain, chills, dysgeusia, fatigue, muscle spasms, nausea, neutropenia, peripheral edema, and sinusitis (2 patients each). No DLTs were reported. There were no drug-related serious AEs, AEs that resulted in any drug discontinuation, or on-study deaths. The TEAEs in this combination study are consistent with the reported safety profile of the individual agents in the combination regimen and were generally expected on the basis of single agent AB79 (study AB79-1501) and clinical experience with the VRD and RD backbone regimens.

As of data cut-off, the preliminary objective response rate (ORR) for the total population, including both backbone regimens, was 100%, including deep (stringent CRs and VGPRs) and durable responses (at data cut-off exposure ranges from 1-11 cycles). Objective response determination is pending on one patient at this time.

### Example 3: A Phase 1/2A Open-Label, Dose-Escalation Study to Investigate the Safety and Tolerability, Efficacy, Pharmacokinetics, and Immunogenicity of AB79 Administered Subcutaneously as a Single Agent as Well as in Combination with Pomalidomide and Dexamethasone in Patients with Relapsed/Refractory Multiple Myeloma (RRMM) (AB79-1501)

This is a multicenter, dose-escalation, open-label, single-arm, Phase 1/2a study designed to determine the safety, tolerability, efficacy, PK, and immunogenicity of AB79 monotherapy in patients with RRMM, and to provide a preliminary assessment of its activity against MM. This study is an amendment to the Phase 1/2a study in RRMM described in Example 1 (a) to allow for an increase in the number of patients enrolled in the Phase 1 study; and (b) to add a cohort of patients to evaluate AB79 combined with the backbone regimen of pomalidomide and dexamethasone (PomDex) in patients with RRMM who have received at least 2 prior therapies and are refractory to the last therapy before study entry. PomDex is approved for this patient population and adding an anti-CD38 monoclonal antibody to it, especially one that is given subcutaneously (SC), could be beneficial and convenient for patients.

Thus, the primary objective of the Phase 1 portion of this study is to determine the safety and tolerability of (a) AB79 monotherapy and (b) AB79 combined with a backbone regimen of PomDex in patients with RRMM. The primary objective of the Phase 2a portion of this study is to provide a preliminary evaluation of the clinical activity of AB79 monotherapy in patients with RRMM.

The secondary objectives of the Phase 1 portion of the study are (a) to investigate a potential maximum tolerated dose/recommended phase 2 dose (MTD/RP2D) of AB79, as a single agent and when added to a backbone regimen of PomDex. The secondary objectives of the phase 2a portion of the study are (a) to further evaluate safety at the MTD/RP2D; (b) to provide a preliminary evaluation of time-to-event measures; (c) to further evaluate the immunogenicity of AB79; and (d) to further characterize the PK of AB79.

The exploratory objectives of this study are to explore potential biomarkers to test their correlation with clinical efficacy and safety parameters, including but not limited to (a) to characterize the pharmacodynamic profile of AB79 on immune cells (including CD38 occupancy); (b) to determine CD38 expression on MM cells and other immune cells before and during therapy; (c) to immunophenotype bone marrow aspirate (BMA) and whole blood cells including CD38+ immune cells at baseline and at different time intervals during treatment; and (d) to identify pharmacodynamic biomarkers including, but not limited to, B-cell receptor (BCR) and T-cell receptor (TCR) clonality, cytokines, chemokines, and complement proteins at baseline and at different time intervals during treatment.

The phase 1 portion of the study evaluates administration of single agent AB79 for dose-limiting toxicity (DLT) to determine the MTD or the RP2D for further assessment in phase 2a. A recommended dose below the MTD is identified based on the review of safety, PK, PD (e.g., CD38 occupancy), and clinical data from the phase 1 portion of the study. The safety and tolerability of AB79 is assessed by recording and analyzing TEAEs, dose modifications, treatment discontinuations, vital signs, physical examinations, serum chemistry and hematology, urinalysis, ECGs, and concomitant medications. In phase 1, approximately 6 doses of AB79 are evaluated in ascending cohorts of 3 to 6 patients per cohort. Cohorts may be expanded by enrolling additional patients to further inform selection of the RP2D before enrolling in the Phase 2a portion of the study. In the phase 2a portion of this study, Grade 4 or higher nonhematologic toxicity is monitored starting from the first 10 enrolled patients and then every 10 patients thereafter. Additionally, a cohort of patients receive AB79 combined with the backbone regimen of pomalidomide and dexamethasone (PomDex) in patients with RRMM who have received at least 2 prior therapies and are refractory to the last therapy before study entry.

Approximately 100 patients are enrolled in the study (approx. 55 patients in Phase 1 and 45 patients in Phase 2a). The maximum duration of treatment is expected to be 12 months for patients receiving monotherapy and approximately 18 months for patients in the combination cohort; however, patients with clinical benefit (per investigator and as agreed by the sponsor's study clinician) can continue on treatment with the explicit approval of the sponsor's study clinician.

AB79 injections in phase 1 are escalated as follows: 45 mg, 135 mg, 300 mg, 600 mg, 1200 mg, and 1800 mg. After patients have received premedication treatment, doses are administered with syringes as SC injections up to a maximum of 200 mg AB79 in 2 mL per injection. For dose levels where multiple SC injections are needed to administer the full prescribed dose (i.e., 300 mg dose and above), the Cycle 1 Day 1 dose is administered by giving each SC injection 30 minutes apart until the full scheduled dose has been administered. On all drug administration days after Cycle 1 Day 1, if the patient did not have an IR, the SC injections are given at the same time without a waiting period. For phase 1, each dose of AB79 is administered subcutaneously in each 28 day treatment cycle as once weekly for 8 weeks (8 doses), then once every 2 weeks for 16 weeks (8 doses), and then once every 4 weeks until PD or unacceptable toxicities occur. Patients receive ongoing treatment with AB79 until PD, unacceptable toxicities, or withdrawal due to other reasons. In the phase 1 combination cohort only, PomDex is administered per package instructions. See Table 3.

For phase 2a, in the absence of DLT, the dose is selected based upon review of the available safety, efficacy, PK, and PD information from the phase 1 portion of the study. Premedication is mandatory in phase 1 and phase 2a.

The study provides a Confirmation Cohort and a Combination Cohort. Each Cohort is administered a dose of 300 mg AB79 subcutaneously weekly for 8 weeks during Cycles 1 and 2 (8 doses), every other week during Cycles 3 to 6 for 16 weeks (8 doses), and then once every 4 weeks until PD in subsequent cycles. Safety and available efficacy, PK, and pharmacodynamics are reviewed at least after 6 patients in each subgroup have received 1 cycle of therapy, then on an ongoing basis. Further in this cohort, approximately 12 patients that have RRMM disease and are naïve to an anti-CD38 agent are enrolled. The Combination Cohort also receives pomalidomide and dexamethasone (PomDex), and given according to product labeling (Pomalyst USPI). Dexamethasone is administered IV or orally at 40 mg/day on Days 1, 8, 15, 22, or 20 mg/day given on Days 1, 8, 15, and 22 for patients over 75 years of age (Pomalyst USPI, Section 14.1). In the Combination Cohort, up to 6 patients are initially enrolled and safety in Cycle 1 reviewed. If 0 of 6 or 1 of 6 patients develop a DLT, an additional 12 patients are tested at the initial dose of AB79 [6 patients that are naïve to a prior anti-CD38 agent and 6 patients that have been exposed to a prior anti-CD38 agent], for an overall total of 18 patients. If 2 of 6 patients develop DLTs, an additional cohort of 6 patients is tested at a de-escalated dose; an intermediate or more conservative dose schedule may also be implemented as a means to provide an overall lower dose. A lower dose of pomalidomide is also considered based on available safety data. If 0 of 6 or 1 of 6 patients develop AB79-related DLTs at the revised dose, an additional cohort of up to 12 patients (with eligibility as above regarding anti-CD38 naïve or exposed) is tested at this dose level (in other words a dose lower than the initial dose, one that is intermediate or more conservative than the initial dose) or conservative dose schedule, for an overall total of 18 patients at the lower dose/conservative dose schedule. If 2 of 6 patients develop DLTs at the lower dose, this cohort is stopped for further evaluation.

Patients in Phase 1 dose Confirmation Cohort consists of adult patients with RRMM who have been previously treated with at least a PI, an IMiD, and a steroid. Patients have refractory disease or be intolerant to at least 1 PI and at least 1 IMiD, and they should have either received 3 or more prior therapies or received at least 2 prior therapies if one of those therapies included a combination of a PI and an IMiD. Patients who have had a previous autologous stem cell transplant will have additionally been exposed to an alkylating agent; however, patients who have not had a previous autologous stem cell transplant may not have been exposed to an alkylating agent per standard practice. Up to 6 patients are refractory to an anti-CD38 agent and approximately 12 patients in this cohort are anti-CD38 naïve.

Patients in Phase 1 Combination Cohort consist of adult patients with RRMM who have received at least 2 prior therapies including lenalidomide and a proteasome inhibitor and have demonstrated PD on or within 60 days of the completion of the last therapy. The first 6 patients enrolled are either naïve to a prior anti-CD38 antibody or may have been exposed to one previously. Once safety data have been reviewed, the following patients enrolled at the RP2D/MTD are: naïve to a prior anti-CD38 monoclonal antibody (approximately 6 patients) or exposed to a prior anti-CD38 monoclonal antibody (approximately 6 patients).

### Predose Medication: Phase 1 Dose Escalation and Dose Confirmation Cohorts

Patients receive the following premedications 1 to 3 hours prior to the start of AB79 administration on each dosing day: Dexamethasone: about 20 mg IV dose for the initial injection. Oral dexamethasone (about 20 mg) or an equivalent long-acting corticosteroid may be used before subsequent injections. Antipyretics: oral acetaminophen (650 to 1000 mg); Antihistamine: oral or IV diphenhydramine (25 to 50 mg, or equivalent); Montelukast 10 mg (or equivalent leukotriene inhibitor). Patients with a history of COPD may be prescribed postinfusion medications such as short- and long-acting bronchodilators and inhaled corticosteroids. After the first 4 infusions, if the patient experiences no major IRs, these additional inhaled postinfusion medication may be discontinued.

### Predose Medications: Phase 1 Combination Cohort (AB79-PomDex) Only

Patients will receive the following premedications 1 to 3 hours prior to the start of AB79 administration on each dosing day: Antipyretics: oral acetaminophen (650 to 1000 mg); Antihistamine: oral or IV diphenhydramine (25 to 50 mg, or equivalent); Montelukast 10 mg (or equivalent leukotriene inhibitor). Patients with a history of COPD may be prescribed postinfusion medications such as short- and long-acting bronchodilators and inhaled corticosteroids. After the first 4 infusions, if the patient experiences no major IRs, these additional inhaled postinfusion medications may be discontinued.

### Postdose Medications

Corticosteroid cream is applied topically to the injection site(s) and ice is applied locally for approximately 10 to 15 minutes. Patients may receive low-dose methylprednisolone (<20 mg) for the prevention of delayed injection-related reactions as clinically indicated after an injection.

### Main Criteria for Inclusion

Main Criteria for inclusion include the following. Male and female patients, aged ≥18 years, with ECOG performance status of ≤2, requiring additional therapy as determined by the investigator. Patients must have received the final dose of the following treatments/procedures within the specified minimum intervals before the first dose of AB79: 180 days for antibody therapy (including anti-CD38); 90 days for autologous transplantation; 14 days for chemotherapy, radiation therapy, and major surgery; and 7 days for corticosteroid therapy (up to systemic equivalent of 10 mg daily prednisone allowed). Patients must have adequate organ function as determined by the following laboratory values: absolute neutrophil count ≥1.0×10⁹/L; platelets ≥75,000/mm³ (≥75×10⁹/L); hemoglobin ≥7.5 g/dL; creatinine clearance ≥30 mL/minutes (Cockcroft-Gault formula); total bilirubin ≤1.5 times the upper limit of the normal range (ULN); and alanine aminotransferase/aspartate aminotransferase ≤2.5×ULN. Patients must have documented RRMM per the IMWG criteria, with measurable disease defined as one of the following: serum M-protein ≥0.5 g/dL (≥5 g/L) and urine M-protein ≥200 mg/24 hours. Patients without measurable M-protein in serum protein electrophoresis or urine protein electrophoresis must have a serum free light chain (FLC) assay result with involved FLC level ≥10 mg/dL (≥100 mg/L), provided serum FLC ratio is abnormal. Patients must have evidence of RRMM as defined by the IMWG criteria. For patients in the Escalation Cohort and Confirmation Cohort: previously received myeloma therapy including a proteasome inhibitor (PI), immunomodulatory drug (IMiD), and steroids; refractory or intolerant to at least 1 PI and at least 1 IMiD; either have received ≥3 prior lines of therapy or should have received at least 2 prior lines of therapy if one of those lines included a combination of PI and IMiD; in phase 1, previous exposure to an anti-CD38 agent, as a single agent or in combination, is allowed but is not required for patients in the Escalation Cohort. Patients in the Combination Cohort only: have undergone prior therapy with ≥2 prior anti-myeloma therapies; has either relapsed or relapsed and refractory disease; have progressed on or within 60 days of completing the last anti-myeloma therapy; patients may be either naïve or exposed to prior anti-CD38 monoclonal antibodies; however there is a cohort of patients refractory to at least 1 anti-CD38 mAb therapy at any time during treatment and one that is naïve to a prior anti-CD38 mAb. In the phase 2a portion of the study, up to 2 cohorts of patients with RRMM may be enrolled: one that is refractory to at least 1 anti-CD38 mAb therapy at any time during treatment and one that is naïve to prior anti-CD38 mAb.

### Primary and Secondary Endpoints:

In phase 2a, approximately 48 additional patients are treated to provide a preliminary estimate of the ORR in two expansion cohorts of patients with RRMM: up to 24 patients with RRMM that is anti-CD38 naive and up to 24 patients with RRMM that is refractory to an anti-CD38 therapy. Phase 2a of the study will also provide a more robust estimate of the safety profile at the MTD/RP2D.

No prospective calculations of statistical power have been made; however, the following table shows the width of the 80% CI, based on the observed ORR in a cohort size of 24 patients, for a range of observed response rates.

TABLE 11
A Summary of 80% CI Based on the Observed ORR

| Observed Rate | | | 42% | 50% | |
|---|---|---|---|---|---|
| of Response | 25% (6/24) | 33% (8/24) | (10/24) | (12/24) | 58% (14/24) |
| 80% CI (n = 24) | (13.7, 39.8) | (20.5, 48.4) | (27.7, 56.7) | (35.3, 64.7) | (43.3, 72.3) |

ORR: observed response rate. The observed rate of response is given as a percentage (n with response/N).

### Definitions of DLT

Toxicity is evaluated according to the NCI CTCAE, Version 4.03, effective Jun. 14, 2010. DLTs are defined as any of the following events regardless of relationship, except those events that are clearly due to extraneous causes: Grade 4 laboratory abnormalities, except those events that are clearly due to extraneous causes; Nonhematologic TEAEs of NCI CTCAE Grade A, except those events that are clearly due to extraneous causes, and occurring during the first cycle (except for Grade 3 nausea/vomiting that can be managed subsequently with antiemetics (Grade 3 nausea or vomiting that persists beyond 48 hours with or without appropriate medical intervention); Grade 3 fatigue lasting less than 3 days (approximately 72 hours); Grade 3 elevation of alanine aminotransferase or aspartate aminotransferase that resolves to Grade ≤1 or baseline within 7 days; Grade 3 IR that responds to symptomatic treatment (e.g., antihistamines, nonsteroidal anti-inflammatory drugs (NSAIDs), narcotics, IV fluids), without recurrence of Grade 3 symptoms); Hematologic TEAEs of NCI CTCAE Grade ≥4, except those events that are clearly due to extraneous causes, and occurring during the first cycle (except Grade ≥3 hemolysis, except those events that are clearly due to extraneous causes (e.g., negative direct Coombs test); Grade ≥3 low platelet count with clinically meaningful bleeding, defined as a blood loss of >100 cc or the requirement of a blood transfusion); An incomplete recovery from treatment-related toxicity causing a >2-week delay in the next scheduled injection before the initiation of Cycle 2.

For the purpose of dose escalation, DLTs are those events meeting the criteria above that occur before Cycle 2 Day 1 administration. TEAEs meeting DLT definitions occurring in later cycles determine the suitability of the MTD as the RP2D.

Patients who experience a DLT are withdrawn from study treatment unless the sponsor approves subsequent treatment in a lower dose cohort; such patients will not count as a patient in that lower dose cohort for escalation decisions.

In phase 1, patients who do not receive 4 full doses of AB79 within the 28-day (±2) treatment window or the Day 29 (i.e., Cycle 2, Day 1) assessment for reasons other than a DLT are replaced. Patients experiencing a DLT are not replaced.

A 3+3 dose escalation schema is used to inform dose escalation decisions and MTD/RP2D estimation. Initially, 3 patients are enrolled at the starting dose level. If none of the patients in a cohort of 3 patients exhibits a DLT during the 28-day cycle, then the dose may be escalated for the next cohort of 3 patients. If 1 patient in a cohort of 3 patients exhibits a DLT, then that cohort is expanded to a total of 6 patients. If ≤1 of 6 patients experiences a DLT, escalation will continue to the next higher dose level, at which 3 patients are enrolled. If 2 or more patients (2 or more out of 3, or 2 or more out of 6) experience a DLT, dosing will de-escalate to the next lower dose level, at which 3 additional patients are enrolled if 3 patients have been treated at that dose level. If 6 patients have been enrolled at the lower level with 1 or less DLT out of 6, dosing may stop and this dose level may be considered the MTD. The MTD is defined as the highest dose with a cohort of 6 patients having no more than 1 patient with a DLT.

Treatment for all cohorts uses a cycle length of 28 days. For a new cycle of treatment to begin, the patient must meet the following criteria: (a) absolute neutrophil count must be ≥1000/mm³; platelet count must be ≥75,000/mm³; (c) for therapy to resume, toxicity considered to be related to treatment with AB79 must have resolved to Grade or baseline, or to a level considered acceptable by the physician. If the patient fails to meet the above-cited criteria for retreatment, initiation of the next cycle of treatment is delayed for 1 week. At the end of that week, the patient is re-evaluated to determine whether the criteria for re-treatment have been met. If there is a delay of a subsequent cycle longer than 28 days because of a drug-related AE, the patient may be withdrawn from treatment unless there is clinical benefit as assessed by the investigator, with agreement by the sponsor's medical monitor; and (d) for AB79 injections within the same cycle, the decision of holding treatment is left to the investigator's discretion based on clinical and analytical data, and also based on the toxicity that the patient experienced with previous injections in the same cycle. The investigator should differentiate between acute toxicity (like an IR) from which the patient has recovered at the time of the next injection, and subacute toxicity (for example, neutropenia) that might be worsened upon another injection if it is not on a clear recovery path. If the dose cannot be administered on the scheduled day, the patient can be reviewed at the investigator's discretion in the following 48 hours. If AB79 cannot be administered within a cycle in this 48-hour window, the dose is missed and the patient scheduled for the next administration.

Patients experiencing AEs attributed to AB79 may continue study treatment with the same dose, may have AB79 treatment held or may be permanently discontinued from the study. Patients who have study drug held because of treatment-related or possibly related AEs may resume study drug treatment after resolution of the AE at the same dose level or at a reduced dose, depending on the nature and severity of the AE and whether it is the first occurrence or it is recurrent.

### Safety and Disease Assessments

In the phase 2a portion of this study, Grade 4 or higher nonhematological toxicity is monitored starting from the first 10 enrolled patients and then every 10 patients. If the stopping bounds of ≥4/10 and ≥6/20 are reached, accrual to the study is suspended to allow for investigation. After consideration by the study team, a decision is made as to whether accrual can be resumed. The bounds are based on a Bayesian strategy to monitor outcomes in clinical trials. If the stopping rule is met, there is 80% probability that the true toxicity rate is greater than 18% with a prior beta distribution with parameters 0.4 and 1.6 for the binomially distributed toxicity rate.

### Primary Endpoints

Primary endpoints in order of important for Phase 1 include the number of patients with TEAEs overall and per dose level; patients with DLTs at each dose level; patients with Grade ≥3 TEAEs; patients with SAEs; patients who discontinue because of TEAEs; and patients with dose modifications (delays, interruptions, dose reductions).

The primary endpoint for Phase 2a is ORR defined as the proportion of patients who achieved a partial response (PR) or better during study as defined by IMWG Uniform Response Criteria.

The primary endpoints for Phase 1 are RP2D based on both safety and efficacy outcomes as a single agent and when added to a backbone regimen of PomDex; summary statistics for the following PK parameters as a single agent and when added to a backbone regimen of PomDex (maximum observed concentration (Cₘₐₓ); time of first occurrence of Cₘₐₓ (tₘₐₓ); and area under the concentration-time curve from time 0 to the time of the last quantifiable concentration (AUCₗₐₛₜ)); Preliminary evaluation of antitumor activity of AB79, as single agent and in combination with PomDex, is assessed for patients with MM (by measuring ORR, defined as the proportion of patients who achieved a PR (50% tumor reduction) or better during the study as defined by the IMWG Uniform Response Criteria; proportion of patients who achieved minimal response (MR), defined as 25% tumor reduction, including in patients with disease measurable by serum FLCs; and anti-AB79 antibody incidence and characteristics.

The primary endpoints for Phase 2a are DLT-like frequencies and other TEAEs occurring over the course of extended treatment with AB79, including information about dose modification, treatment discontinuation, and vital signs; summary statistics for the following PK parameters: Cₘₐₓ, tₘₐₓ, and AUCₗₐₛₜ; Anti-AB79 antibody incidence and characteristics; proportion of patients who achieved MR, defined as 25% tumor reduction, is evaluated; duration of response (DOR), defined as the time from the date of the first documentation of response to the date of the first documented PD; PFS, defined as the time from the date of the first dose until the earliest date of PD, defined by IMWG criteria, or the date of death due to any cause; OS, defined as the time from the date of the first dose to the date of death due to any cause; time to response, defined as the time from the date of the first dose to the date of the first documentation of response (PR or better).

The exploratory endpoint of the phase 1 and 2a portions of the study is to explore potential biomarkers to test their correlation with clinical efficacy and safety parameters, including but not limited to: CD38 expression on MM cells and other immune cells before and during therapy; pharmacodynamic profile of AB79 on immune cells (including CD38 occupancy); immunophenotyping of BMA and/or whole blood cells including CD38+ immune cells at baseline and at different time intervals during treatment; and pharmacodynamic biomarkers including, but not limited to, BCR and TCR clonality, cytokines, chemokines, and complement proteins at baseline and at different time intervals during treatment.

### Primary Specimen Collection for PK, Pharmacodynamic, and Biomarker Assessments

Blood samples are collected via venipuncture or indwelling catheter at various time points for the measurement of serum concentrations of AB79 and for biomarker assessments (with the exception of BMA).

### PK Measurements

Serum samples for the measurement of concentrations of AB79 are collected at multiple time points. The timing, but not the total number, of samples may be modified during the study on the basis of emerging PK data if a change in the sampling scheme is considered necessary to better characterize the PK profile of AB79.

### Biomarkers and Pharmacodynamic Measurements

Several biomarkers are assessed to test for correlation with safety, PK, and, if possible, with efficacy. These biomarkers are used to identify patients who have a higher probability of response or of adverse reactions to AB79. Markers that are studied are markers linked either to the drug itself or to the treated disease. Markers of immune system activation are summarized using descriptive statistics.

### Pharmacodynamic and CD38 Occupancy Measurements

BMA samples are collected for analyzing CD38 expression on the surface of MM cells at screening, at the beginning of Cycles 2, 4, 7, and 13. CD38 occupancy assessment on MM cells in BMA is made during treatment. BMA samples that are collected at the beginning of Cycles 2, 4, 7, and 13 are also used for analyzing CD38 occupancy on MM cells. Occupancy assessment is performed centrally. Remaining cells from these samples are used for molecular characterization including, but not limited to, impact of Fc gamma receptor polymorphisms on efficacy and safety of AB79 and genotyping of the binding epitope of AB79.

The CD38 occupancy assessment measures the extent of CD38 occupancy by AB79 on the surface of CD38-expressing surrogate cells in circulation (e.g., PB, NK cells, and monocytes).

A blood sample for flow cytometry is drawn on Day 1 of every cycle and at the follow-up visit. These blood samples are analyzed for the presence and changes of immune cells by flow cytometry, such as B and T lymphocytes, monocytes, and NK cells. Blood samples are collected predose and at 24 hours after the first injection in Cycle 1 and 2, then predose at each following indicated visit. Remaining cells from these samples may be used for molecular characterization including, but not limited to, impact of Fc gamma receptor polymorphisms on efficacy and safety of AB79 and genotyping of the binding epitope of AB79.

### ADA Assessment

Serum samples for the assessment of anti-AB79 immunogenicity are collected at various time points. A blood sample is collected before administration of AB79 (i.e., prior to dosing on Day 1; baseline value), then subsequently before AB79 dosing at each designated visit (postbaseline values), and at visits for any patient who experiences a TEAE considered by the investigator to be consistent with hypersensitivity/IR. A sample is initially screened for ADA titer. If a sample is detected as ADA positive, it may be assessed for neutralizing activity.

### Direct and Indirect Coombs Testing

Serum samples for direct and indirect Coombs testing are collected at various time points.

### Completion of Study Treatment (for Individual Patients)

Patients receive AB79 until they experience PD, unacceptable toxicity, withdrawal of consent, death, or termination of the study by the sponsor. Patients have a follow-up visit 30 days after the last dose of study drug or prior to the start of subsequent alternative anticancer therapy, to permit the detection of any delayed AEs. Patients who discontinue study treatment for reasons other than PD continue PFS follow-up every 4 weeks from EOT until the occurrence of PD, death, the start of subsequent anticancer therapy, study termination, or until 12 months after discontinuation of study treatment, whichever occurs first. Patients are followed every 12 weeks for OS until death, loss to follow-up, consent withdrawal, or study termination. The duration of the study is approximately 42 months (3.5 years).

Discontinuation of Treatment with Study Drug and Patient Replacement

Study drug is permanently discontinued for patients meeting any of the following criteria: patient experiences an AE or other medical condition that indicates to the investigator that continued participation is not in the best interest of the patient; withdrawal by patient; and female patient has confirmed pregnancy. Treatment with study drug may also be discontinued for any of the following reasons: AE/SAE; protocol deviation; PD; symptomatic deterioration; unsatisfactory therapeutic response; study terminated by sponsor; lost to follow-up; or other.

Some patients may discontinue study drug for reasons other than PD before completing the full treatment course; these patients will remain in the study for posttreatment PFS assessments until PD occurs.

### Adverse Events

### Pretreatment Event Definition

A pretreatment event is any untoward medical occurrence in a patient or subject who has signed informed consent to participate in a study but before administration of any study medication; it does not necessarily have to have a causal relationship with study participation.

### Adverse Event (AE) Definition

AE means any untoward medical occurrence in a patient or subject administered a pharmaceutical product; the untoward medical occurrence does not necessarily have a causal relationship with this treatment. An AE can therefore be any unfavorable and unintended sign (including an abnormal laboratory finding), symptom, or disease temporally associated with the use of a medicinal (investigational) product whether or not it is related to the medicinal product. This includes any newly occurring event, or a previous condition that has increased in severity or frequency since the administration of study drug. An abnormal laboratory value will not be assessed as an AE unless that value leads to discontinuation or delay in treatment, dose modification, therapeutic intervention, or is considered by the investigator to be a clinically significant change from baseline.

### Serious Adverse Event (SAE) Definition

SAE means any untoward medical occurrence that at any dose: (a) results in death; (b) is life-threatening (refers to an AE in which the patient was at risk of death at the time of the event. It does not refer to an event which hypothetically might have caused death if it were more severe); (c) requires inpatient hospitalization or prolongation of an existing hospitalization; (d) results in persistent or significant disability (defined as a substantial disruption of a person's ability to conduct normal life functions) or incapacity; (e) is a congenital anomaly/birth defect; or (f) is a medically important event. This refers to an AE that may not result in death, be immediately life threatening, or require hospitalization, but may be considered serious when, based on appropriate medical judgment, may jeopardize the patient, require medical or surgical intervention to prevent one of the outcomes listed above, or involves suspected transmission via a medicinal product of an infectious agent. Examples of such medical events include allergic bronchospasm requiring intensive treatment in an emergency room or at home, blood dyscrasias or convulsions that do not result in inpatient hospitalization, or the development of drug dependency or drug abuse; any organism, virus, or infectious particle (eg, prion protein transmitting transmissible spongiform encephalopathy), pathogenic or nonpathogenic, is considered an infectious agent.

In this study, intensity for each AE, including any lab abnormality, is determined using the NCI CTCAE, Version 4.03, effective date 14 Jun. 2010. Clarification is made between an SAE and an AE that is considered severe in intensity (Grade 3 or 4), because the terms serious and severe are not synonymous. The general term severe is often used to describe the intensity (severity) of a specific event; the event itself, however, may be of relatively minor medical significance (such as a Grade 3 headache). This is not the same as serious, which is based on patient/event outcome or action criteria described above, and usually associated with events that pose a threat to a patient's life or ability to function. A severe AE (Grade 3 or 4) does not necessarily need to be considered serious. For example, a white blood cell count of 1000/mm³ to <2000 mm³ is considered Grade 3 (severe) but may not be considered serious. Seriousness (not intensity) serves as a guide for defining regulatory reporting obligations.

### Potential Risks

Based on the mechanism of action of AB79, potential AEs may include infusion or injection site reactions (IRs), cytokine release syndrome (CRS), hematological effects, and infections.

IRs are potentially dose-limiting AEs, not uncommonly associated with IV administration of biologic agents aimed at treating hematologic malignancies. IRs are less frequently associated with SC injection of these therapies. The ‘true’ clinical hypersensitivity reactions, antibody-mediated occur after repeat exposure. Symptoms of hypersensitivity range from mild skin rash to more severe reactions, wheezing, hypotension, poor perfusion, respiratory arrest, and rarely death. Non-anaphylactic clinical hypersensitivity occurs within the first hour; however delayed responses were reported. Symptoms of anaphylaxis, a potentially life-threatening condition, range from swelling, angioedema, bronchospasm, respiratory distress, and shock.

CRS represents an important IR often associated with the use of monoclonal antibodies used in anti-inflammatory and antitumor therapies. CRS may occur early in therapy, and often after the first infusion of the drug due to a high-level of activation of the immune system and engagement and proliferation of T-cells that can result in increased cytokine release. The CRS hallmark is fever. CRS also presents with rash, urticaria, headache, chills, fatigue, nausea, and/or vomiting. Severe cytokine release syndrome (SCRS) is characterized by severe dyspnea, often accompanied by bronchospasm and hypoxia, in addition to fever, chills, rigors, urticaria, and angioedema. This syndrome may be associated with some features of tumor lysis syndrome such as hyperuricemia, hyperkalemia, hypocalcemia, hyperphosphatemia, acute renal failure, and elevated lactate dehydrogenase, and may be associated with acute respiratory failure and death. The acute respiratory failure may be accompanied by events such as pulmonary interstitial infiltration or edema, visible on a chest x-ray. The syndrome frequently manifests within 1 or 2 hours of initiating the first infusion. Patients with a history of pulmonary insufficiency or those with pulmonary tumor infiltration may be at greater risk of poor outcome and is treated with increased caution. Patients who develop SCRS should have dosing interrupted immediately and should receive aggressive symptomatic treatment.

Hematologic effects may include reductions in platelets, lymphocytes, and RBCs.

Bacterial and/or viral infection secondary to immune suppression, such as for example, nasopharyngitis or upper respiratory infection may be observed.

### PK Analysis

PK parameters are estimated using noncompartmental analysis methods. Parameters are calculated for individual patients included in the PK analysis set using the AB79 concentration-time data. The calculated PK parameters will include, but not be limited to, Cₘₐₓ, tₘₐₓ, and AUCₗₐₛₜ (as permitted by the data). PK parameters are summarized using descriptive statistics. Individual AB79 concentration-time data and individual PK parameters are presented in listings and tabulated using summary statistics by dose cohort. Individual and mean concentration-time profiles are plotted by dose cohort. The PK data collected in this study may also contribute to future population PK analyses of AB79. These population PK analyses may include data collected in other AB79 clinical studies. The analysis plan for the population PK analysis is separately defined, and the results of these analyses are reported separately. Similarly, the time-matched PK and triplicate ECG data collected in this study may contribute to future concentration-QT interval corrected for heart rate (QTc) analyses. These analyses may include data collected in other AB79 clinical studies. The analysis plan for the concentration-QTc analysis is separately defined, and the results are reported separately.

### Biomarker Measurements

Serum samples are collected to monitor circulating biomarker changes (biomarkers including, but not limited to, cytokines, chemokines, and complement proteins) upon treatment. These biomarkers may be used to identify patients who have a higher probability of response or of experiencing adverse reactions to AB79. The biomarker sample analyses are performed if or when required. Because new techniques continue to be developed, the method and laboratory that are recommended for the biomarker analysis cannot be anticipated.

Serum samples for the assessment of circulating biomarkers are collected at the study visits. Blood samples are collected:
Cycle 1 Day 1: predose and approximately 24 hours after the first injection.
Cycle 1 Days 8, 15, and 22: predose.
Cycle 2 Day 1: predose and approximately 24 hours after the first injection.
Cycle 2 Days 8, 15, and 22: predose.
Cycle 3 Day 1 and Day 1 of each subsequent cycle: predose.
EOT visit.

### Immunogenicity Analyses

AB79 immunogenicity is analyzed using the immunogenicity analysis set by determining the proportion, incidence and characteristics (e.g., titer, transiently, and persistently ADA; and possible neutralizing activity) of patients with positive ADA (transient and persistent), and the proportion of patients in phase 2a with positive neutralizing ADA. NABs are also assessed in patients. Analysis is based on available data from patients with a baseline assessment and at least 1 postbaseline immunogenicity assessment. The impact of anti-AB79 antibodies on the PK profile, drug efficacy, and clinical safety are evaluated, if possible.

### Efficacy Analysis

The preliminary efficacy of AB79 for MM is evaluated by measuring the ORR (defined as the proportion of patients who achieved a PR or better during study) and the composition of sCR, CR, VGPR, and PR as defined by the IMWG Uniform Response Criteria. MR will also be analyzed. In addition, the efficacy of AB79 is assessed in patients by measuring DOR, PFS, and 1-year OS. TTR will also be measured.

### IMWG Criteria

IMWG Definition of MM: Clonal bone marrow plasma cells ≥10%* or biopsy-proven bony or extramedullary plasmacytoma* (clonality is established by showing κλ light-chain restriction on flow cytometry, immunohistochemistry, or immunofluorescence. Bone marrow plasma cell percentage should preferably be estimated from a core biopsy specimen; in case of a disparity between the aspirate and core biopsy, the highest value is used) and any one or more of the following myeloma-defining events: (a) evidence of end organ damage that can be attributed to the underlying plasma cell proliferative disorder, specifically; (b) hypercalcemia: serum calcium >0.25 mmol/L (>1 mg/dL) higher than the upper limit of the normal range or >2.75 mmol/L (>11 mg/dL); (c) renal insufficiency: creatinine clearance <40 mL per min or serum creatinine >177 μmol/L (>2 mg/dL); anemia: hemoglobin value of >20 g/L below the lower limit of normal, or a hemoglobin value <100 g/L; (d) bone lesions: one or more osteolytic lesions on skeletal radiography, CT, or PET-CT (if bone marrow has less than 10% clonal plasma cells, more than 1 bone lesion is required to distinguish from solitary plasmacytoma with minimal marrow involvement); (e) any one or more of the following biomarkers of malignancy: (i) clonal bone marrow plasma cell percentage ≥60%; (ii) involved: uninvolved serum free light chain ratio (FLC) ≥100; (iii) >1 focal lesions on MRI studies (these values based on the serum Freelite assay (The Binding Site Group, Birmingham, UK). The involved FLC must be ≥100 mg/L. Each focal lesion must be 5 mm or greater in size. See Table 12.

TABLE 12
IMWG Uniform Criteria for Response

| Category of | |
|---|---|
| Response | Response Criteria |
| sCR | Criteria for CR as defined below, with the addition of a normal FLC |
| | ratio, and an absence of clonal plasma cells by immunohistochemistry or |
| | 2- to 4-color flow cytometry; 2 consecutive assessments of laboratory |
| | parameters are needed (a). |
| CR | Negative immunofixation of serum and urine, disappearance of any soft |
| | tissue plasmacytomas, and <5% plasma cells in bone marrow; bone |
| | marrow; in patients for whom only measurable disease is by serum FLC |
| | level, normal FLC ratio of 0.26 to 1.65 in addition to CR criteria is |
| | required; 2 consecutive assessments are needed (b). |
| Immunophenotypic | sCR as defined, plus absence of phenotypically aberrant plasma cells |
| CR | (clonal) in bone marrow with minimum of 1 million total bone marrow |
| | cells analyzed by multiparametric flow cytometry (with >4 colors). |
| Molecular CR | CR as defined, plus negative allele-specific oligonucleotide polymerase |
| | chain reaction (sensitivity 10⁻⁵). |
| VGPR | Serum and urine M-protein detectable by immunofixation but not on |
| | electrophoresis, or ≥90% reduction in serum M-protein plus urine |
| | M-protein < 100 mg/24 hours; in patients for whom only measurable |
| | disease is by serum FLC level, >90% decrease in difference between |
| | involved and uninvolved FLC levels, in addition to VGPR criteria, is |
| | required; 2 consecutive assessments are needed (c). |
| PR | ≥50% reduction of serum M-protein and reduction in 24-hour urinary |
| | M-protein by ≥90% or to <200 mg/24 hours. |
| | If the serum and urine M-protein are not measurable, a ≥50% decrease in |
| | the difference between involved and uninvolved FLC levels is required |
| | in place of the M-protein criteria. |
| | If serum and urine M-protein are not measurable, and serum FLC is also |
| | not measurable, ≥50% reduction in bone marrow plasma cells is required |
| | in place of M-protein, provided the baseline percentage was ≥30%. |
| | In addition to the above criteria, if present at baseline, ≥50% reduction in |
| | the size of soft tissue plasmacytomas is also required. |
| | Two consecutive assessments are needed (a) no known evidence of |
| | progressive or new bone lesions if radiographic studies were performed. |
| Minimal response | ≥25% but ≤49% reduction of serum M-protein and reduction in 24-hour |
| (MR) (b) | urine M-protein by 50% to 89%. |
| | In addition to the above criteria, if present at baseline, 25% to 49% |
| | reduction in the size of soft tissue plasmacytomas is also required. |
| | No increase in size or number of lytic bone lesions (development of |
| | compression fracture does not exclude response). |
| | In this study, in patients that have myeloma measurable by serum free |
| | light chains, MR is defined as a reduction of ≥25 but ≤49% in the |
| | difference between involved and uninvolved FLC levels. |
| SD (c) | Does not meet the response criteria for CR (any variant), VGPR, PR, |
| | MR, or PD; no known evidence of progressive or new bone lesions if |
| | radiographic studies were performed. |

Source: Rajkumar, S. V. et al. (2011) Blood 117(18): 4691-5; Palumbo, A. et al. (2014) J. Clin. Oncol. 2014; 32(6): 587-600. CR: complete response; FLC: free light chain; IMWG: International Myeloma Working Group; MR: minimal response; ORR: overall response rate; PR: partial response; sCR: stringent complete response; SD: stable disease; VGPR: very good partial response.
(a) Clonality is established by showing κλ light-chain restriction on flow cytometry, immunohistochemistry, or immunofluorescence. Bone marrow plasma cell percentage should preferably be estimated from a core biopsy specimen; in case of a disparity between the aspirate and core biopsy, the highest value is used.
(b) For relapse-refractory myeloma only.
(c) These categories do not contribute to the ORR.

PD is defined as an increase of ≥25% from lowest response value in any of the following: (a) Serum M-protein (absolute increase must be ≥0.5 g/dL); serum M component increases ≥1 g/dL are sufficient to define relapse if starting M component is ≥5 g/dL), and/or (b) Urine M-protein (absolute increase must be ≥200 mg/24 hour), and/or (c) Only in patients without measurable serum and urine M-protein levels: the difference between involved and uninvolved FLC levels (absolute increase must be >10 mg/dL); (d) Only in patients without measurable serum and urine M-protein levels and without measurable disease by FLC levels, bone marrow plasma cell percentage (absolute percentage must be ≥10%). Alternatively, PD is defined as an increase of ≥25% from lowest response value in any of the following: (a) Definite development of new bone lesions or soft tissue plasmacytomas or definite increase in the size of existing bone lesions or soft tissue plasmacytomas, or (b) Development of hypercalcemia (corrected serum calcium >11.5 mg/dL) that can be attributed solely to the plasma cell proliferative disorder.

Clarifications to IMWG criteria for coding PD: Bone marrow criteria for PD are to be used only in patients without measurable disease by M-protein and by FLC levels; “25% increase” refers to M-protein, FLC, and bone marrow results, and does not refer to bone lesions, soft tissue plasmacytomas, or hypercalcemia, and the “lowest response value” does not need to be a confirmed value.

The ECOG scale for performance status is provided in Table 13.

TABLE 13
ECOG Scale for Performance Status

| Grade | Description |
|---|---|
| 0 | Normal activity. Fully active, able to carry on all |
| | predisease performance without restriction. |
| 1 | Symptoms but ambulatory. Restricted in physically |
| | strenuous activity, but ambulatory and able to carry |
| | out work of a light or sedentary nature (eg, light |
| | housework, office work). |
| 2 | In bed <50% of the time. Ambulatory and capable |
| | of all self-care, but unable to carry out any work |
| | activities. Up and about more than 50% of waking hours. |
| 3 | In bed >50% of the time. Capable of only limited self- |
| | care, confined to bed or chair more than 50% of |
| | waking hours. |
| 4 | 100% bedridden. Completely disabled. Cannot carry |
| | on any self-care. Totally confined to bed or chair. |
| 5 | Dead. |

### Clinical Chemistry, Hematology, and Urinalysis

Blood samples for analysis of the clinical chemistry and hematological parameters and urine sample parameters for analysis are shown in the tables below.

For Patients in Cohorts Receiving AB79 monotherapy, the following tests as shown in Tables 14 and 15 are performed.

TABLE 14
Chemistry and Hematology Tests for Research Purposes

| Hematology | Serum Chemistry | |
|---|---|---|
| ANC | Albumin | Creatinine clearance |
| Hematocrit | ALP | CRP |
| Hemoglobin | ALT | Glucose (nonfasting) |
| Platelet (count) | AST | GGT |
| Reticulocyte count | Bilirubin (total) | LDH |
| RBC count | BUN | Phosphate |
| WBC count with differential | Calcium | Potassium |
| Coagulation panel | Chloride | Sodium |
| | CO₂ (bicarbonate) | Total protein |
| | Creatinine | Urate (uric acid) |

ALP: alkaline phosphatase; ALT: alanine aminotransferase; ANC: absolute neutrophil count; AST: serine aminotransferase; BUN: blood urea nitrogen; CRP: C-reactive protein; GGT: γ-glutamyl transferase; LDH: lactate dehydrogenase; RBC: red blood cell; WBC: white blood cell.

TABLE 15
Clinical Urinalysis Tests for Research Purposes
Urinalysis

| | |
|---|---|
| Bilirubin | pH |
| Glucose | Protein |
| Ketones | Specific gravity |
| Leukocytes | Turbidity and color |
| Nitrite | Urobilinogen |
| Occult blood | |

For estimation of creatinine clearance, the Cockcroft-Gault formula is employed as follows: Estimated creatinine clearance=[(140−Age)·Mass (kg)]/[72·serum creatinine (mg/dL)]. For female patients, the result of the formula above is multiplied by 0.85.

For patients in the phase 1 Combination Cohort (AB79-PomDex) Cohort only, the following tests as shown in Tables 16, 17 and 18 are performed.

TABLE 16
Clinical Hematology and Chemistry: Standard
of Care Laboratory Tests

| Hematology | Chemistry | |
|---|---|---|
| Leukocytes with complete | Albumin | CO₂ |
| differential (total | | (bicarbonate) |
| neutrophils [ANC], | | |
| lymphocytes, monocytes, | | |
| eosinophils, and monocytes) | | |
| Platelet count | ALP | Creatinine |
| Hemoglobin | ALT | Estimated |
| | | glomerular |
| | | filtration rate |
| Serum pregnancy test | AST | Glucose |
| | B2-microglobulin | LDH |
| | Bilirubin (direct and | Potassium |
| | indirect) | |
| | Calcium | Sodium |
| | Chloride | Urate |

ALP: alkaline phosphatase; ALT: alanine aminotransferase; ANC: absolute neutrophil count; AST: serine aminotransferase; LDH: lactate dehydrogenase.

TABLE 17
Clinical Hematology and Chemistry: Tests for Research Purposes

| Clinical Hematology or | |
|---|---|
| Chemistry | Serology antibody titers |
| Coagulation panel (PT, PTT, INR) | Hepatitis B |
| Indirect and direct coombs | Hepatitis C |
| C-reactive protein | HIV |

INR: international normalized ratio; PT: prothrombin time; PTT: partial thromboplastin time.

TABLE 18
Clinical Urinalysis: Tests for Research Purposes
Urinalysis

| | |
|---|---|
| Bilirubin | pH |
| Glucose | Protein |
| Ketones | Specific gravity |
| Leukocytes | Turbidity, appearance, and color |
| Nitrite | Urobilinogen |
| Occult blood | Microscopic assessment (a) |

(a) Microscopic analyses are performed only as clinically indicated: bacteria, RBCs, WBCs, casts, and crystals.
After 24 months on treatment, the patient may be monitored according to standard clinical practice per the treating physician.

### Prestudy Prognostic Risk Assessment

A blood sample is collected for serum β₂ microglobulin at screening to assess patient disease status.

### Disease Response Assessments

Patients are assessed for disease response according to the IMWG criteria. In addition, in patients that have myeloma measurable by serum free light chains, MR is defined as a reduction of ≥25 but ≤49% in the difference between involved and uninvolved FLC levels.

For patients in the phase 1 Combination Cohort (AB79-PomDex), the following assessments as shown in Table 19 are performed.

TABLE 19
Myeloma Disease Assessments: Tests Standard of Care Tests

| Serum/Urine | Bone Marrow/Imaging |
|---|---|
| SPEP | Bone marrow biopsy and/or aspirate ^{(a)} |
| UPEP | Cytogenetics [presence of del (17), |
| | t(4:14), and t(14:16) at a minimum] |
| Immunofixation (serum | Imaging (skeletal survey, CT scan, |
| and urine) | PET/CT scan, MRI) |
| Quantification | |
| immunoglobulin levels | |
| Serum FLC | |

CT: computed tomography; MRI: magnetic resonance imaging; PET: positron emission tomography.
^{(a)} The BMAs only at Cycle 2D1, C4D1, C7D1, and C13D1 are for research purposes unless these align with a suspected CR then this procedure would be Standard of Care.

### Computed Tomography/Magnetic Resonance Imaging

Scans are performed at a minimum at screening and at the EOT visit. All treatment phase and follow-up scans should use the same imaging modality used at screening.

For patients with documented extramedullary disease, a whole-body x-ray, positron emission tomography-computed tomography (PET-CT) scan, computed tomography (CT) scan (includes low-dose CT), or magnetic resonance imaging (MM) scan are performed. The screening scan may be performed up to 21 days before first dose of AB79; however, if the patient has adequate image test performed within 5 weeks of the planned first dose of study drug, that image can be used as baseline and does not need to be repeated as part of screening. If disease is documented, then a repeat PET-CT scan, CT scan, or MM scan is performed as required to document response or PD.

Additional surveys (x-ray, CT, or MM) may also be performed at the investigator's discretion, e.g., in case of bone pain.

A blood sample for quantification of Ig (IgM, IgG, and IgA) is obtained at the screening visit, predose on Day 1 of every cycle, and at all visits.

A predose blood and 24-hour urine sample is obtained at the screening visit, Day 1 of every cycle, and at all visits.

Serum samples are obtained predose on Day 1 of every cycle and at all visits, for the serum FLC assay (including quantification of kappa and lambda chains and ratio).

Serum and urine samples are obtained for serum and urine immunofixation tests at the screening visit, predose on Day 1 of every cycle, to confirm CR, and at all response assessment visits.

BMAs are taken during the screening period and at the beginning of designated study visits at Cycles 2, 4, 7, and at Cycle 13. A BMA is obtained at screening for disease assessment (if a standard BMA was drawn within 5 weeks before consent, that BMA can be used as baseline and does not need to be repeated as part of screening unless cytogenetic evaluation is not available). A BMA will also be obtained at any time to assess CR or as needed to investigate suspected PD.

Patients who do not have historically documented cytogenetic results for the high-risk abnormalities of del (17), t(4:14), and t(14:16) will have cytogenetic evaluation performed on the BMA sample at screening. Cytogenetic evaluation may be performed using fluorescence in situ hybridization or conventional cytogenetics (karyotype). At a minimum, cytogenetic markers must include the 3 high-risk abnormalities of del(17), t(4:14), and t(14:16). Additional abnormalities (ampl 1q, del13, or del1p) may also be tested. Cytogenetics are analyzed locally, according to local standards.

### Results:

AB79-1501 Study—AB79 Alone or in Combination with Pomalidomide and Dexamethasone (PomDex)

As of the data cutoff, 5 patients have been treated with the AB79 plus PomDex combination. The most common TEAEs (in ≥2 patients) regardless of causality as of this date are neutropenia and cough (2 patients each). There have been no systemic reactions or injection site reactions have been rare. In the AB79 plus PomDex cohort, there has been 1 DLT (neutropenia) and evaluation of the MTD is ongoing. No drug-related SAEs or AEs leading to study drug discontinuation, or on-study deaths were reported with the combination.

AB79 showed early signs of antitumor activity as evidenced by at least 50% reduction in disease burden in some patients and minimal response defined as a 25% to 49% reduction in disease burden in others. As of data cut-off, the preliminary objective response rate (ORR) was 40% with a clinical benefit rate (defined as minor response or better) of 100%. Duration of response is not estimable.

### INCORPORATION BY REFERENCE

The contents of all cited references (including literature references, patents, patent applications, and websites) that may be cited throughout this application are hereby expressly incorporated by reference in their entirety for any purpose, as are the references cited therein, to the same extent as if each individual reference was specifically and individually indicated to be incorporated by reference in its entirety for any purposes.

### EQUIVALENTS

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein. Modifications for carrying out the invention that are obvious to persons of skill in the art are intended to be within the scope of the appended claims.

We claim:

1. A method of treating a subject having a CD38-positive hematological cancer, the method comprising administering to the subject a therapeutically effective amount of a) an anti-CD38 antibody or antigen binding fragment thereof, b) lenalidomide, and c) a corticosteroid for a time sufficient to treat the CD38-positive hematological cancer, wherein the anti-CD38 antibody comprises a variable heavy (VH) chain region comprising a CDR1 having the amino acid sequence of SEQ ID NO:3, a CDR2 having the amino acid sequence of SEQ ID NO:4, and a CDR3 having the amino acid sequence of SEQ ID NO:5; and a variable light (VL) chain region comprising a CDR1 having the amino acid sequence of SEQ ID NO:6, a CDR2 having the amino acid sequence of SEQ ID NO:7 and a CDR3 having the amino acid sequence of SEQ ID NO:8.

2. A method of treating a subject having a CD38-positive hematological cancer, the method comprising administering to the subject a therapeutically effective amount of a) an anti-CD38 antibody or antigen binding fragment thereof, b) pomalidomide, and c) a corticosteroid for a time sufficient to treat the CD38-positive hematological cancer, wherein the anti-CD38 antibody comprises a variable heavy (VH) chain region comprising a CDR1 having the amino acid sequence of SEQ ID NO:3, a CDR2 having the amino acid sequence of SEQ ID NO:4, and a CDR3 having the amino acid sequence of SEQ ID NO:5; and a variable light (VL) chain region comprising a CDR1 having the amino acid sequence of SEQ ID NO:6, a CDR2 having the amino acid sequence of SEQ ID NO:7 and a CDR3 having the amino acid sequence of SEQ ID NO:8.

3. The method of claim 1 or 2, wherein the VH chain region has the amino acid sequence of SEQ ID NO:9 and the VL chain region has the amino acid sequence of SEQ ID NO:10.

4. The method of claim 1 or 2, wherein the anti-CD38 antibody or antigen binding fragment thereof comprises a heavy chain amino acid sequence of SEQ ID NO:11 and a light chain amino acid sequence of SEQ ID NO:12.

5. The method of any one of claims 1-3, wherein the anti-CD38 antibody is an IgG1, IgG2, IgG3 or IgG4 isotype.

6. The method of claim 5, wherein the anti-CD38 antibody is the IgG1 isotype.

7. The method of claim 1 or 2, wherein the anti-CD38 antibody or antigen binding fragment thereof is fully human.

8. The method of claim 1 or 2, wherein the CD38-positive hematological cancer is multiple myeloma.

9. The method of claim 8, wherein the CD38-positive hematological cancer is newly diagnosed multiple myeloma (NDMM) or naïve multiple myeloma.

10. The method of claim 9, wherein the CD38-positive hematological cancer is newly diagnosed multiple myeloma (NDMM), and wherein the subject is a patient for whom stem cell transplantation is not planned as initial therapy.

11. The method of claim 1 or 2, wherein the CD38-positive hematological cancer has not been previously treated with a hematological cancer drug.

12. The method of claim 1 or 2, wherein the CD38-positive hematological cancer has not been previously treated with a multiple myeloma drug.

13. The method of claim 9, wherein the subject has refractory or relapsed multiple myeloma (RRMM).

14. The method of claim 1 or 2, wherein the anti-CD38 antibody or antigen binding fragment thereof is administered at a dose of about 300 mg once weekly for two treatment cycles, at a dose of about 300 mg once every two weeks for subsequent four treatment cycles and at a dose of about 300 mg once every four weeks for any treatment cycles thereafter, wherein a treatment cycle is 28 days.

15. The method of claim 1 or 2, wherein the anti-CD38 antibody or antigen binding fragment thereof is administered subcutaneously.

16. The method of claim 1 or 2, wherein the anti-CD38 antibody or antigen binding fragment thereof is administered in the absence of a hyaluronidase.

17. The method of claim 1, wherein the lenalidomide is administered at a dose of about 2.5 to about 25 mg daily for 21 days of each treatment cycle for up to 8 treatment cycles, wherein the treatment cycle is 28 days.

18. The method of claim 1 or 17, wherein the lenalidomide is administered orally.

19. The method of claim 2, wherein the pomalidomide is administered daily in a therapeutically effective amount for 21 days of each treatment cycle for up to 8 treatment cycles, wherein the treatment cycle is 28 days.

20. The method of claim 1 or 19, wherein the pomalidomide is administered orally.

21. The method of claim 1 or 2, wherein the corticosteroid is dexamethasone.

22. The method of claim 21, wherein dexamethasone is administered at a dose of about 20-40 mg weekly for 1-8 treatment cycles, wherein the treatment cycle is 28 days.

23. The method of claim 21, wherein dexamethasone is administered at a dose of about 40 mg weekly for 1-8 treatment cycles, wherein the treatment cycle is 28 days.

24. The method of claim any one of claims 21-23, wherein the dexamethasone is administered orally or intravenously.

25. The method of claim 1, further comprising administering a therapeutically effective amount of bortezomib.

26. The method of claim 25, wherein bortezomib is administered at a dose of about 0.7 to 1.3 mg/m² weekly for 3 weeks of 1-8 treatment cycles, wherein the treatment cycle is 28 days.

27. The method of claim 25 or 26, wherein the bortezomib is administered subcutaneously.

28. The method of claim 1, wherein a) the anti-CD38 antibody or antigen binding fragment thereof is administered on days 1, 8, 15 and 22 of the first two treatment cycles, on days 1 and 15 of the subsequent four treatment cycles and on day 1 of any additional treatment cycles; b) lenolidomide is administered on days 1 to 21 of each treatment cycle; and c) the corticosteroid is administered on days 1, 8, 15 and 22 of each of 1-8 treatment cycles, wherein the treatment cycle is 28 days.

29. The method of claim 2, wherein a) the anti-CD38 antibody or antigen binding fragment thereof is administered on days 1, 8, 15 and 22 of the first two treatment cycles, on days 1 and 15 of the subsequent four treatment cycles and on day 1 of any additional treatment cycles; b) pomolidomide is administered on days 1 to 21 of each treatment cycle; and c) the corticosteroid is administered on days 1, 8, 15 and 22 of each of 1-8 treatment cycles, wherein the treatment cycle is 28 days.

30. The method of claim 28, further comprising administering a therapeutically effective amount of bortezomib.

31. The method of claim 30, wherein bortezomib is administered at a dose of about 0.7 to 1.3 mg/m² weekly for 3 weeks of 1-8 treatment cycles, wherein the treatment cycle is 28 days.

32. The method of claim 31, wherein bortezomib is administered on days 1, 8, and 15 of each treatment cycle.

33. The method of claim 24, wherein dexamethasone is administered on days 1, 8, 15 and 22 of each treatment cycle.

34. The method of any one of the preceding claims, wherein the subject receives premedications 1 to 3 hours prior to the start of AB79 administration on each dosing day, and wherein the premedications comprise antipyretics and antihistamine.

35. The method of claim 34, wherein the antipyretics is acetaminophen, and is administered at a dose of about 650 to about 1000 mg orally.

36. The method of claim 34 or claim 35, wherein the antihistamine is diphenhydramine or equivalent, and is administered at a dose of about 25 mg to about 50 mg orally or intravenously.

37. The method of any one of claims 34-36, wherein the premedications further comprise montelukast or an equivalent leukotriene inhibitor.

38. The method of claim 37, wherein the montelukast or equivalent leukotriene inhibitor is administered at a dose of about 10 mg.


SEQUENCE LISTING (13 sequences)

SEQ ID NO: 1 - protein, 300 aa, Artificial Sequence
  Human CD38
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 301 aa, Artificial Sequence
  Cyno CD38
MANCEFSPVSGDKPCCRLSRRAQVCLGVCLLVLLILVVVVAVVLPRWRQQWSGSGTTSRFPETVLARCVKYTEVHPEMRHVDCQSVWDAFKGAFISKYPCNITEEDYQPLVKLGTQTVPCNKTLLWSRIKDLAHQFTQVQRDMFTLEDMLLGYLADDLTWCGEFNTFEINYQSCPDWRKDCSNNPVSVFWKTVSRRFAETACGVVHVMLNGSRSKIFDKNSTFGSVEVHNLQPEKVQALEAWVIHGGREDSRDLCQDPTIKELESIISKRNIRFFCKNIYRPDKFLQCVKNPEDSSCLSGI

SEQ ID NO: 3 - protein, 8 aa, Artificial Sequence
  HCDR1 AB79
GFTFDDYG

SEQ ID NO: 4 - protein, 8 aa, Artificial Sequence
  HCDR2 AB79
ISWNGGKT

SEQ ID NO: 5 - protein, 16 aa, Artificial Sequence
  HCDR3 AB79
ARGSLFHDSSGFYFGH

SEQ ID NO: 6 - protein, 8 aa, Artificial Sequence
  LCDR1 AB79
SSNIGDNY

SEQ ID NO: 7 - protein, 3 aa, Artificial Sequence
  LCDR2 AB79
RDS

SEQ ID NO: 8 - protein, 10 aa, Artificial Sequence
  LCDR3 AB79
QSYDSSLSGS

SEQ ID NO: 9 - protein, 135 aa, Artificial Sequence
  (VH) chain amino acid sequence
EVQLLESGGGLVQPGGSLRLSCAASGFTFDDYGMSWVRQAPGKGLEWVSDISWNGGKTHYVDSVKGQFTISRDNSKNTLYLQMNSLRAEDTAVYYCARGSLFHDSSGFYFGHWGQGTLVTVSSASTKGPSVFPLA

SEQ ID NO: 10 - protein, 129 aa, Artificial Sequence
  (VL) chain amino acid sequence
QSVLTQPPSASGTPGQRVTISCSGSSSNIGDNYVSWYQQLPGTAPKLLIYRDSQRPSGVPDRFSGSKSGTSASLAISGLRSEDEADYYCQSYDSSLSGSVFGGGTKLTVLGQPKANPTVTLFPPSSEEL

SEQ ID NO: 11 - protein, 453 aa, Artificial Sequence
  heavy chain (HC) amino acid
EVQLLESGGGLVQPGGSLRLSCAASGFTFDDYGMSWVRQAPGKGLEWVSDISWNGGKTHYVDSVKGQFTISRDNSKNTLYLQMNSLRAEDTAVYYCARGSLFHDSSGFYFGHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 216 aa, Artificial Sequence
  light chain (LC) amino acid
QSVLTQPPSASGTPGQRVTISCSGSSSNIGDNYVSWYQQLPGTAPKLLIYRDSQRPSGVPDRFSGSKSGTSASLAISGLRSEDEADYYCQSYDSSLSGSVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 13 - protein, 318 aa, Artificial Sequence
  Human CD157
MAAQGCAASRLLQLLLQLLLLLLLLAAGGARARWRGEGTSAHLRDIFLGRCAEYRALLSPEQRNKNCTAIWEAFKVALDKDPCSVLPSDYDLFINLSRHSIPRDKSLFWENSHLLVNSFADNTRRFMPLSDVLYGRVADFLSWCRQKNDSGLDYQSCPTSEDCENNPVDSFWKRASIQYSKDSSGVIHVMLNGSEPTGAYPIKGFFADYEIPNLQKEKITRIEIWVMHEIGGPNVESCGEGSMKVLEKRLKDMGFQYSCINDYRPVKLLQCVDHSTHPDCALKSAAAATQRKAPSLYTEQRAGLIIPLFLVLASRTQL